SEQUENCE LISTING (24 sequences)

SEQ ID NO: 1 - DNA, 7644 bp, Mus musculus
atggagccgcacgtgctgggcgccgggctctactggctgttgctgccctgcacgctcctggcggcctccctgttacgcttcaatgctctctcgctggtctatttgctgtttctactgctgctgccctggcttccaggcccctcaagacacagcataccaggtcacacaggtcgcctgctccgtgcactactctgcctcagcctcctcttcctggtggcccaccttgcctttcagatatgcctacacaccgtgcctcacctggaccagtttctgggacaaaacggtagcctttgggtgaaggtgtctcaacacataggggttacgaggctggacctgaaggacatctttaacaccaccaggctggtagcacctgacctgggagtgctgctggcgtcctccctttgccttggcctctgtggacgcctcacgaggaaagccgggcagagtcggcgcacccaggagctgcaggatgatgacgatgacgatgatgacgacgatgaagacatagatgctgccccagccgtggggctgaagggagcccctgccctggcaaccaaacgcaggctgtggctggcctcccgcttccgggtcacggcccactggctgctgatgacctctggacggacgctggtcattgtgttgctggccctggcaggcatagcccacccttcggccttctccagcatctacctggtggtgttcctggccatctgcacctggtggtcctgtcactttcctctcagccccctgggctttaacaccctctgtgtcatggtgagctgttttggtgccggccatctcatttgcctatactgctatcagacaccatttatccaggacatgttaccccctgggaacatctgggccaggctatttggtctcaagaacttcgtagacctccctaactactccagccccaacgccctggtactcaacactaagcacgcctggcccatctatgtgagtcccggaatcctgctgctgctatattacacagccacctctctcctgaagctccacaagagctgtccctcagagctgaggaaggagacacccagggaggatgaggagcatgagctggaactggaccacctggagccagagccccaggctagggacgccacccagggtgagatgcccatgaccacggaacctgaccttgacaactgcaccgtgcatgtactaaccagccagagccctgtccgccagcgtccagttcgccccaggctggctgagctgaaagagatgtcaccgctacatggcctgggccacctcatcatggaccagagctatgtgtgtgccctgattgccatgatggtgtggagcatcatgtaccacagttggctgaccttcgtcctgctgctctgggcctgcctcatctggacggtgcgtagccgtcaccagctggctatgctctgctcgccctgcatcctgctgtatgggctgacgctctgctgcctgaggtatgtgtgggccatggaacttcctgagctgcccaccaccctgggccctgtcagcctgcaccagttgggactggaacacacacgctacccttgcctggacctcggtgccatgctcctctatctgctcacattctggctccttctgcgtcagttcgtgaaggagaagctgctgaagaagcaaaaggtgcctgcggcgctgctggaggtcacggtggccgacactgagcccacacagacccagacgctgctgcggagcctgggggagctggtcaccggcatctacgtcaaatactggatctatgtgtgcgccggcatgttcattgtggtcagcttcgccggccgcctggtggtctacaaaatcgtctacatgttcctcttcctgctgtgcctcaccttgttccaggtctactacaccctgtggaggaagctgctacgtgtcttctggtggctcgtggtggcctatacaatgctcgtgctcatcgctgtgtacaccttccagttccaggacttccccacctattggcgcaacctcacgggcttcacagacgagcagttgggcgacctgggcctggagcagttcagtgtgtcggagctcttttccagtatcctcatccctggcttcttcctgctggcctgcatcctgcagctgcactacttccacagaccgttcatgcagctcactgacctggagcacgtgccgccaccaggcacccgccaccctcgatgggctcacaggcaggatgcagtgagcgaggcccctctgcttgagcatcaggaggaagaggaagtcttcagggaagatgggcagagcatggatgggccccaccaggccacacaggtccctgagggtacggccagcaagtggggcctggtggctgaccggctgctggacctagcggccagcttctcggctgtcctcacccgaatccaggtgttcgttcggcgcttgctagaacttcacgtcttcaagctggtggccctctacactgtctgggtggccctgaaagaagtgtctgtgatgaacctgctgctggtggtgctatgggccttcgccttgccctatccgcgcttccggcccatggcttcctgcctgtccaccgtgtggacctgtatcatcattgtatgcaagatgctctatcagctcaagattgtcaacccgcatgagtactccagcaactgcactgagcccttccccaacaataccaacttgcagcctttggagatcaaccagtctttgctgtaccgtggccctgttgaccctgccaactggtttggggtgcggaagggttaccccaacttgggctatatccagaaccacctgcagatccttctgttgctggtgtttgaggccgtggtgtaccggcgccaagagcactaccgccggcagcaccagcaggcccctctgcccgcccaggctgtgtgcgcagatggcacccgccagaggttggaccaggacctacttagctgcctcaagtatttcatcaacttcttcttctacaaattcgggctggagatctgcttcttgatggccgtgaatgtgattgggcagcgtatgaacttcatggtgatcttacacggttgctggttggtggccatccttacacgccggcgccgtgaggccatcgcccgcctctggcctaactactgtctgttcctcacgctgttcctgctgtaccagtacctgctgtgtttgggcatgccccctgcactgtgcattgactatccatggcgctggagcaaggccatccccatgaattccgccctcatcaagtggctgtacctacctgacttcttcagagcccccaactccaccaaccttatcagtgacttcctcctgctgctttgcgcctcccagcagtggcaggtcttctcagcggagcgaacggaggagtggcaacgcatggcgggcatcaacactgaccacctggagcccctgcgtggggagcccaaccctatacccaacttcatccactgcaggtcctatctggatatgctgaaggtggccgtcttccgctacctgttctggctggtgctcgttgtggtgtttgttgcgggggccacccgcataagcatcttcgggctggggtacctgctagcctgcttctacctgctgctgtttggcactaccctgctgcagaaggacacgcgagcccagctcgtgctgtgggactgcctcatcctctataatgtcactgtcatcatctctaagaatatgctgtcgctcctgtcctgtgtcttcgtggagcaaatgcagagcaacttctgctgggtcatccagctcttcagcctcgtgtgcacagtcaaaggctactatgatcccaaagagatgatgaccagggaccgggactgcctgctgcctgtggaggaggccgggatcatctgggacagtatctgcttcttcttcctgctcttgcaacggcgcatctttctcagccactacttcctgcatgtcagcgctgacctgaaagccacagccctgcaggcatccaggggctttgccctctacaatgcagccaacctgaagagcatcaacttccatcgccagattgaggagaagtccctggcccagctgaaaagacagatgaagcgcatccgtgccaaacaggagaagtacaggcagagccaggcaagtcgtggccaactccagtccaaagaccctcaggatcccagccaggagccagggcctgacagcccagggggctcctccccgccacggagacagtggtggcgcccctggctggaccacgccacagtcatccactctggcgactacttcctgtttgagtcagatagcgaggaggaagaggaggccctacctgaggaccccaggcctgcagctcagagtgccttccagatggcataccaggcatgggtaaccaatgcccagacagtgctgaggcagcgtcgggagcgggcacggcaggagcgggcagagcagctggcttctggaggtgacttgaacccagatgtggaaccagtagatgtcccagaagatgagatggcaggccgtagccacatgatgcagcgtgtgctaagcaccatgcagttcctgtgggtgctgggccaggccacggtagacgggctgacgcgctggctgcgtgcattcacgaagcaccaccgcaccatgagcgatgtgctgtgcgcagagcgctacctgctcacccaggagcttcttcgggttggagaggtacgccgaggtgtgctggaccagctttatgtgggtgaagatgaggccacattgtcaggtcccgtggagacccgggatggacccagcacagcctcaagtgggctgggagccgaagagcctttgagtagcatgacagacgacaccagcagccccctgagcacaggctataacacccgcagtggcagtgaggagattgtcaccgacgctggggacctccaggctgggacctccctgcacggctcccaagagcttttagccaatgctcgtacccggatgcgcacggccagcgagctgctactggataggcgcctgcatatccctgagctggaggaggccgagcggtttgaggcacagcagggccggactctgcggctgctcagggctgggtaccagtgcgtggcggcacactcggagctgctctgttacttcatcatcatccttaaccacatggtgacagcctcggctgcctccctggtgctgcccgtgcttgtgttcctgtgggccatgctgaccatcccgaggcctagcaagcgcttttggatgacagctatcgtcttcactgaggtcatggtggtcaccaaatacctgttccagttcggcttcttcccctggaacagctacgttgtgctgcggcgctatgagaacaagccctacttccctccgcgaatcctgggccttgagaaaacggacagctacatcaagtatgacctggtgcagctcatggccctcttcttccaccgctcgcagctactgtgttatggcctctgggaccatgaggaggatcgctatcccaaggaccattgcaggagtagtgtgaaggaccgggaggccaaggaagagccagaagctaagctggaatcgcagtctgagacaggcactgggcatcccaaggagccagtgttggccggtactcccagggaccacatccaagggaaaggaagtattagatccaaggatgttatccaagatcccccagaggaccttaagccccggcacacgaggcacatcagcatacgcttcaggaggcgcaaggagactccaggacccaaaggaacagcagtcatggagactgagcacgaggagggagaaggaaaagaaactacagagagaaagaggccgcgtcacactcaagaaaaatcgaagtttcgggagagaatgaaggcagctgggcgccggctgcagagcttctgtgtgtcactggcccagagcttctaccaacccttgcagcgtttcttccatgacattctgcacacaaagtaccgggcggccaccgacgtctacgccctcatgttcctggccgatattgtcgacatcatcatcatcatctttggtttttgggcttttgggaagcactctgcagccacagacattgcatcctcgctgtcagatgaccaggtgccacaggccttcctgttcatgctgctggtccagtttggcaccatggtcatcgaccgtgccctctacctgcgcaagactgtcctgggaaagctggcctttcaggtggtcctggtggtggcgattcacatctggatgttctttatcttaccggctgtcactgagaggatgttcagccagaatgcggtggcacagctgtggtacttcgtcaagtgcatttactttgccctgtccgcctaccagatccgctgtggctaccccacccgtatcttgggcaacttcctcaccaagaaatacaaccatctaaacctcttccttttccaggggttccgtctagtgccgttcctggtggagctgcgggccgtcatggactgggtgtggaccgacaccacgctgtccctgtccaactggatgtgtgtggaagacatctatgccaacatctttatcatcaagtgcagccgagagacagagaagaaatacccccagcccaaggggcagaagaagaagaaaattgtcaagtatggtatgggaggcctcattatcctcttcctcatcgccatcatctggttccctctgctcttcatgtcactgatccgctctgtggtcggggtcgtcaaccagcccattgatgtcaccgtcaccctcaagctaggcggctatgagccactgttcaccatgagcgcccagcagccatccattgtgccattcacaccccaggcctacgaggagctgtcccagcagtttgacccctatccactagccatgcagttcattagccagtacagtcctgaggacatcgtcactgcacagatcgagggcagctcgggggcgctgtggcgcatcagcccacccagccgagcccagatgaagcaggagctgtacaacggcacagccgacattacactgcgctttacctggaatttccaaagggacctggccaagggtggcactgtggagtatactaatgagaagcacaccttggagctggcccccaacagtacggcacgaaggcagctggcccaactgctcgagggcagacctgaccagtcagtggtcattccccatctcttccccaagtacattcgtgctcccaatgggcctgaagccaaccctgtgaagcagctgcagccagatgaggaagaggactaccttggtgtgcgcatccagctgcggagggagcaagtgggcacaggggcctctggggagcaagcgggcaccaaggcctccgacttcctcgagtggtgggtcatcgagctgcaggactgcaaggctgactgcaacctgctgcccatggtcatcttcagtgacaaggtcagcccacctagcctgggcttcctggccggctacgggattgtggggctgtacgtctccatcgtgctggtggttggcaagtttgtgcggggcttcttcagcgagatctctcactccatcatgttcgaggaactgccgtgtgtggaccgcatcctcaagctgtgccaggacatcttcttggtgcgcgagacccgggagctggagctggaggaggagctatacgccaagctcatcttcctgtaccgatctccagagaccatgattaagtggacacgtgagagggagtag

SEQ ID NO: 2 - protein, 2547 aa, Mus musculus
MEPHVLGAGLYWLLLPCTLLAASLLRFNALSLVYLLFLLLLPWLPGPSRHSIPGHTGRLLRALLCLSLLFLVAHLAFQICLHTVPHLDQFLGQNGSLWVKVSQHIGVTRLDLKDIFNTTRLVAPDLGVLLASSLCLGLCGRLTRKAGQSRRTQELQDDDDDDDDDDEDIDAAPAVGLKGAPALATKRRLWLASRFRVTAHWLLMTSGRTLVIVLLALAGIAHPSAFSSIYLVVFLAICTWWSCHFPLSPLGFNTLCVMVSCFGAGHLICLYCYQTPFIQDMLPPGNIWARLFGLKNFVDLPNYSSPNALVLNTKHAWPIYVSPGILLLLYYTATSLLKLHKSCPSELRKETPREDEEHELELDHLEPEPQARDATQGEMPMTTEPDLDNCTVHVLTSQSPVRQRPVRPRLAELKEMSPLHGLGHLIMDQSYVCALIAMMVWSIMYHSWLTFVLLLWACLIWTVRSRHQLAMLCSPCILLYGLTLCCLRYVWAMELPELPTTLGPVSLHQLGLEHTRYPCLDLGAMLLYLLTFWLLLRQFVKEKLLKKQKVPAALLEVTVADTEPTQTQTLLRSLGELVTGIYVKYWIYVCAGMFIVVSFAGRLVVYKIVYMFLFLLCLTLFQVYYTLWRKLLRVFWWLVVAYTMLVLIAVYTFQFQDFPTYWRNLTGFTDEQLGDLGLEQFSVSELFSSILIPGFFLLACILQLHYFHRPFMQLTDLEHVPPPGTRHPRWAHRQDAVSEAPLLEHQEEEEVFREDGQSMDGPHQATQVPEGTASKWGLVADRLLDLAASFSAVLTRIQVFVRRLLELHVFKLVALYTVWVALKEVSVMNLLLVVLWAFALPYPRFRPMASCLSTVWTCIIIVCKMLYQLKIVNPHEYSSNCTEPFPNNTNLQPLEINQSLLYRGPVDPANWFGVRKGYPNLGYIQNHLQILLLLVFEAVVYRRQEHYRRQHQQAPLPAQAVCADGTRQRLDQDLLSCLKYFINFFFYKFGLEICFLMAVNVIGQRMNFMVILHGCWLVAILTRRRREAIARLWPNYCLFLTLFLLYQYLLCLGMPPALCIDYPWRWSKAIPMNSALIKWLYLPDFFRAPNSTNLISDFLLLLCASQQWQVFSAERTEEWQRMAGINTDHLEPLRGEPNPIPNFIHCRSYLDMLKVAVFRYLFWLVLVVVFVAGATRISIFGLGYLLACFYLLLFGTTLLQKDTRAQLVLWDCLILYNVTVIISKNMLSLLSCVFVEQMQSNFCWVIQLFSLVCTVKGYYDPKEMMTRDRDCLLPVEEAGIIWDSICFFFLLLQRRIFLSHYFLHVSADLKATALQASRGFALYNAANLKSINFHRQIEEKSLAQLKRQMKRIRAKQEKYRQSQASRGQLQSKDPQDPSQEPGPDSPGGSSPPRRQWWRPWLDHATVIHSGDYFLFESDSEEEEEALPEDPRPAAQSAFQMAYQAWVTNAQTVLRQRRERARQERAEQLASGGDLNPDVEPVDVPEDEMAGRSHMMQRVLSTMQFLWVLGQATVDGLTRWLRAFTKHHRTMSDVLCAERYLLTQELLRVGEVRRGVLDQLYVGEDEATLSGPVETRDGPSTASSGLGAEEPLSSMTDDTSSPLSTGYNTRSGSEEIVTDAGDLQAGTSLHGSQELLANARTRMRTASELLLDRRLHIPELEEAERFEAQQGRTLRLLRAGYQCVAAHSELLCYFIIILNHMVTASAASLVLPVLVFLWAMLTIPRPSKRFWMTAIVFTEVMVVTKYLFQFGFFPWNSYVVLRRYENKPYFPPRILGLEKTDSYIKYDLVQLMALFFHRSQLLCYGLWDHEEDRYPKDHCRSSVKDREAKEEPEAKLESQSETGTGHPKEPVLAGTPRDHIQGKGSIRSKDVIQDPPEDLKPRHTRHISIRFRRRKETPGPKGTAVMETEHEEGEGKETTERKRPRHTQEKSKFRERMKAAGRRLQSFCVSLAQSFYQPLQRFFHDILHTKYRAATDVYALMFLADIVDIIIIIFGFWAFGKHSAATDIASSLSDDQVPQAFLFMLLVQFGTMVIDRALYLRKTVLGKLAFQVVLVVAIHIWMFFILPAVTERMFSQNAVAQLWYFVKCIYFALSAYQIRCGYPTRILGNFLTKKYNHLNLFLFQGFRLVPFLVELRAVMDWVWTDTTLSLSNWMCVEDIYANIFIIKCSRETEKKYPQPKGQKKKKIVKYGMGGLIILFLIAIIWFPLLFMSLIRSVVGVVNQPIDVTVTLKLGGYEPLFTMSAQQPSIVPFTPQAYEELSQQFDPYPLAMQFISQYSPEDIVTAQIEGSSGALWRISPPSRAQMKQELYNGTADITLRFTWNFQRDLAKGGTVEYTNEKHTLELAPNSTARRQLAQLLEGRPDQSVVIPHLFPKYIRAPNGPEANPVKQLQPDEEEDYLGVRIQLRREQVGTGASGEQAGTKASDFLEWWVIELQDCKADCNLLPMVIFSDKVSPPSLGFLAGYGIVGLYVSIVLVVGKFVRGFFSEISHSIMFEELPCVDRILKLCQDIFLVRETRELELEEELYAKLIFLYRSPETMIKWTRERE

SEQ ID NO: 3 - DNA, 8469 bp, Mus musculus
atggcttcggaagtggtgtgcgggctgatcttcaggctgctgcttcccatctgcctggcagtagcatgtgcgttccggtacaatgggctctcctttgtctaccttatctacctcttgctcattcctctgttctcagaaccaaccaaagcaacgatgcaaggacacacagggcggttgctacagtccctgtgcatcaccagcctctcatttctgctgcttcacatcattttccacatcacactggccagtctggaagctcaacaccgcatcacacctgcttacaactgctcaacatgggaaaagaccttccggcagattggctttgaaagcttgaagggagctgatgccggcaatggcatcagggtatttgtgcctgatatcgggatgttcattgcaagtctgaccatctggctggtctgcagaaccattgttaagaaaccagacacagaagaaatagcccaattgaattctgagtgtgaaaatgaagaattggctggaggagaaaagatggattcagaggaggcgctgatttatgaagaggacttagatggagaagaaggcatggaaggagagctagaagaaagcacaaaactaaaaatacttcgcaggttcgcctctgtcgcctccaaactgaaggagttcattggcaatatgatcaccacggcgggcaaggtcgtggtgaccatcctactgggctcctcgggcatgatgctgccatctttgacctcggctgtgtacttctttgtatttctgggcctgtgtacctggtggtcctggtgccgaactttcgacccattgctgttcggctgcctctgtgtcctgctggcaatcttcactgctgggcacctgattggactttatctgtaccagttccaattcttccaagaagcggtcccgcccaatgactactatgcaaggttgtttgggatcaagtccgttatccagacagactgtgccagcacctggaagatcatagtaaacccagacctgtcctggtatcaccatgccaaccccatcctcctgctggtgatgtactatacgctggccacgctgattcgcatctggctacaagagcctcttgtgcaagaggagatggcgaaggaagatgagggtgccctggattgcagctccaatcagaacacggcagagaggaggcggagcctgtggtatgcaacccagtacccaacagatgagagaaaacttttatccatgacccaagatgactacaaaccatctgatggcctgctggtgaccgtgaatggcaaccctgtggactaccacaccatccacccaagcctgcccatagagaatggccctgccaagacggacctgtacaccactccccagtaccggtgggagccttcggaggagtcctcagaaaagaaagaggaagaagaggacaagagagaagactccgaaggggaagggagccaggaggagaaaaggagtgtgagaatgcacgccatggtcgccgtgttccagttcatcatgaagcagagttacatctgtgccctcatcgccatgatggcatggagcatcacctaccacagctggctgactttcgtgctgttgatctggtcctgcaccctctggatgattcgcaacaggaggaagtatgccatgatcagttctcctttcatggtcgtctatgcgaatctgctgctggtgctgcagtacatatggagcttcgagctccccgagattaagaaggtccccgggtttctagaaaagaaagagccgggagaacttgcttcaaagattctcttcaccatcacgttctggctgctcctgaggcagcatctcacagagcagaaggccctgagggaaaaggaagcacttctttctgaagtcaaaattggaagtcaagaacttgaagaaaaggaagatgaggagctccaagatgtacaagtggaaggggagcccacagagaaggaagaagaagaggaagaggaaataaaggaagaaaggcatgaggtaaagaaggaagaggaagaggaagtggaggaagatgatgaccaggacatcatgaaggtgctgggtaatctggtggtggccctcttcatcaagtattggatctacgtctgcgggggcatgttcttttttgtcagctttgaggggaaaatcgtcatgtacaaaatcatctacatggtgctgttcctgttctgcgtggccttgtaccaggtgcactacgagtggtggaggaagatcctgaagtacttctggatgtcggtggtcatttataccatgttggtgctcatcttcatatacacttaccagtttgaaaacttcccaggcctgtggcaaaacatgaccggattaaagaaagaaaagctcgaggacctcggcctgaagcagttcacggtcgctgaactgttcactcgcatattcatccccacctcctttctactcgtctgcatcctacacctccactacttccacgatcggttcctggaacttacagacctcaagtccatccccagcaaagaagacaacaccatctacagccatgccaaagtcaatggtcgcgtgtacctaataataaataggttggcccaccctgaaggaagtcttccagacttagccatcatgaatatgacggccagccttgataagcctgaggtgcagaagttggcagagtctggggaggagaggccagaagagtgtgtcaagaagacggagaagggtgaggctgggaaagacagtgatgagtctgaggaggaggaagatgaagaagaggaatccgaggaagaagaaagctcagacctcaggaacaagtggcacctggtgattgaccggctcactgtgctctttctaaaattcctggagtatttccacaagttgcaggtgtttatgtggtggatcttggagctgcacatcatcaaaatcgtctcatcgtacattatctgggtgactgtgaaagaggtgtctctgttcaactatgtatttttgatttcttgggcatttgctctgccatatgccaaactccgtcgtgcggcttccagtgtctgcactgtctggacatgtgtgatcatcgtctgcaaaatgttgtaccagctgcaaaccattaaacctgagaacttttctgtcaactgctccttgcccaatgaaaatcaaaccaacattccccttcatgagttgaacaagtctcttctctacagtgcccctgttgatcccactgagtgggtaggcctgaggaagtcctctcctttgctcgtctacctgaggaacaacctcctgatgctggcgattttggcctttgaagtcaccgtttaccgccatcaggagtactatcgaggccgaaacaacctgactgctccagtgtctaaaactatctttcatgacatcacaaggttacatctagatgatgggcttatcaattgtgccaaatattttgtaaattacttcttctacaagtttggcctagagacctgtttcctaatgtccgtgaatgtaattggacagcgaatggacttctacgccatgattcatgcctgttggttgatcggtgtcttgtatcgacgcagaagaaaagccattgccgaggtctggcccaagtactgctgcttcctggcttgcatcatcaccttccagtacttcgtctgcatcggcatcccccctgccccttgccgagattatccatggagatttaagggagcctacttcaatgataacattatcaagtggctgtacttcccggatttcattgtgcggcccaaccccgtgtttcttgtctatgacttcatgctgctcctgtgtgcttccttacaacgacagatctttgaggacgaaaacaaggcagccgtgcgcatcatggccggggacaatgtcgagatctgcatgaaccttgatgcagcctcgttcagccagcataaccctgtgccagatttcattcactgcagatcttacttggacatgtccaaagtgatcatcttcagctacctcttctggttcgtcctcaccatcatcttcattacgggaacaaccaggatcagcatattttgcatgggctacttggtggcctgtttctatttcctgctctttggaggggatctgctgctgaagcccatcaagagcatccttcgttactgggactggctgattgcatacaacgtgtttgtgatcaccatgaagaatatactgtcaataggagcctgtggatacattggcgcactagtaagaaacagctgctggttgatccaagcgttcagcctggcctgcaccgtgaaaggctatcagatgcctgaagatgactccagatgcaagctgcccagtggggaggcagggatcatctgggacagcatctgctttgcgtttctgctgctgcagaggagagtcttcatgagctactacttcctgcatgtcgtggctgacattaaagcatcccagatcctggcatccagaggggccgaacttttccaggctacaattgtgaaagctgtaaaggcaagaattgaagaagagaaaaaatccatggaccagctgaagagacagatggatcgcatcaaagcccggcaacaaaagtacaaaaagggtaaggagaggatgctgagcttgacccaggagtcaggggaaggccaggacatccagaaagtctcagaagaagatgatgaaagagaagcagacaaacagaaagccaagggcaaaaaaaagcagtggtggcggccttgggttgatcatgcttccatggtcaggagtggagattattatttgtttgaaacggatagtgaagaggaggaagaagaggaacttaaaaaggaggatgaagaacccccacggaagtcagcattccagttcgtctaccaagcctggattactgatcctaaaacggcactccgacagaggaggaaagagaagaaaaagttggccagagaagagcagaaggagagacgcaaaggatctggggacggtccagtggaatgggaagatcgagaggatgagccagtcaaaaagaaatctgatggaccagataatatcatcaaraggatatttaatatcctgaagttcacatgggttctgttcctggccacagtggatagtttcaccacttggctcaactccatctcaagggaacacatcgacatatccacagtcctgcggattgaaagatgcatgctgactcgagagataaaaaagggcaatgttccaacacgggaaagcatccacatgtactaccagaaccacattatgaacctttcccgagaatccggcctggacacaatcgacgagcactcgggggctggctccagggcccaggcagcacataggatggatagcttagattcccgggacagtatctccagctgctacactgaagcgaccctgctgatctcaaggcagtccacccttgatgatttagatggacaagaccccgttcctaaaacaagcgagcgtgctcggcctaggctccgtaaaatgttcagcctggacatgtcctcttcgtcagctgacagtggcagtgtagcatcaagcgagcccacacagtgtaccatgctgtattcgcgccaaggaactacggaaaccatcgaggaggtggaggctgaagctgaggaggaggtggtagaaggtctggagcctgagctccatgatgcagaagaaaaggaatacgcagctgaatatgaagcaggggtagaggagatcagcctcactccagatgaggaattgcctcagttctctacagatgactgtgaggcgccgccctcttacagcaaggctgtcagctttgagcatctatcgtttgcgtcgcaagatgactctggggccaagaaccacatggtggtcagtcctgatgacagccgtacagacaagctggagtcaagcatcttaccacctctgactcatgagctgacagccagcgatctgctgatgagcaagatgttccatgatgatgagcttgaggagtctgagaagttctacgtggaccagccccggtttttgctgctattctatgccatgtataacaccctggtggcccgctcagaaatggtgtgctatttcgtgattatcctgaaccacatgacctctgcctccatcatcactctcctgttgcccatcttgatcttcctctgggccatgctgtctgttcccaggcccagccgacgcttctggatgatggccatcgtctacactgaggtggcgatcgtagtgaaatacttcttccagttcggcttctttccctggaataaggacttggaaatatacaaggaaaggccctactttccaccgaacatcatcggggtggagaagaaggagggctatgttctttatgacctcattcagctgctggctctattcttccatcgctccattctgaagtgtcatggtctatgggacgaggatgacattgttgacagtaacacagacaaggaaggatcggatgacgagctgtccctcgatcagggcagaagaggctcctcagactcactgaagtctatcaacctggctgcctctgtagagtctgtccacgtgaccttccccgagcagccagcggccatcaggagaaaacgctcctgcagtagctctcagatctccccacggtccagcttctcttcaaataggtccaaaagaggcagcacaagcacccggaacagtagccagaaaggaagcagtgttttaagccttaaacagaaaagcaaacgagaactttacatggaaaagcttcaagagcatttaatcaaagcaaaggcgtttaccataaagaagactctacagatctatgtccccattcggcaattcttctatgacctcatccacccagactacagtgctgtgactgatgtatacgtgctgatgtttctggctgacactgtagacttcattatcatcgtctttggattctgggcctttgggaaacactcggcagctgcagatatcacctcttctctatcagaagaccaggtccccgggccattcctggtgatggtcctcattcagtttggaaccatggtggtggaccgtgcactctacctcaggaagactgtgctgggcaaggtcatcttccaggtcatcctcgtgtttgggattcacttctggatgttcttcatcttacccggtgtgactgagaggaaattcagccagaacctggtggcacagctttggtactttgtgaagtgtgtgtactttgggctgtctgcctatcaaatccgctgtggctacccaacccgtgtgctcgggaacttcctcacaaagagctacaattacgtcaacctcttcttgtttcaagggttccgcctggtcccatttctgactgagctgcgggctgtgatggattgggtgtggacagatacaaccctgagcctctccagctggatttgtgtggaggacatttacgcgcacatttttatcttaaagtgctggcgagagtcggaaaagagataccctcaacccaggggtcagaagaagaagaaagccgtgaagtacggcatgggcggcatgatcatcgtccttctcatctgtatcgtctggtttcctctgctcttcatgtctttgatcaaatctgtcgcaggggttatcaaccaacccctggatgtgtccgtcacaattaccctgggaggttatcagcctatcttcacaatgagcgcccagcaaagccagctgaaagttatggataattcaaagtataatgaatttctaaaatctttcggccccaacagtggcgcaatgcaatttctggagaactatgaaagggaagacgtaacagtagcagaactggaagggaactcgaattctttgtggaccatcagccctcccagtaagcagaaaatgatacaggagctcaccgaccccaatagttgcttctctgttgttttttcatggagtatccagagaaacatgactttgggtgcaaaagccgaaatagcaacggataagctttctttccctcttgcggtcgcaacacggaatagcatagctaaaatgattgctggcaatgacacagaaagttcaaacacgccagtgacaatagagaagatctacccatactatgtgaaggcacccagcgattcaaactcaaagcctataaaacagcttctgtctgaaaataatttcatgaatatcaccatcattttgttcagagacaatgtcacaaagtccaatagtgagtggtgggttctcaacctgaccggaagtaggatattcaaccaggggtcccaagccttggaactggtggtcttcaatgacaaagtcagccctccaagtctaggcttcttggctggctatggtatcatgggattgtatgcatctgttgtccttgtaattgggaaatttgttcgtgagttcttcagtgggatctctcattccatcatgtttgaagagcttccaaatgtggacagaatcttgaagttgtgcacagatatattcctcgtgagggagacaggggagttggaactggaggaagacctctacgccaagttgatattcctgtacaggtcaccagaaaccatgatcaagtggactagagaaaaaacaaactga

SEQ ID NO: 4 - protein, 2822 aa, Mus musculus [sequence omitted]

SEQ ID NO: 5 - DNA, 21 bp, Artificial Sequence
  Mouse Piezo1 siRNA
caccggcatctacgtcaaata

SEQ ID NO: 6 - DNA, 21 bp, Artificial Sequence
  Mouse Piezo1 siRNA
accaagaaatacaaccatcta

SEQ ID NO: 7 - DNA, 21 bp, Artificial Sequence
  Mouse Piezo1 siRNA
tcggcgcttgctagaacttca

SEQ ID NO: 8 - DNA, 21 bp, Artificial Sequence
  Mouse Piezo1 siRNA
cggaatcctgctgctgctata

SEQ ID NO: 9 - DNA, 19 bp, Artificial Sequence
  Mouse Piezo1 siRNA
gaaagagatgtcaccgcta

SEQ ID NO: 10 - DNA, 19 bp, Artificial Sequence
  Mouse Piezo1 siRNA
gcatcaacttccatcgcca

SEQ ID NO: 11 - DNA, 19 bp, Artificial Sequence
  Mouse Piezo1 siRNA
aaagacagatgaagcgcat

SEQ ID NO: 12 - DNA, 19 bp, Artificial Sequence
  Mouse Piezo1 siRNA
ggcaggatgcagtgagcga

SEQ ID NO: 13 - DNA, 19 bp, Artificial Sequence
  Mouse Piezo2 siRNA
gaatgtaattggacagcga

SEQ ID NO: 14 - DNA, 19 bp, Artificial Sequence
  Mouse Piezo2 siRNA
tcatgaaggtgctgggtaa

SEQ ID NO: 15 - DNA, 19 bp, Artificial Sequence
  Mouse Piezo2 si RNA
gattatccatggagattta

SEQ ID NO: 16 - DNA, 19 bp, Artificial Sequence
  Mouse Piezo2 siRNA
gaagaaaggcatgaggtaa

SEQ ID NO: 17 - DNA, 7566 bp, Homo sapiens
atggagccgcacgtgctcggcgcggtcctgtactggctgctgctgccctgcgcgctgctggctgcctgcctgctccgcttcagcggactctcgctggtctacctgctcttcctgctgctgctgccctggttccccggccccacccgatgcggcctccaaggtcacacaggccgcctcctgcgggcattgctgggcctcagcctgctcttcctggtggcccatctcgccctccagatctgcctgcatattgtgccccgcctggaccagctcctgggacccagctgcagccgctgggagaccctctcgcgacacataggggtcacaaggctggacctgaaggacatccccaacgccatccggctggtggcccctgacctgggcatcttggtggtctcctctgtctgcctcggcatctgcgggcgccttgcaaggaacacccggcagagcccacatccacgggagctggatgatgatgagagggatgtggatgccagcccgacggcagggctgcaggaagcagcaacgctggcccctacacggaggtcacggctggccgctcgtttccgagtcacggcccactggctgctggtggcggctgggcgggtcctggccgtaacactgcttgcactggcaggcatcgcccacccctcggccctctccagtgtctacctgctgctcttcctggccctctgcacctggtgggcctgccactttcccatcagcactcggggcttcagcagactctgcgtcgcggtggggtgcttcggcgccggccatctcatctgcctctactgctaccagatgcccttggcacaggctctgctcccgcctgccggcatctgggctagggtgctgggtctcaaggacttcgtgggtcccaccaactgctccagcccccacgcgctggtcctcaacaccggcctggactggcctgtgtatgccagccccggcgtcctcctgctgctgtgctacgccacggcctctctgcgcaagctccgcgcgtaccgcccctccggccagaggaaggaggcggcaaaggggtatgaggctcgggagctggagctagcagagctggaccagtggccccaggaacgggagtctgaccagcacgtggtgcccacagcacccgacaccgaggctgataactgcatcgtgcacgagctgaccggccagagctccgtcctgcggcggcctgtgcggcccaagcgggctgagcccagggaggcgtctccgctccacagcctgggccacctcatcatggaccagagctatgtgtgcgcgctcattgccatgatggtatggagcatcacctaccacagctggctgaccttcgtactgctgctctgggcctgcctcatctggacggtgcgcagccgccaccaactggccatgctgtgctcgccctgcatcctgctgtatgggatgacgctgtgctgcctacgctacgtgtgggccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcccacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtgtccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggaggactccagggacgaggggctgggcgtggccactccccaccaggccacgcaggtgcctgaaggggcagccaagtggggcctggtggctgagcggctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcactcatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgactttcaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgccacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctcgctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagccagggtcggacccacggacgggaccccagaaccccaagtggagctcaggccccgtgatacgaggcgcatcagtctacgttttagaagaaggaagaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccactgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccctaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtag

SEQ ID NO: 18 - protein, 2521 aa, Homo sapiens
MEPHVLGAVLYWLLLPCALLAACLLRFSGLSLVYLLFLLLLPWFPGPTRCGLQGHTGRLLRALLGLSLLFLVAHLALQICLHIVPRLDQLLGPSCSRWETLSRHIGVTRLDLKDIPNAIRLVAPDLGILVVSSVCLGICGRLARNTRQSPHPRELDDDERDVDASPTAGLQEAATLAPTRRSRLAARFRVTAHWLLVAAGRVLAVTLLALAGIAHPSALSSVYLLLFLALCTWWACHFPISTRGFSRLCVAVGCFGAGHLICLYCYQMPLAQALLPPAGIWARVLGLKDFVGPTNCSSPHALVLNTGLDWPVYASPGVLLLLCYATASLRKLRAYRPSGQRKEAAKGYEARELELAELDQWPQERESDQHVVPTAPDTEADNCIVHELTGQSSVLRRPVRPKRAEPREASPLHSLGHLIMDQSYVCALIAMMVWSITYHSWLTFVLLLWACLIWTVRSRHQLAMLCSPCILLYGMTLCCLRYVWAMDLRPELPTTLGPVSLRQLGLEHTRYPCLDLGAMLLYTLTFWLLLRQFVKEKLLKWAESPAALTEVTVADTEPTRTQTLLQSLGELVKGVYAKYWIYVCAGMFIVVSFAGRLVVYKIVYMFLFLLCLTLFQVYYSLWRKLLKAFWWLVVAYTMLVLIAVYTFQFQDFPAYWRNLTGFTDEQLGDLGLEQFSVSELFSSILVPGFFLLACILQLHYFHRPFMQLTDMEHVSLPGTRLPRWAHRQDAVSGTPLLREEQQEHQQQQQEEEEEEEDSRDEGLGVATPHQATQVPEGAAKWGLVAERLLELAAGFSDVLSRVQVFLRRLLELHVFKLVALYTVWVALKEVSVMNLLLVVLWAFALPYPRFRPMASCLSTVWTCVIIVCKMLYQLKVVNPQEYSSNCTEPFPNSTNLLPTEISQSLLYRGPVDPANWFGVRKGFPNLGYIQNHLQVLLLLVFEAIVYRRQEHYRRQHQLAPLPAQAVFASGTRQQLDQDLLGCLKYFINFFFYKFGLEICFLMAVNVIGQRMNFLVTLHGCWLVAILTRRHRQAIARLWPNYCLFLALFLLYQYLLCLGMPPALCIDYPWRWSRAVPMNSALIKWLYLPDFFRAPNSTNLISDFLLLLCASQQWQVFSAERTEEWQRMAGVNTDRLEPLRGEPNPVPNFIHCRSYLDMLKVAVFRYLFWLVLVVVFVTGATRISIFGLGYLLACFYLLLFGTALLQRDTRARLVLWDCLILYNVTVIISKNMLSLLACVFVEQMQTGFCWVIQLFSLVCTVKGYYDPKEMMDRDQDCLLPVEEAGIIWDSVCFFFLLLQRRVFLSHYYLHVRADLQATALLASRGFALYNAANLKSIDFHRRIEEKSLAQLKRQMERIRAKQEKHRQGRVDRSRPQDTLGPKDPGLEPGPDSPGGSSPPRRQWWRPWLDHATVIHSGDYFLFESDSEEEEEAVPEDPRPSAQSAFQLAYQAWVTNAQAVLRRRQQEQEQARQEQAGQLPTGGGPSQEVEPAEGPEEAAAGRSHVVQRVLSTAQFLWMLGQALVDELTRWLQEFTRHHGTMSDVLRAERYLLTQELLQGGEVHRGVLDQLYTSQAEATLPGPTEAPNAPSTVSSGLGAEEPLSSMTDDMGSPLSTGYHTRSGSEEAVTDPGEREAGASLYQGLMRTASELLLDRRLRIPELEEAELFAEGQGRALRLLRAVYQCVAAHSELLCYFIIILNHMVTASAGSLVLPVLVFLWAMLSIPRPSKRFWMTAIVFTEIAVVVKYLFQFGFFPWNSHVVLRRYENKPYFPPRILGLEKTDGYIKYDLVQLMALFFHRSQLLCYGLWDHEEDSPSKEHDKSGEEEQGAEEGPGVPAATTEDHIQVEARVGPTDGTPEPQVELRPRDTRRISLRFRRRKKEGPARKGAAAIEAEDREEEEGEEEKEAPTGREKRPSRSGGRVRAAGRRLQGFCLSLAQGTYRPLRRFFHDILHTKYRAATDVYALMFLADVVDFIIIIFGFWAFGKHSAATDITSSLSDDQVPEAFLVMLLIQFSTMVVDRALYLRKTVLGKLAFQVALVLAIHLWMFFILPAVTERMFNQNVVAQLWYFVKCIYFALSAYQIRCGYPTRILGNFLTKKYNHLNLFLFQGFRLVPFLVELRAVMDWVWTDTTLSLSSWMCVEDIYANIFIIKCSRETEKKYPQPKGQKKKKIVKYGMGGLIILFLIAIIWFPLLFMSLVRSVVGVVNQPIDVTVTLKLGGYEPLFTMSAQQPSIIPFTAQAYEELSRQFDPQPLAMQFISQYSPEDIVTAQIEGSSGALWRISPPSRAQMKRELYNGTADITLRFTWNFQRDLAKGGTVEYANEKHMLALAPNSTARRQLASLLEGTSDQSVVIPNLFPKYIRAPNGPEANPVKQLQPNEEADYLGVRIQLRREQGAGATGFLEWWVIELQECRTDCNLLPMVIFSDKVSPPSLGFLAGYGIMGLYVSIVLVIGKFVRGFFSEISHSIMFEELPCVDRILKLCQDIFLVRETRELELEEELYAKLIFLYRSPETMIKWTREKE

SEQ ID NO: 19 - DNA, 8259 bp, Homo sapiens
atggcctcagaagtggtgtgcgggctcatcttcaggctgctgctgcccatctgcctggcagtagcatgtgcattccgatacaatgggctctcctttgtctaccttatctacctcttgctcattcctctgttctcagaaccaacaaaaacgacgatgcaaggacatacgggacggttattaaagtctctgtgcttcatcagtctttccttcctgttgctgcacatcattttccacatcacgttggtgagccttgaagctcaacatcgtattgcacctggctacaactgctcaacatgggaaaagacattccggcagatcggctttgaaagcttaaagggagctgatgctggcaatgggatcagagtgtttgtacctgacatcgggatgttcattgctagtctgaccatctggctcctctgtagaaacattgttcagaaacctgtgacagacgaagcagcacagagtaacccggagtttgaaaatgaagaattggctgaaggagaaaaaattgattcagaagaggcactgatctatgaagaggatttcaatggaggagatggtgttgaaggcgagttggaagaaagcacgaagttaaaaatgttccgcaggcttgcctctgtggcctctaagctcaaggagttcattggcaacatgatcaccactgctgggaaagtcgttgttaccatcttactgggctcctcgggcatgatgttgccgtctttgacatcatctgtgtatttttttgtatttttgggtctgtgcacctggtggtcctggtgccggacgttcgacccattgctgttcagctgtctctgtgttctgctggctattttcactgctggacatttgattggactttatttataccagttccaattctttcaagaggcagttccacccaatgactactatgcaaggttgtttggtatcaagtcagtaattcaaacggactgttcaagtacttggaagatcatagtgaacccggacctgtcgtggtaccaccacgccaaccctatcctcctgctggtgatgtactacactctggccactctgatccgcatctggctgcaagagccccttgtgcaggatgaggggaccaaagaagaggacaaagccctggcttgtagccccatccaaataacagcggggaggaggcggagcctgtggtacgcaacccattaccccactgatgagagaaaacttttatccatgacccaggatgactacaaaccatctgatggcctgctggtgactgtgaacggcaaccccgtggattaccacaccatccacccaagcctgcccatggagaacggccctggcaaagccgacctctactccacccctcagtaccggtgggagccctctgatgaatcctcagaaaagcgagaggaggaagaggaagagaaagaagaatttgaagaagaaaggagccgtgaggaaaaaagaagtatcaaagttcatgccatggtctccgtattccaatttattatgaaacaaagttacatctgtgccctcatagctatgatggcctggagcatcacctatcacagctggctgaccttcgtgctgctgatctggtcgtgcactctttggatgattcgcaacagaagaaaatatgccatgatcagctctcccttcatggtggtttatggaaacctattgttgatattacagtatatatggagttttgaacttcctgaaattaaaaaagttccaggatttttagaaaagaaagagccaggagaacttgcttccaaaatccttttcaccattactttttggctactgctgaggcagcacctcacagagcaaaaagctctgcaagaaaaggaagctcttttatcggaagtcaaaattggcagtcaggaaaatgaagaaaaagatgaggaacttcaagatatacaagtggaaggagagcccaaagaggaggaagaagaggaagcgaaggaagagaagcaagagagaaagaaggtagagcaagaggaagctgaagaagaagatgagcaggacatcatgaaagtcctgggcaatctggtggtggccatgttcatcaagtactggatctacgtctgcggaggcatgttcttcttcgtcagcttcgagggtaaaatcgtaatgtacaaaatcatctacatggtgctgttcctgttctgtgtggccctataccaggtgcactatgaatggtggaggaaaattctaaaatatttttggatgtcagtggttatttacactatgctggtgcttatctttatatacacatatcagtttgagaacttcccaggcctgtggcaaaatatgactggactgaaaaaagaaaagcttgaggatcttggcttaaagcagtttactgtggctgaactattcactcgcatattcatcccaacctcctttctgctggtgtgcattttacacctgcactacttccatgaccggttccttgaactcacagacctcaagtccattcccagcaaagaagacaacaccatctacagactggcccacccggaaggaagcctcccggacctcaccatgatgcatctgactgccagcctggagaagccggaggtgaggaagttggctgagcctggggaggagaagcttgagggctactctgaaaaagcccagaagggtgatcttgggaaagacagcgaggagtcagaggaggacggagaggaagaggaggaatccgaggaggaggaagaaacatcagacttaaggaacaaatggcacctggtgattgaccgcctcactgtgctcttcttaaaattcctggagtattttcacaagctgcaggtgttcatgtggtggattttggagttgcacatcatcaaaatcgtttcctcttacattatctgggtttctgtgaaagaggtgtctctgttcaactatgtatttttgatttcttgggcttttgctctgccgtacgccaagctgcgccgtctggcttcaagtgtctgcacagtctggacgtgtgtgatcatcgtctgcaaaatgttgtaccagctccaaaccattaagcctgagaacttctctgttaactgttccttgccaaatgaaaatcaaacaaacatcccctttaatgagttgaacaagtctctgctctacagcgctcctatcgatcctacagagtgggtcggcctgcggaagtcttcgcctctgctagtctacctgaggaataacctcctgatgctggctatcctggcctttgaagtcaccatttaccgccatcaggaatactatcgaggtcgaaataacctgacggcccctgtgtctagaactatctttcatgacattacaagactacatctagatgatggacttattaattgtgccaaatatttcattaattacttcttttacaagtttggtctggagacctgtttcctaatgtcagttaacgtcattggccagcgaatggatttctatgccatgatccacgcctgctggctgatcgctgtcttatatagacgcagaaggaaagccatcgcagagatctggcccaagtactgctgcttcctggcatgcatcatcaccttccagtatttcatctgcattggcatcccacctgctccttgccgagattacccgtggagattcaagggtgccagcttcaatgacaacatcataaagtggctgtacttcccagatttcattgtgcggcccaaccctgtgtttctcgtctatgacttcatgctgcttctgtgtgcctccttacaacggcagatttttgaggatgagaacaaggctgcagtgcgaatcatggcaggtgacaatgtcgagatctgcatgaaccttgatgcggcctccttcagccaacataaccctgtgccagattttattcactgcagatcttacttagacatgtccaaagtgatcatcttcagctacctcttctggtttgtgctcaccatcatcttcatcactgggaccaccaggatcagcatcttttgcatggggtacctggtggcctgtttctacttcctgctctttgggggcgatttgctgttgaaacccatcaagagcatcctgcgctactgggactggctgatcgcatacaacgtttttgtgattacgatgaaaaatatcctgtcaataggagcatgtggatacattggaacattggtgcacaatagttgttggttgatccaggctttcagcctggcctgcacagtcaaaggctatcaaatgcctgctgctaattcaccctgtacacttcccagtggggaagcaggaatcatttgggacagcatatgttttgccttcctcctgctgcaaagaagagttttcatgagttattattttctacatgttgtggctgatataaaagcttcccagattctggcatcaagaggagctgaacttttccaggccacaattgtaaaagctgtaaaggcaagaattgaggaagagaagaagtccatggaccagctgaagcgacagatggatcgcatcaaggccaggcaacagaaatataaaaagggtaaggagaggatgctgagcttgacccaggagccaggggaaggccaggacatgcaaaaactctctgaagaggatgatgaaagagaagcagacaaacagaaagccaagggcaaaaaaaagcagtggtggcggccttgggttgatcatgcttccatggtcaggagtggagattattatttgtttgaaacggatagtgaagaggaggaagaggaagaattaaagaaggaagatgaagaacctccacgaaggtcagcattccagtttgtttatcaagcctggattactgatcctaaaacagcactccgacaaagacacaaagagaaaaaaaggtctgcaagagaagaacggaaacgaaggcggaaaggatccaaggagggtcctgtggaatgggaagaccgggaggatgaaccaatcaaaaagaaatccgatggaccagataatatcatcaagaggatatttaatattttgaaatttacctgggtcctatttctggcaacagtggacagtttcactacttggcttaactccatttcaagggagcatattgatatatctacagttctgagaattgaacgatgcatgctgaccagagaaattaagaagggcaatgttccaactcgggagagcatccacatgtactatcagaaccacatcatgaacctttccagagagtcgggactggacaccattgacgagcatcccggagctgcttcaggtgcacagacagcccacaggatggatagtttagattcacatgacagtatctccagcgagcccacgcagtgtaccatgctgtactcacgccaggggaccactgagaccatcgaggaggtggaggctgagcaggaggaggaggcagggagcacggcgcctgagcccagggaggccaaggagtacgaggccactgggtacgatgtgggagccatgggtgccgaggaggccagcctcaccccagaggaagagctgacacagttctccaccttggacggggatgtggaggccccaccctcctacagcaaggctgtgagcttcgagcatctgtccttcggctcgcaggacgactctgcaggcaagaaccgtatggcagtcagcccggacgacagccgcaccgacaagctggggtccagcatcttacctcccctgacccatgagctgacggccagcgagctgctgctgaaaaagatgtttcacgacgatgagcttgaagagtcagagaaattctacgtggggcagccccgatttctgctgctcttctatgccatgtacaataccctggtggcccgctcggagatggtgtgctacttcgtgatcatcctcaaccacatggtctctgcctccatgatcacgctcctgcttcccatcctcatcttcctctgggccatgttgtccgtccccaggcccagccgccggttctggatgatggccatcgtctatactgaggtggcaattgtagtcaagtatttcttccaatttgggttctttccctggaataagaatgtggaggtgaacaaagataaaccgtatcaccccccaaacatcataggagtggaaaagaaggaaggttatgttctctatgacctcatccagctcctggctctgttctttcatcgatcaattttgaagtgccatggcttatgggatgaagatgacatgactgaaagtggcatggccagggaggaatcagatgatgagctctccctcggtcatggcaggagggactcctccgattctctcaagtccatcaacctggccgcgtctgtggagtcagtgcatgtgaccttcccggagcagcagacagctgtccggaggaagcgctccggcagcagctccgagccatcccagagatccagcttttcttcaaacagatcccaaagaggcagcacaagcacccgaaacagcagtcaaaaaggaagcagtgttttgagtattaagcaaaaaggcaaaagggaactttatatggaaaagcttcaagaacatttaatcaaagcaaaagcctttaccataaagaagacgctggagatctatgtgcccatcaaacagttcttttacaacctcatccacccggagtatagcgccgtgactgacgtgtatgtactcatgttcctggctgacactgtggacttcatcatcattgtcttcggcttttgggcctttgggaaacactcagcagctgcagacatcacctcttcactgtcagaggaccaggtcccggggccgtttttggtgatggtcctcattcagtttggaaccatggtggtggaccgagccctctacctcaggaagactgtactgggaaaggtcatcttccaggtcattcttgtgttcggaattcacttctggatgttcttcatcttacctggtgtgactgagaggaaattcagccagaacctggttgcccagctttggtactttgtgaaatgtgtttacttcgggttgtctgcttaccagatccgttgtggctacccaacgcgagtcctggggaacttcctcaccaagagctacaattacgtcaacctcttcttattccaagggtttcgcctcgtgccctttttgactgagctgagggcagtgatggactgggtgtggacggacacaactttgagcctgtccagctggatctgtgtggaggacatctatgctcacatattcatcctgaagtgttggcgggagtcggagaagagataccctcagccacggggccagaagaagaagaaagtggtgaagtatggcatgggaggaatgatcatcgtcctgctcatctgcattgtctggtttcctcttctcttcatgtctttgatcaaatctgtggctggggtcatcaaccagcccctggacgtctccgtcacaattaccctgggagggtatcagcctattttcacaatgagtgcccaacaaagccagttgaaagttatggaccagcagagctttaacaaatttatacaagctttttctagggacaccggtgctatgcaatttctggaaaattatgaaaaagaagacataacagtagcagaactggaaggaaactcaaattctttgtggaccatcagcccacccagtaagcagaaaatgatacacgaactcctggaccccaatagtagcttctctgttgttttttcatggagtattcagagaaacttaagtctgggtgcaaaatcggaaatagcaacagataagctttcttttcctcttaaaaatattactcgaaagaatatcgctaaaatgatagcaggcaacagcacagaaagttcaaaaacaccagtgaccatagaaaagatttatccatattatgtgaaagcacctagtgattctaactcaaaacctataaagcaacttttatctgaaaataatttcatggatattaccatcattttgtccagagacaatacaactaaatataacagtgagtggtgggttctcaacctgactggaaacagaatatacaatccgaactctcaggccctggaactggtggtcttcaatgacaaagtcagtcccccaagtctggggttcctggctggctatggtattatgggattatatgcttcagttgtccttgtgattgggaaatttgtccgtgaattcttcagtgggatttctcactccatcatgtttgaagagcttccaaatgtggatcgaattttgaagttgtgcacagatatttttttagttcgagagacaggagaactggagctagaagaagatctctatgccaaattaatattcctatatcgctcaccagagacaatgatcaaatggactagagaaaaaacaaattga

SEQ ID NO: 20 - protein, 2752 aa, Homo sapiens
MASEVVCGLIFRLLLPICLAVACAFRYNGLSFVYLIYLLLIPLFSEPTKTTMQGHTGRLLKSLCFISLSFLLLHIIFHITLVSLEAQHRIAPGYNCSTWEKTFRQIGFESLKGADAGNGIRVFVPDIGMFIASLTIWLLCRNIVQKPVTDEAAQSNPEFENEELAEGEKIDSEEALIYEEDFNGGDGVEGELEESTKLKMFRRLASVASKLKEFIGNMITTAGKVVVTILLGSSGMMLPSLTSSVYFFVFLGLCTWWSWCRTFDPLLFSCLCVLLAIFTAGHLIGLYLYQFQFFQEAVPPNDYYARLFGIKSVIQTDCSSTWKIIVNPDLSWYHHANPILLLVMYYTLATLIRIWLQEPLVQDEGTKEEDKALACSPIQITAGRRRSLWYATHYPTDERKLLSMTQDDYKPSDGLLVTVNGNPVDYHTIHPSLPMENGPGKADLYSTPQYRWEPSDESSEKREEEEEEKEEFEEERSREEKRSIKVHAMVSVFQFIMKQSYICALIAMMAWSITYHSWLTFVLLIWSCTLWMIRNRRKYAMISSPFMVVYGNLLLILQYIWSFELPEIKKVPGFLEKKEPGELASKILFTITFWLLLRQHLTEQKALQEKEALLSEVKIGSQENEEKDEELQDIQVEGEPKEEEEEEAKEEKQERKKVEQEEAEEEDEQDIMKVLGNLVVAMFIKYWIYVCGGMFFFVSFEGKIVMYKIIYMVLFLFCVALYQVHYEWWRKILKYFWMSVVIYTMLVLIFIYTYQFENFPGLWQNMTGLKKEKLEDLGLKQFTVAELFTRIFIPTSFLLVCILHLHYFHDRFLELTDLKSIPSKEDNTIYRLAHPEGSLPDLTMMHLTASLEKPEVRKLAEPGEEKLEGYSEKAQKGDLGKDSEESEEDGEEEEESEEEEETSDLRNKWHLVIDRLTVLFLKFLEYFHKLQVFMWWILELHIIKIVSSYIIWVSVKEVSLFNYVFLISWAFALPYAKLRRLASSVCTVWTCVIIVCKMLYQLQTIKPENFSVNCSLPNENQTNIPFNELNKSLLYSAPIDPTEWVGLRKSSPLLVYLRNNLLMLAILAFEVTIYRHQEYYRGRNNLTAPVSRTIFHDITRLHLDDGLINCAKYFINYFFYKFGLETCFLMSVNVIGQRMDFYAMIHACWLIAVLYRRRRKAIAEIWPKYCCFLACIITFQYFICIGIPPAPCRDYPWRFKGASFNDNIIKWLYFPDFIVRPNPVFLVYDFMLLLCASLQRQIFEDENKAAVRIMAGDNVEICMNLDAASFSQHNPVPDFIHCRSYLDMSKVIIFSYLFWFVLTIIFITGTTRISIFCMGYLVACFYFLLFGGDLLLKPIKSILRYWDWLIAYNVFVITMKNILSIGACGYIGTLVHNSCWLIQAFSLACTVKGYQMPAANSPCTLPSGEAGIIWDSICFAFLLLQRRVFMSYYFLHVVADIKASQILASRGAELFQATIVKAVKARIEEEKKSMDQLKRQMDRIKARQQKYKKGKERMLSLTQEPGEGQDMQKLSEEDDEREADKQKAKGKKKQWWRPWVDHASMVRSGDYYLFETDSEEEEEEELKKEDEEPPRRSAFQFVYQAWITDPKTALRQRHKEKKRSAREERKRRRKGSKEGPVEWEDREDEPIKKKSDGPDNIIKRIFNILKFTWVLFLATVDSFTTWLNSISREHIDISTVLRIERCMLTREIKKGNVPTRESIHMYYQNHIMNLSRESGLDTIDEHPGAASGAQTAHRMDSLDSHDSISSEPTQCTMLYSRQGTTETIEEVEAEQEEEAGSTAPEPREAKEYEATGYDVGAMGAEEASLTPEEELTQFSTLDGDVEAPPSYSKAVSFEHLSFGSQDDSAGKNRMAVSPDDSRTDKLGSSILPPLTHELTASELLLKKMFHDDELEESEKFYVGQPRFLLLFYAMYNTLVARSEMVCYFVIILNHMVSASMITLLLPILIFLWAMLSVPRPSRRFWMMAIVYTEVAIVVKYFFQFGFFPWNKNVEVNKDKPYHPPNIIGVEKKEGYVLYDLIQLLALFFHRSILKCHGLWDEDDMTESGMAREESDDELSLGHGRRDSSDSLKSINLAASVESVHVTFPEQQTAVRRKRSGSSSEPSQRSSFSSNRSQRGSTSTRNSSQKGSSVLSIKQKGKRELYMEKLQEHLIKAKAFTIKKTLEIYVPIKQFFYNLIHPEYSAVTDVYVLMFLADTVDFIIIVFGFWAFGKHSAAADITSSLSEDQVPGPFLVMVLIQFGTMVVDRALYLRKTVLGKVIFQVILVFGIHFWMFFILPGVTERKFSQNLVAQLWYFVKCVYFGLSAYQIRCGYPTRVLGNFLTKSYNYVNLFLFQGFRLVPFLTELRAVMDWVWTDTTLSLSSWICVEDIYAHIFILKCWRESEKRYPQPRGQKKKKVVKYGMGGMIIVLLICIVWFPLLFMSLIKSVAGVINQPLDVSVTITLGGYQPIFTMSAQQSQLKVMDQQSFNKFIQAFSRDTGAMQFLENYEKEDITVAELEGNSNSLWTISPPSKQKMIHELLDPNSSFSVVFSWSIQRNLSLGAKSEIATDKLSFPLKNITRKNIAKMIAGNSTESSKTPVTIEKIYPYYVKAPSDSNSKPIKQLLSENNFMDITIILSRDNTTKYNSEWWVLNLTGNRIYNPNSQALELVVFNDKVSPPSLGFLAGYGIMGLYASVVLVIGKFVREFFSGISHSIMFEELPNVDRILKLCTDIFLVRETGELELEEDLYAKLIFLYRSPETMIKWTREKTN

SEQ ID NO: 21 - DNA, 18 bp, Artificial Sequence
  Mus musculus primer
atggagccgcacgtgctg

SEQ ID NO: 22 - DNA, 21 bp, Artificial Sequence
  Mus musculus primer
ctactccctctcacgtgtcca

SEQ ID NO: 23 - DNA, 21 bp, Artificial Sequence
  Mus musculus primer
atggcttcggaagtggtgtgc

SEQ ID NO: 24 - DNA, 25 bp, Artificial Sequence
  Mus musculus primer
tcagtttgttttttctctagtccac

(19) United States
(12) Patent Application Publication
(10) Pub. No.: US 2013/0156762 A1
(43) Pub. Date: Jun. 20, 2013

(54) Mechanically-Activated Cation Channels

(71) Applicants: Bertrand Coste, Pertuis (FR); Ardem Patapoutian, Del Mar, CA (US)

(72) Inventors: Bertrand Coste, Pertuis (FR); Ardem Patapoutian, Del Mar, CA (US)

(73) Assignees: The Scripps Research Institute, La Jolla, CA (US); IRM LLC, Hamilton (BM)

(21) Appl. No.: 13/818,609

(22) PCT Filed: Aug. 23, 2011
(86) PCT No.: PCT/US11/48836

§ 371 Date: Feb. 22, 2013

Related U.S. Application Data
(60) Provisional application No. 61/376,182, filed on Aug. 23, 2010.

(51) Int. Cl. C07K 16/18; C07H 21/00

(52) U.S. Cl. CPC C07K 16/18; C07H 21/00
USPC 424/133.1; 424/139.1; 435/29; 435/325; 435/320.1; 514/44 A; 530/387.3; 530/387.9; 536/24.5

(57) ABSTRACT

Methods of screening for agents that modulate the activity of a mechanically-activated cation channel are provided. Also provided are compositions and methods for ameliorating pain by antagonizing or inhibiting mechanically-activated cation channels.

### CROSS-REFERENCE TO RELATED APPLICATION

This application claims the benefit of U.S. Provisional Application No. 61/376,182, filed Aug. 23, 2010. This priority application is hereby incorporated by reference in its entirety.

### STATEMENT AS TO RIGHTS TO INVENTIONS MADE UNDER FEDERALLY SPONSORED RESEARCH AND DEVELOPMENT

This invention was made with Government support under Grant No. DE016927 and NS046303 awarded by the National Institutes of Health. The government has certain rights in this invention.

### BACKGROUND OF THE INVENTION

Mechanotransduction, the conversion of mechanical force into biological signals, has crucial roles in physiology. In mammals, embryonic development, touch, pain, proprioception, hearing, adjustment of vascular tone and blood flow, flow sensing in kidney, lung growth and injury, bone and muscle homeostasis as well as metastasis are all regulated by mechanotransduction (M. Chalfie, Nat. Rev. Mol. Cell. Biol. 10, 44 (January 2009); O. P. Hamill, B. Martinac, Physiol Rev 81, 685 (April 2001)). In plants, mechanical force strongly impacts morphogenesis, for example in lateral root formation (G. B. Monshausen, S. Gilroy, Trends Cell Biol. 19, 228 (May 2009). Even unicellular organisms such as ciliates sense touch and change direction in response to a tactile stimulus (K. Iwatsuki, T. Hirano, Comp. Biochem. Physiol. A. Physiol. 110, 167 (February 1995)).

Electrophysiological recordings from vertebrate inner ear hair cells show that mechanotransduction is extremely rapid, implicating an ion channel directly activated by force D. P. Corey, A. J. Hudspeth, Biophys. J. 26, 499 (June 1979). Indeed, calcium-permeable mechanically-activated (MA) cationic currents have been described in various mechanosensitive cells, including in dorsal root ganglion (DRG) neurons (G. C. McCarter, D. B. Reichling, J. D. Levine, Neurosci. Lett. 273, 179), in kidney primary cilium (H. A. Praetorius, K. R. Spring, J Membr Biol 184, 71 (Nov. 1, 2001)), and in plants (G. B. Monshausen, S. Gilroy, Trends Cell Biol. 19, 228 (May 2009)). However, few MA channels have been identified to date.

### BRIEF SUMMARY OF THE INVENTION

The present invention provides methods of screening for an agent that modulates the activity of a mechanically-activated cation channel. In some embodiments, the method comprises: contacting a mechanically-activated cation channel polypeptide having at least 70% amino acid sequence identity to one of SEQ ID NOs:2, 4, 18, or 20 with an agent; and selecting the agent that modulates the activity of the mechanically-activated cation channel polypeptide.

In some embodiments, the polypeptide is expressed in a cell and the contacting comprises contacting the cell with the agent. In some embodiments, the polypeptide is heterologous to the cell. In some embodiments, the cell comprises a heterologous expression cassette comprising a promoter operably linked to a polynucleotide encoding the mechanically-activated cation channel polypeptide. In some embodiments, the polynucleotide comprises SEQ ID NO:1, SEQ ID NO:3, SEQ ID NO:17, or SEQ ID NO:19. In other embodiments, the polypeptide is endogenous to the cell. In some embodiments, the cell is a eukaryotic cell. In some embodiments, the cell is a neuron.

In some embodiments, the activity of the mechanically-activated cation channel polypeptide is determined by measuring an electrophysiological change mediated by the polypeptide. In certain embodiments, the electrophysiological change is a change in membrane potential, a change in current, or an influx of a cation. In some embodiments, the membrane potential is measured with a membrane potential dye assay. In some embodiments, the electrophysiological change is measured with a patch-clamp assay. In certain embodiments, the measuring comprises measuring a mechanically-activated electrophysiological change.

In some embodiments, the method further comprises testing an agent identified as modulating the activity of the mechanically-activated cation channel polypeptide for the ability to modulate a mechanically-activated electrophysiological change.

In some embodiments, the selected agent reduces or inhibits the electrophysiological change mediated by the polypeptide. In some embodiments, the selected agent increases the electrophysiological change mediated by the polypeptide.

In some embodiments, the cell is in an animal. In some embodiments, the animal is a mouse. In some embodiments, the method further comprises administering the agent to the animal and determining the effect of the agent on pain sensitivity.

In some embodiments, the polypeptide comprises SEQ ID NO:2, SEQ ID NO:4, SEQ ID NO:18, or SEQ ID NO:20.

The present invention also provides antibodies that antagonize the activity of a mechanically-activated cation channel.

In some embodiments, the antibody selectively binds to a mechanically-activated cation channel polypeptide having at least 70% amino acid sequence identity to SEQ ID NO:2, SEQ ID NO:4, SEQ ID NO:18, or SEQ ID NO:20. In certain embodiments, the polypeptide comprises SEQ ID NO:2, SEQ ID NO:4, SEQ ID NO:18, or SEQ ID NO:20.

In some embodiments, the antibody is a monoclonal antibody. In certain embodiments, the antibody is a chimeric antibody. In other embodiments, the antibody is a humanized antibody.

The present invention also provides methods of ameliorating pain in a subject. In some embodiments, the method comprises administering to the subject an antibody that selectively binds to a mechanically-activated cation channel polypeptide having at least 70% amino acid sequence identity to SEQ ID NO:2, SEQ ID NO:4, SEQ ID NO:18, or SEQ ID NO:20.

In some embodiments, the method comprises administering an antibody that selectively binds to a mechanically-activated cation channel polypeptide comprising SEQ ID NO:4 or SEQ ID NO:20. In some embodiments, the polypeptide is expressed in bladder, colon, kidney, lung, or skin. In some embodiments, the polypeptide is expressed in a dorsal root ganglion neuron.

In some embodiments, the subject is a mammal. In certain embodiments, the subject is a human.

In some embodiments, the pain is selected from the group consisting of acute mechanical pain, chronic mechanical pain, mechanical hyperalgesia, mechanical allodynia, arthritis, inflammation, dental pain, cancer pain, and labor pain.

The present invention also provides isolated antisense oligonucleotides or small interfering RNAs (siRNAs) complementary to at least 15 contiguous nucleotides of a polynucleotide that is at least 70% identical to SEQ ID NOs:1, 3, 17, or 19 and encoding a mechanically-activated cation channel polypeptide, wherein the antisense oligonucleotide or siRNA inhibits production of the mechanically-activated cation channel polypeptide.

In some embodiments, the antisense oligonucleotide or small interfering RNA (siRNA) is complementary to at least 15 contiguous nucleotides of SEQ ID NOs: 1, 3, 17, or 19. In some embodiments, the antisense oligonucleotide or siRNA comprises any of SEQ ID NOs:5-16.

The present invention also provides expression cassettes comprising a promoter operably linked to a polynucleotide comprising the antisense oligonucleotide or siRNA complementary to at least 15 contiguous nucleotides of a polynucleotide that is at least 70% identical to SEQ ID NOs:1, 3, 17, or 19 and encoding a mechanically-activated cation channel polypeptide, wherein the antisense oligonucleotide or siRNA inhibits production of the mechanically-activated cation channel polypeptide. The present invention also provides vectors comprising said expression cassettes and cells comprising said expression cassettes and/or said vectors.

The present invention also provides methods of ameliorating pain in a subject, the method comprising administering to the subject an antisense oligonucleotide or small interfering RNA (siRNA) complementary to at least 15 contiguous nucleotides of a polynucleotide that is at least 70% identical to SEQ ID NOs:1, 3, 17, or 19 and encoding a mechanically-activated cation channel polypeptide, wherein the antisense oligonucleotide or siRNA inhibits production of the mechanically-activated cation channel polypeptide.

In some embodiments, the antisense oligonucleotide or siRNA inhibits production of the mechanically-activated cation channel in bladder, colon, kidney, lung, or skin. In certain embodiments, the antisense oligonucleotide or siRNA inhibits production of the mechanically-activated cation channel in a dorsal root ganglion neuron.

In some embodiments, the subject is a mammal. In certain embodiments, the subject is a human.

In some embodiments, the pain is selected from the group consisting of acute mechanical pain, chronic mechanical pain, mechanical hyperalgesia, mechanical allodynia, arthritis, inflammation, dental pain, cancer pain, and labor pain.

### DEFINITIONS

As used herein, the following terms have the meanings ascribed to them unless specified otherwise.

The term “mechanically-activated cation channel” refers to an ion channel that opens to allow passage of positively charged ions (i.e. cations) into and out of a cell in response to mechanical force or pressure being applied, e.g., to a cell expressing the channel. As used herein, the term also includes polypeptide components of mechanically-activated cation channels, e.g., subunits of a cation channel. In some embodiments, the mechanically-activated cation channels of the present invention are substantially identical to SEQ ID NO:2, SEQ ID NO:4, SEQ ID NO:18, or SEQ ID NO:20. In some embodiments, the mechanically-activated cation channels of the present invention are involved in sensory transduction, such as pain transduction, including but not limited to, cells such as neurons.

“Inhibitors,” “activators,” and “modulators” of mechanically-activated cation channel polypeptide activity are used interchangeably herein to refer to inhibitory, activating, or modulating molecules identified using in vitro and in vivo assays for sensory (e.g., pain or somatosensory) transduction, e.g., ligands, agonists, antagonists, and their homologs and mimetics. The term “modulator” encompasses inhibitors and activators. Inhibitors are compounds that, e.g., bind to, partially or totally block stimulation, decrease, prevent, delay activation, inactivate, desensitize, or down regulate signal transduction, e.g., antagonists. Activators are compounds that, e.g., bind to, stimulate, increase, open, activate, facilitate, enhance activation, sensitize, or up regulate signal transduction, e.g., agonists. Modulators include naturally occurring and synthetic ligands, antagonists, agonists, small chemical molecules and the like. Such assays for inhibitors and activators include, e.g., expressing a mechanically-activated cation channel polypeptide in cells or cell membranes, applying putative modulator compounds, and then determining the functional effects on ion flux, membrane potential, electrophysiology, or mechanical activation. Samples or assays comprising a mechanically-activated cation channel polypeptide that is treated with a potential activator, inhibitor, or modulator are compared to control samples without the inhibitor, activator, or modulator to examine the extent of modulation. Control samples (untreated with inhibitors) are assigned a relative mechanically-activated cation channel polypeptide activity value of 100%. Inhibition of a mechanically-activated cation channel polypeptide is achieved when the mechanically-activated cation channel polypeptide activity value relative to the control is about 80%, optionally 75%, 50%, or 25-0%. Activation of the mechanically-activated cation channel polypeptide is achieved when the mechanically-activated cation channel polypeptide activity value relative to the control is 110%, optionally 125%, optionally 150%, optionally 200-500%, or 1000-3000% higher.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term “nucleic acid” encompasses the terms gene, cDNA, mRNA, oligonucleotide, and polynucleotide.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, .gamma.-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG, and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

8) Cysteine (C), Methionine (M) (see, e.g., Creighton, Proteins (1984)).

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. The term “purified” denotes that a nucleic acid or protein gives rise to essentially one band in an electrophoretic gel. Particularly, it means that the nucleic acid or protein is at least 85% pure, optionally at least 95% pure, and optionally at least 99% pure.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed, or not expressed at all.

The term “heterologous” when used with reference to a protein's or nucleic acid's relationship to a cell indicates that the protein or nucleic acid is not found in the same relationship to the cell (e.g., not expressed in the cell) in nature. The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression cassette” is a nucleic acid construct, generated recombinantly or synthetically, with a series of specified nucleic acid elements that permit transcription of a particular nucleic acid in a host cell. The expression cassette can be part of a plasmid, virus, or nucleic acid fragment. Typically, the expression cassette includes a nucleic acid to be transcribed operably linked to a promoter.

The terms “identical” or percent “identity,” in the context of two or more nucleic acids or polypeptide sequences, refer to two or more sequences or subsequences that are the same. Sequences are “substantially identical” of they have a specified percentage of amino acid residues or nucleotides that are the same (i.e., 70% identity, optionally 75%, 80%, 85%, 90%, or 95% identity over a specified region), when compared and aligned for maximum correspondence over a comparison window, or designated region (a specified length, or when not specified, the entire length) as measured using one of the following sequence comparison algorithms or by manual alignment and visual inspection. The present invention provides sequences substantially identical to, e.g., SEQ ID NO:1, SEQ ID NO:2, SEQ ID NO:3, SEQ ID NO:4, SEQ ID NO:17, SEQ ID NO:18, SEQ ID NO:19, or SEQ ID NO:20. Optionally, the identity exists over a region that is at least about 15 amino acids or nucleotides in length, or over a region that is at least about 18 amino acids or nucleotides in length, about 20 amino acids or nucleotides in length, about 22 amino acids or nucleotides in length, about 25-50 amino acids or nucleotides in length, or about 75-100 amino acids or nucleotides in length or more.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., CURRENT PROTOCOLS IN MOLECULAR BIOLOGY (Ausubel et al., eds. 1995 supplement)).

Two examples of algorithms that are suitable for determining percent sequence identity and sequence similarity are the BLAST and BLAST 2.0 algorithms, which are described in Altschul et al.; Nuc. Acids Res. 25:3389-3402 (1977) and Altschul et al., J. Mol. Biol. 215:403-410 (1990), respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see FUNDAMENTAL IMMUNOLOGY (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

A “humanized antibody” is an antibody that retains the reactivity of a non-human antibody while being less immunogenic in humans. This can be achieved, for instance, by retaining the non-human CDR regions and replacing the remaining parts of the antibody with their human counterparts. See, e.g., Morrison et al., Proc. Natl. Acad. Sci. USA, 81:6851-6855 (1984); Morrison and Oi, Adv. Immunol., 44:65-92 (1988); Verhoeyen et al., Science, 239:1534-1536 (1988); Padlan, Molec. Immun., 28:489-498 (1991); Padlan, Molec. Immun., 31(3):169-217 (1994).

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to Piezo1 or Piezo2 from specific species such as rat, mouse, or human can be selected to obtain only those polyclonal antibodies that are specifically immunoreactive with Piezo1 or Piezo2 and not with other proteins, except for polymorphic variants and alleles of Piezo1 or Piezo2. This selection may be achieved by subtracting out antibodies that cross-react with Piezo1 or Piezo2 molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

A “subject” or “individual” refers to an animal, including a human, non-human primate, mouse, rat, rabbit, dog, or other mammal.

### BRIEF DESCRIPTION OF THE DRAWINGS

FIG. 1. Neuro2A and C2C12 cells display different types of mechanically-activated currents. (A, B) Representative traces of mechanically-activated (MA) inward currents expressed in Neuro2A (N2A, A) and C2C12 (B) cells. The cells were subjected to a series of mechanical steps in 1 μm increments using a stimulation pipette (inset drawing, arrow) in the whole-cell patch configuration at a holding potential of −80 mV. (C) Ratio of inactivated current at the end of a mechanical step (150 ms duration) relative to the peak current (mean±SEM, number of cells above the bars) in N2A and C2C12 cells for currents elicited at a holding potential of −80 mV. ***, P<0.001. (D, E) Average current-voltage relationships of MA currents in N2A (D, n=11) and C2C12 (E, n=4) cells. Inset, representative MA currents evoked at holding potentials ranging from −80 to +40 mV (applied 0.7 sec prior to the mechanical step). (F) Average maximal amplitude of MA inward currents elicited in N2A and C2C12 cells at a holding potential of −80 mV (mean±SEM, the number of cells tested is shown above the bars). *, P<0.05. (G) Single-channel currents (cell attached patch configuration) induced by negative pipette pressure (inset drawing, arrow) at holding potentials ranging from −80 mV to +80 mV in a N2A cell. (H) Average current-voltage relationships of stretch-activated single channels in N2A cells (n=4, mean±SEM). Single channel conductance is calculated from the slope of the linear regression line of each cell giving γ=22.9±1.4 pS (mean±SEM). Single channel amplitude was determined as the amplitude difference in Gaussian fits of full trace histograms. (I) Representative currents (averaged traces) induced by negative pipette pressure (0 to −60 mm Hg, Δ 10 mm Hg) in a N2A cell. (J) Normalized current-pressure relationship of stretch activated currents at −80 mV fitted with a Boltzmann equation (n=21). P₅₀ is the average value of P₅₀s from individual cells.

FIG. 2. Suppression of mechanically-activated currents by Piezo1 (Fam38A) siRNA. (A) Average maximal amplitude of MA inward currents elicited at a holding potential of −80 mV in N2A cells transfected with scrambled siRNA (far left dot, n=56), Piezo1 (Fam38A) siRNA (bottom right dot, n=20) or siRNA directed against other candidates tested (black dots, list of candidates available as Table S2). For each candidate, black circle and error bar represents the mean±SEM, n=4-27 each. The black line represents the average value of all cells tested (n=807), and the two dashed lines represent 4-fold decrease or increase of this value. (B) Representative traces of MA inward currents expressed in N2A cells transfected with scrambled siRNA (top trace) or Piezo1 (Fam38A) siRNA (bottom trace) at a holding potential of −80 mV. (C) Average maximal amplitude of MA inward currents elicited at a holding potential of −80 mV in N2A cells transfected either with scrambled siRNA (left bar) or different Piezo1 (Fam38A) siRNAs. siRNA 1, 2, and 3 are siRNAs of smart-pool I tested individually. (D) Representative currents (averaged traces) induced by negative pipette pressure (0 to −60 mm Hg, Δ 10 mm Hg, cell attached) in a N2A cell transfected with scrambled siRNA (left panel) or Piezo1 siRNA (right panel). Traces of current elicited by −60 mm Hg are highlighted. (E) Average maximal amplitude of stretch-activated currents elicited at a holding potential of −80 mV in N2A cells transfected with scrambled siRNA (left bar) or Piezo1 siRNA (right bar). Bars represent the mean±SEM, and the number of cells tested is shown above the bars. **, P<0.01. ***, P<0.001.

FIG. 3. Piezo1 siRNA qPCR and cell viability control, and N2A MA currents after disruption of integrin function. (A) siRNA-induced down-regulation of Piezo1 mRNA in N2A cells. Transfected and untransfected cells are unsorted and thus these differences are underestimated. (B) Representative ratiometric calcium imaging experiment of capsaicin stimulated N2A cells co-transfected with TRPV 1 and GFP, together with either scrambled siRNA or Piezo1 siRNA (mean±SEM of GFP-positive cell traces). (C) Percentage of GFP-positive cells responding to capsaicin (left panel, mean±SEM of two experiments) and Fura-2 340/380 ratio fold change of capsaicin responding cells (mean±SEM). (D) Maximal current amplitude of whole cell MA currents elicited in N2A cells at a holding potential of −80 mV in control conditions or after 30-60 minutes perfusion with a divalent free solution containing 5 mM EGTA. (E) Representative traces of MA currents normalized to peak in control conditions or after 30-60 minutes perfusion with a divalent free solution containing 5 mM EGTA (left panel). Inactivation of currents is fitted with a mono-exponential equation. In the absence of divalent cations, the time constant for inactivation is higher than with control solution (right panel). ***, P<0.001, unpaired t-test.

FIG. 4. Evolutionary conservation, hydrophobicity plot, and expression profile of Piezo1 and Piezo2. (A) Unrooted phylogenetic tree showing sequence relationship of different members of the Piezo family of proteins. The alignments were generated using Megalign and DrawTree programs. The dotted line represents an artificially extended line to accommodate fit. Hs, Homo Sapiens; Mm, Mouse musculus; Gg, Gallus gallus, Dr, Danio Rerio; Ci, Ciona intestinalis; Dm, Drosophila melanogaster; Ce, Caenorhabditis elegans; Dd, Dictyostelium discoideum; At, Arabidopsis thaliana; Os, Oryza sativa; Tt, Tetrahymena thermophila (accession numbers are provided in Methods). (B) Hydrophobicity analysis of mouse Piezo1. The Kyte-Doolittle pattern (19 residues window) shows succession of hydrophobic and hydrophilic regions. 30 transmembrane domains are predicted by TMHMM2. (C) mRNA expression profiles of Piezo1 (left panel) and Piezo2 (right panel) determined by qPCR from various adult mouse tissues. GAPDH was used as the reference gene and lung as the tissue calibrator using the 2^{-ΔΔCT} method. Each bar is the mean±SEM of the average of two separate experiments.

FIG. 5. Piezo1 induces large, mechanically-activated nonselective cationic currents. (A-F) MA currents of Piezo1-expressing N2A (A-C) and HEK293T (D-F) cells recorded in the whole-cell configuration. (A, D) Representative traces of MA inward currents expressed in different cell types transfected with Piezo1. The cell was subjected to a series of mechanical steps in 1 μm (A) or 0.5 μm (D) increments using glass probe stimulation and at a holding potential of −80 mV. (B, E) Representative current-voltage relationships of MA currents expressed in different cell types transfected with Piezo1. Inset, MA currents evoked at holding potentials ranging from −80 to +40 mV. (C, F) Average maximal amplitude of MA inward currents elicited at a holding potential of −80 mV in Piezo1-transfected (right bar) or mock-transfected (left bar) cells. Bars represent the mean±SEM, and the number of cells tested is shown above the bars. ***, P<0.001. (G) P_{X}/P_{Cs} ion selectivity ratios of MA currents in Piezo1-expressing cells. (H) Percent block of MA currents in Piezo1-expressing cells by 30 μM gadolinium or ruthenium red. (I-N) Stretch-activated currents of mouse Piezo1-expressing N2A (I-K) and HEK293T (L-N) cells in cell-attached configuration. Representative averaged currents induced by negative pipette pressure (0 to −60 mm Hg, A 10 mm Hg) in N2A (I) and HEK293T (L) cells transfected with Piezo1. Normalized current-pressure relationship of stretch-activated currents elicited at −80 mV in Piezo1-transfected N2A (J, n=12) and HEK293T (M, n=11) cells and fitted with a Boltzmann equation. P₅₀ is the average value of all P₅₀s determined for individual cells. Average maximal amplitude of stretch-activated currents elicited at a holding potential of −80 mV in N2A (K) and HEK293T (N) cells mock-transfected (left bar) or transfected with Piezo1 (right bar). Bars represent the mean±SEM, and the number of cells tested is shown above the bars. ***, P<0.001. **, P<0.01.

FIG. 6. Piezo1-induced MA currents are cationic non-selective currents blocked by gadolinium and ruthenium red. (A-C) MA currents of Piezo1-expressing C2C12 cells recorded in the whole-cell configuration. (A) Representative traces of MA inward currents expressed in Piezo1-transfected cells. The cell is subjected to a series of mechanical steps in 1 μm increments using glass probe stimulation and at a holding potential of −80 mV. (B) Representative current-voltage relationships of MA currents expressed in Piezo1-transfected cells. Inset, MA currents evoked at holding potentials ranging from −80 to +40 mV. (C) Average maximal amplitude of MA inward currents elicited at a holding potential of −80 mV in Piezo1-transfected (right bar) or mock-transfected (left bar) cells. Bars represent the mean±SEM, and the number of cells tested is shown above the bars. ***, P<0.001, unpaired t-test with Welch's correction. (D) Whole-cell MA current traces elicited in a Piezo1 transfected cell bathed in control solution (left panel) or after perfusion with 150 mM NMDG-Cl (middle panel) solution. Currents are elicited from −80 mV to +80 mV in 40 mV steps. Right panel shows the MA current-voltage relationship from the same cell. Note that inward currents present in control condition (filled symbols) are suppressed with external NMDG-Cl solution (open symbol). (E-F) Average current-voltage relationship of MA currents elicited in Piezo1 transfected HEK293T cells and recorded with CsCl-based internal solution and 150 mM NaCl—, 150 mM KCl—, 100 mM CaCl₂- or 100 mM MgCl₂-based extracellular solutions. (E) I-V relationships from individual cells were normalized to the value at −40 mV before liquid junction potentials were corrected. (F) Average of reversal potential values determined for each recording conditions and for individual cells (mean±SEM). (G-H) Representative current traces of MA currents elicited in Piezo1 transfected cells before, during and after perfusion of 30 μM gadolinium (E) or ruthenium red (F).

FIG. 7. Piezo2 induces large mechanically-activated currents kinetically distinct from Piezo1-induced currents. (A-F) MA currents of Piezo2-expressing N2A (A-C) and HEK293T (D-F) cells in whole-cell configuration. In N2A cells, Piezo2 or vector only were co-transfected with Piezo1 siRNA to suppress endogenous Piezo1-dependent MA currents. (A, D) Representative traces of MA inward currents expressed in different cell types transfected with Piezo2. The cell was subjected to a series of mechanical steps in 1 μm increments using glass probe stimulation at a holding potential of −80 mV. (B, E) Representative current-voltage relationships of MA currents expressed in different cell types transfected with Piezo2. Inset, MA currents evoked at holding potentials ranging from −80 to +40 mV. (C, F) Average maximal amplitude of MA inward currents elicited at a holding potential of −80 mV in Piezo1-transfected (right bar) or mock-transfected (left bar) cells. (G-H) Representative traces of mechanically-activated inward (G) or outward (H) currents expressed in cells transfected with Piezo1 (right trace) or Piezo2 (left trace) at the specified holding potentials. Traces are normalized to peak, and dashed lines represent fits of inactivation with a mono-exponential equation. (I) Time-constant of inactivation of Piezo1 (left bar) and Piezo2 (right bar) at negative (−80 and −40 mV, upper panel) and positive (40 and 80 mV, lower panel) holding potentials. Bars represent the mean±SEM and the numbers above bars the number of cells. **, P<0.01. ***, P<0.001.

FIG. 8. Piezo2-induced MA currents are cationic non-selective. (A) Whole-cell MA current traces elicited in a Piezo2 transfected cell bathed in control solution (left panel) and after perfusion with NMDG-Cl (right panel) solution. Currents are elicited at −80, −40, 0, +40 and +80 mV. (B) MA current-voltage relationships from the same cell. Note that inward currents present in control condition (filled symbols) were suppressed with NMDG-Cl solution (open symbol). (C) Representative current traces of MA currents elicited in Piezo2-transfected cells before, during and after perfusion of 30 μM gadolinium (upper panels) or ruthenium red (lower panels). (D) Percent block of MA currents in Piezo2-expressing cells by 30 μM gadolinium and ruthenium red. Bars represent the mean±SEM, and the number of cells tested is shown above the bars.

FIG. 9. Piezo1 antibodies detect Piezo1 in transfected HEK293T cells. (A) Representative images of Piezo1 labeling (red) in Piezo1-IRES-EGFP transfected cells (green). Note, GFP-negative, hence untransfected, cells are devoid of labeling. (B) A proportion of Piezo1 is expressed near or at the plasma membrane of TRPA1 and Piezo1 co-transfected HEK293T cells. Cells were live-labeled with TRPA1 antibodies (green) to delineate the plasma membrane, fixed, permeabilized, and stained for Piezo1 (red) and MYC (total TRPA1). Inset, higher magnification of boxed area in overlay image. Scale bars=20 μm.

FIG. 10. siRNA-knockdown of Piezo2 in DRG neurons selectively reduces fast-inactivating MA currents. (A) Representative images of colorimetric in situ hybridization for Piezo2 in Dorsal Root Ganglia (DRG) neurons using antisense (left panel) and sense (right panel) probes. (B) Representative traces of three typical MA inward currents expressed in DRG neurons are characterized by distinct inactivation kinetics. The neurons are subjected to a series of mechanical steps in 1 μm increments at a holding potential of −80 mV. Current inactivation is fitted with a bi-exponential equation giving fast time-constant (t) of 7.3 ms and slow time-constant>100 ms (left panel), or with a mono-exponential equation giving a time constant of 27 ms (middle panel). Some currents with t>30 ms are too slow to be efficiently fitted during the 150 ms lasting step stimulation (right panel). (C-D) Frequency histograms indicating the proportion of neurons transfected with scrambled siRNA (Ctr) or Piezo2 siRNA (siRNA) that respond to mechanical stimulation with MA currents characterized by their inactivation kinetic. Bars represent the mean±SEM of the proportion of neurons from seven separate experiments (B, n=12-19 neurons per condition and per experiment) or the proportion from all neurons pooled from all seven experiments (C); the numbers above bars in C represent the number of neurons. **, P<0.01. ns, not significantly different.

FIG. 11. Piezo2 mRNA is expressed in a subset of DRG neurons. Combined Piezo2 fluorometric in situ hybridization (left panels) with Peripherin (A, middle panel) and Neurofilament 200 (NF200) (B, middle planel) immunostaining in mouse DRG and (C) with TRPV1 (middle panel) immunostaining in rat DRG show that among Piezo2-positive neurons, 60% also expressed Peripherin (n=204 for Piezo2 and n=555 for Peripherin of which 23% express Piezo2, out of 1188 total neurons); 28%, NF200 (n=277 for Piezo2, and n=368 for NF200 of which 19% express Piezo2, out of 1203 total neurons), and 24%, TRPV1 (n=233 for Piezo2 and n=394 for TRPV1 of which 15% express Piezo2, out of 975 total neurons). Arrowheads show examples of neurons expressing Piezo2 and respective markers (A-C, right panels). Scale bars=50 μm.

FIG. 12. DRG and Piezo2 siRNA control experiments and comparison of MA current inactivation of DRG neurons. (A-B) siRNA-mediated knockdown of TRPA1 in cultured DRG neurons. (A) Representative traces of ratiometric calcium-imaging on cultured DRG neurons transfected with scrambled siRNA control (n=415 neurons, left panel) and TRPA1 siRNA (n=467 neurons, right panel). (B) Average percentage of neurons responding to mustard oil (MO, agonist of TRPA1 channels) and capsaicin (CAPS, agonist of TRPV1 channels) from 2 independent transfections were assayed 48 to 72 hours after transfection. While the percentage of responders to MO (100 μM) is significantly reduced upon siRNA treatment (scrambled: 15.34±3.69%; siRNA: 2.48±0.96%; P=0.0286, Mann Whitney test), responses to CAPS (0.5 μM) are unaffected by TRPA1 siRNA treatment (scrambled: 27.43±5.81%; siRNA: 32.28±3.84%; ns). Bars represent the mean±SEM. (C) Average maximal amplitude of Piezo2-induced MA currents in the presence or absence of Piezo2 siRNA. N2A cells transfected with Piezo1 siRNA to suppress endogenous MA currents were co-transfected with Piezo2 cDNA or Piezo2 cDNA+Piezo2 siRNA. (D) Histogram of time-constant of inactivation of MA currents recorded in scrambled siRNA transfected DRG neurons. Numbers of neurons expressing MA currents with time constant of inactivation ≦30 ms were plotted using a bin of 2.5 ms (the inactivation kinetic of currents with >30 ms time constant is too slow to be accurately fitted over 150 ms). Fit with double Gaussian equation shows two peaks centered at 7.2±0.5 ms and 16.0±2.1 ms, respectively. (E) Example trace of MA current showing comparison between the amount of current inactivated after 150 ms of mechanical stimulation (I inactivated, right double arrow) and the amount of current at the peak (I peak, left double arrow) (left panel). Percentage of DRG neurons with different degrees of current inactivation at 150 ms (I inactivated/I peak in 5% increments where 100% is completely inactivated) are compared between scrambled or Piezo2 siRNA transfected conditions (right panel, left and right bars in each pair, respectively).

### DETAILED DESCRIPTION OF THE INVENTION

### I. Introduction

The present invention identifies a role for multipass transmembrane proteins, called “Piezo” proteins, in mechanotransduction in cells. One or more Piezo proteins have been identified in animal, plant, and other eukaryotic species, including but not limited to, vertebrates (e.g., mammals such as humans and mice, birds such as chickens, and fish such as zebrafish), invertebrates (e.g., Ciona, Drosophila, Annopheles, and C. elegans), Arabidopsis, rice, and ciliates, although functional characterization of these Piezo proteins has not previously been reported. Piezo proteins have moderately conserved secondary structure and overall length, generally from about 2100 amino acids to about 4700 amino acids, with about 24-36 transmembrane domains located throughout the putative protein. It is demonstrated here for the first time that overexpression of Piezo induces robust mechanically-activated currents, while inhibition of Piezo expression reduces mechanically-activated currents. Without intending to limit the scope of the invention, it is believed that Piezos participate in mechanotransduction in cells as components of mechanically-activated cation channels.

Accordingly, the present invention provides methods of screening for agents that modulate the activity of mechanically-activated cation channel polypeptides by contacting the agents with polypeptides that are substantially identical to Piezo proteins. The present invention also provides antibodies against Piezo proteins that antagonize the activity of mechanically-activated cation channels and methods of ameliorating pain in a subject by administering said antibodies. The present invention further provides antisense oligonucleotides or siRNAs that inhibit the production of Piezo proteins and methods of ameliorating pain in a subject by administering said antisense oligonucleotides or siRNAs. The present invention further provides kits for practicing said methods.

### II. Assays for Modulators of Mechanically-Activated Cation Channel Activity

In one aspect, the present invention provides a method of screening for agents that modulate the activity of a mechanically-activated cation channel, the method comprising: contacting a mechanically-activated cation channel polypeptide with an agent; and selecting the agent that modulates the activity of the mechanically-activated cation channel polypeptide.

A. Expression of Mechanically-Activated Cation Channel Polypeptides

The mechanically-activated cation channel polypeptides, and the polynucleotides encoding said polypeptides, are substantially identical to members of the Piezo family of transmembrane proteins. In some embodiments, the mechanically-activated cation channel polypeptide is substantially identical to (e.g., at least 70%, 75%, 80%, 85%, 90%, 91%, 92%, 93%, 94% 95%, 96%, 97%, 98%, or 99% identical to) any one of SEQ ID NOs:2, 4, 18, or 20. In some embodiments, the mechanically-activated cation channel polypeptide comprises any of SEQ ID NOs:2, 4, 18, or 20.

In some embodiments, the method of screening for agents that modulate the activity of a mechanically-activated cation channel comprises contacting a cell comprising the mechanically-activated cation channel polypeptide that is substantially identical to (e.g., having at least 70% identity to) any one of SEQ ID NOs:2, 4, 18, or 20 with an agent, and selecting the agent that modulates the activity of the mechanically-activated cation channel polypeptide. In some embodiments, the cell endogenously expresses the mechanically-activated cation channel polypeptide. In some embodiments, the mechanically-activated cation channel polypeptide is heterologous to the cell.

Any cell that endogenously expresses a mechanically-activated cation channel polypeptide having at least 70% identity to any of SEQ ID NOs:2, 4, 18, or 20 at a detectable level may be used in the screening methods of the present invention. Whether a cell endogenously expresses the mechanically-activated cation channel polypeptide at a detectable level may be determined by any method of nucleic acid or protein expression known in the art. Nucleic acid may be detected using routine techniques such as Northern analysis, reverse-transcriptase polymerase chain reaction (RT-PCR), microarrays, sequence analysis, or any other methods based on hybridization to a nucleic acid sequence that is complementary to a portion of the marker coding sequence (e.g., slot blot hybridization). Protein may be detected using routine antibody-based techniques, for example, immunoassays such as ELISA, Western blotting, flow cytometry, immunofluorescence, and immunohistochemistry. Examples of cells that endogenously express mechanically-activated cation channel polypeptide having at least 70% identity to any one of SEQ ID NOs:2, 4, 18, or 20 at a detectable level include, but are not limited to, Neuro2A.

Alternatively, a mechanically-activated cation channel polypeptide can be heterologously expressed in a cell of interest using an expression cassette. An expression cassette, comprising a promoter operably linked to a polynucleotide encoding a mechanically-activated cation channel polypeptide as described herein, is generated using techniques that are known in the art.

In some embodiments, a polynucleotide encoding the mechanically-activated cation channel polypeptide is substantially identical to (e.g., at least 70%, 75%, 80%, 85%, 90%, 91%, 92%, 93%, 94% 95%, 96%, 97%, 98%, or 99% identical to) any one of SEQ ID NOs:1, 3, 17, or 19. In some embodiments, the polynucleotide encoding the mechanically-activated cation channel polypeptide comprises any one of SEQ ID NOs:1, 3, 17, or 19. The polynucleotides of the disclosure may be synthesized by chemical methods or prepared by techniques well known in the art. See, for example, Creighton, Proteins: Structures and Molecular Principles, W.H. Freeman & Co., New York, N.Y. (1983). Nucleotide sequences encoding the mechanically-activated cation channel polypeptides of the disclosure may be synthesized and/or cloned, and expressed according to techniques well known to those of ordinary skill in the art. See, for example, Sambrook, et al., Molecular Cloning, A Laboratory Manual, Vols. 1-3, Cold Spring Harbor Press, Cold Spring Harbor, N.Y. (1989).

The polynucleotide sequences encoding the mechanically-activated cation channels can be cloned from cDNA and genomic DNA libraries by hybridization with a probe, or isolated using amplification techniques with oligonucleotide primers. For example, mechanically-activated cation channel polynucleotides sequences can be isolated from mammalian nucleic acid (genomic or cDNA) libraries by hybridizing with a nucleic acid probe, the sequence of which can be derived from SEQ ID NOs:1, 3, 17, or 19. Methods for making and screening cDNA libraries are well known (see, e.g., Gubler & Hoffman, Gene 25:263-269 (1983); Sambrook et al., supra; Ausubel et al., supra). Suitable tissues from which mechanically-activated cation channel polypeptide RNA and cDNA can be isolated include, but are not limited to, dorsal root ganglia, nerve, neurons, bladder, colon, kidney, lung, and skin.

An alternative method of isolating mechanically-activated cation channel polynucleotides combines the use of synthetic oligonucleotide primers and amplification of an RNA or DNA template (see U.S. Pat. Nos. 4,683,195 and 4,683,202; PCR PROTOCOLS: A GUIDE TO METHODS AND APPLICATIONS (Innis et al., eds, 1990)). Methods such as polymerase chain reaction (PCR) and ligase chain reaction (LCR) can be used to amplify nucleic acid sequences of mechanically-activated cation channel directly from mRNA, from cDNA, from genomic libraries, or from cDNA libraries. Amplification techniques are known in the art, see, e.g., Sambrook, et al., 1989, Molecular Cloning, A Laboratory Manual, Vols. 1-3, Cold Spring Harbor Press, Cold Spring Harbor, N.Y. Primers can be prepared using the polynucleotide sequences that are available in publicly available databases. Genes amplified by the PCR reaction can be purified from agarose gels and cloned into an appropriate vector containing a selectable marker for propagation in a host. Such markers include but are not limited to dihydrofolate reductase or neomycin resistance for eukaryotic cell culture and tetracycline, ampicillin, or kanamycin resistance genes for culturing in E. coli and other bacteria.

To obtain high level expression of a cloned gene or nucleic acid, such as those cDNAs encoding the mechanically-activated cation channel, one typically subclones the mechanically-activated cation channel into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al. and Ausubel et al. Bacterial expression systems for expressing a mechanically-activated cation channel polypeptide are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available.

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the mechanically-activated cation channel encoding nucleic acid in host cells. A typical expression cassette thus contains a promoter operably linked to the nucleic acid sequence encoding the mechanically-activated cation channel and signals required for efficient polyadenylation of the transcript, ribosome binding sites, and translation termination. The nucleic acid sequence encoding the mechanically-activated cation channel may typically be linked to a cleavable signal peptide sequence to promote secretion of the encoded protein by the transformed cell. Such signal peptides would include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette can also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as—thymidine kinase, hygromycin B phosphotransferase, and dihydrofolate reductase. Alternatively, high yield expression systems not involving gene amplification are also suitable, such as using a baculovirus vector in insect cells, with a mechanically-activated cation channel encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary.

Recombinant expression vectors comprising a mechanically-activated cation channel coding sequence driven by a heterologous promoter may be introduced into the genome of the desired host cell using any of a variety of well known procedures. These procedures include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the mechanically-activated cation channel.

B. Screening for Modulation of Cation Channel Activity

1. Cation Channel Activity Assays

Piezos are components of mechanically-activated cation channels. The activity of mechanically-activated cation channels comprising polypeptides that are substantially identical to Piezos can be assessed using a variety of in vitro and in vivo assays, e.g., measuring electrophysiological changes such as changes in current (both in mechanically-sensitive assays and assays independent of mechanical stimulation), measuring second messengers and transcription levels, measuring ligand binding, measuring cation influx, and using voltage-sensitive dyes, ion-sensitive dyes (e.g., Ca²⁺), and the like. Furthermore, such assays can be used to test for inhibitors and activators of mechanically-activated cation channels. Such modulators are useful for treating various disorders involving mechanically-activated cation channels.

In some embodiments, agents that modulate (activate or inhibit) the activity of the mechanically-activated cation channel are identified in an initial screen using an assay that measures an aspect independent of mechanical stimulation, e.g., voltage-clamp or patch-clamp assay, voltage-sensitive dye, ion-sensitive dye, cation influx assay, etc. Agents that are identified as agonizing or inhibiting the activity of the mechanically-activated cation channel using such an assay can then be screened for the ability to modulate the activity of the mechanically-activated cation channel in a mechanically-dependent manner by testing the agonistic or antagonistic effects of the agent in a mechanically-sensitive assay, e.g, using a piezoelectrically-driven pressure assay or membrane stretch assay.

In some embodiments, agents that modulate the activity of the mechanically-activated cation channel are identified in an initial screen using a mechanically-sensitive assay as described herein.

Modulators are tested using a biologically active mechanically-activated cation channel polypeptide that is substantially identical to Piezo, either recombinant or naturally occurring. The mechanically-activated cation channel polypeptide can be isolated, co-expressed or expressed in a cell, or expressed in a membrane derived from a cell. Modulation is tested using one of the in vitro or in vivo assays described above. Samples or assays that are treated with a potential mechanically-activated cation channel inhibitor or activator are compared to control samples without the test compound, to examine the extent of modulation. Control samples (untreated with activators or inhibitors) are assigned a relative mechanically-activated cation channel activity value of 100. Inhibition of the mechanically-activated cation channel is achieved when the mechanically-activated cation channel activity value relative to the control is about less than 90%, e.g., less than 75%, less than 50%, or less than 25%. Activation of the mechanically-activated cation channel is achieved when the mechanically-activated cation channel activity value relative to the control is more than 110%, more than 125%, more than 150%, or more than 200% higher. Compounds that increase the flux of ions will cause a detectable increase in the ion current density by increasing the probability of a mechanically-activated cation channel being open, by decreasing the probability of it being closed, by increasing conductance through the channel, and/or by allowing the passage of ions.

Changes in ion flux may be assessed by determining changes in polarization (i.e., electrical potential) of the cell or membrane expressing the mechanically-activated cation channel. A method to determine changes in cellular polarization is by measuring changes in current (thereby measuring changes in polarization) with voltage-clamp and patch-clamp techniques, e.g., the “cell-attached” mode, the “inside-out” mode, and the “whole cell” mode (see, e.g., Ackerman et al., New Engl. J. Med. 336:1575-1595 (1997)). Whole cell currents are conveniently determined using the standard methodology (see, e.g., Hamill et al., Pflugers. Archiv. 391:85-100 (1981)). Other known assays include: radiolabeled ion flux assays and fluorescence assays using voltage-sensitive or ion-sensitive dyes (see, e.g., Vestergarrd-Bogind et al., J. Membrane Biol. 88:67-75 (1988); Daniel et al., J. Pharmacol. Meth. 25:185-193 (1991); Holevinsky et al., J. Membrane Biology 137:59-70 (1994)). Assays for compounds capable of inhibiting or increasing cation flux through the mechanically-activated cation channels can be performed by application of the compounds to a bath solution in contact with and comprising cells having a channel of the present invention (see, e.g., Blatz et al., Nature 323:718-720 (1986); Park, J. Physiol. 481:555-570 (1994)). Generally, the compounds to be tested are present in the range from 1 pM to 100 mM.

The effects of the test compounds upon the function of the channels can be measured by changes in the electrical currents or ionic flux or by the consequences of changes in currents and flux. Changes in electrical current or ionic flux are measured by either increases or decreases in flux of ions such as cations (e.g., calcium, sodium, potassium, or magnesium ions). The ions can be measured in a variety of standard ways. They can be measured directly by concentration changes of the ions, e.g., changes in intracellular concentrations, or indirectly by membrane potential or by radio-labeling of the ions. Consequences of the test compound on ion flux can be quite varied. Accordingly, any suitable physiological change can be used to assess the influence of a test compound on the channels of this invention.

In some embodiments, the mechanically-activated cation channel polypeptide that is used in the assay will have the sequence displayed in the following GenBank accession numbers: human Piezo1—NP_{—}001136336.2; mouse Piezo1—NP_{—}001032375.1; chicken Piezo1—XP_{—}414209.2 or XP_{—}423106.2; zebrafish Piezo1—XP_{—}696355.4; human Piezo2—NP_{—}071351.2; mouse Piezo2—NP_{—}001034574.3; chicken Piezo2—XP_{—}419138.2; zebrafish Piezo2—XP_{—}00266625.1; or a conservatively modified variant, ortholog, and/or substantially identical variant thereof. Generally, the amino acid sequence identity will be at least 65%, e.g., at least 70%, 75%, 80%, 85%, 90%, 95%, 96%, 97%, 98%, or at least 99%.

Piezo orthologs, alleles, polymorphic variants, and conservatively modified variants will generally confer substantially similar properties on a mechanically-activated cation channel as described above. In some embodiments, the cell placed in contact with a compound that is suspected to be a Piezo homolog is assayed for increasing or decreasing ion flux in a eukaryotic cell, e.g., an oocyte of Xenopus (e.g., Xenopus laevis) or a mammalian cell such as a CHO or HeLa cell or as assayed in binding studies using similar cell types. Channels that are affected by compounds in ways similar to Piezo are considered homologs or orthologs of Piezo.

2. Mechanically-Sensitive Cation Channel Activity Assays

In some embodiments, agents are screened for the ability to modulate mechanically-activated electrophysiological changes in a channel comprising a polypeptide that is substantially identical to Piezo. Mechanically-sensitive assays are known in the art and include, for example, piezo-driven pressure, patch membrane stretch, shear stress, osmotic challenges, and amphipathic compounds.

As a non-limiting example, the ability of an agent to modulate mechanically-activated electrophysiological changes in a cell can be assayed using whole cell recordings measuring stimulation by a piezo-electrically driven mechanical probe. Methods of assaying piezo-driven pressure have been described, see, e.g., Hu and Lewin, J. Physiol. 577:815-828 (2006)). Briefly, a fire-polished glass probe is typically positioned close to the cell surface at an angle ≧45°. The probe is driven toward the cell at a controlled velocity and for a controlled length of time using a Clampex (Molecular Devices, Sunnyvale, Calif.)-controlled piezo-electric crystal microstage and mechanically-activated inward current is recorded. One of skill in the art will recognize that it may be useful to vary parameters such as voltage and the intensity, velocity, and duration of the mechanical stimulus.

As another non-limiting example, the ability of an agent to modulate mechanically-activated electrophysiological changes in a cell can be assayed by stretch of the plasma membrane through a patch pipette in cell-attached mode. Methods of assaying patch membrane stretch have been described, see, e.g., Gil et al., Proc. Natl. Acad. Sci. USA 96:14594-14599 (1999)). Briefly, on-cell, or cell-attached, patches are formed by pressing the tip of a heat-polished patch pipette against the membrane of the cell and then applying slight negative pressure to the patch pipette using a Clampex-controlled pressure clamp (e.g., HSPC-1 pressure clamp, ALA Scientific Instruments, Westbury, N.Y.) and mechanically-activated inward current is recorded. One of skill in the art will recognize that it may be useful to vary parameters such as voltage and the amount and duration of pressure applied.

3. Solid State and High Throughput Assays

In the high throughput assays of the invention, it is possible to screen up to several thousand, ten thousand, hundred thousand or more different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100- about 1500 different compounds. It is possible to assay several different plates per day; assay screens for up to about 6,000-20,000 or more different compounds is possible using the integrated systems of the invention. More recently, microfluidic approaches to reagent manipulation have been developed, e.g., by Caliper Technologies (Palo Alto, Calif.), and can be used.

As a non-limiting example, potential modulators can be screened for effect on mechanically-activated cation channels using a high throughput electrophysiological screening system such as IonWorks™ HT (Molecular Devices, Sunnyvale, Calif.). Briefly, the IonWorks™ HT system measures whole-cell current from multiple cells simultaneously using a 384-well plate. Cells expressing a voltage-gated ion channel of interest are dispensed into individual wells in parallel with an onboard fluidics system and a single cell is subsequently positioned over a single small aperture within each well, the aperture separating two isolated fluid-filled upper and lower chambers, each containing buffered solutions and separate electrodes. The positioned cells form stable seals over the apertures, impeding electrical flow between the two chambers. A cell membrane pore-forming agent (e.g., amphotericin B) is introduced into the lower chamber, creating an electrical pathway through the portion of the cell membrane exposed through the small aperture in each of the wells. An electronics head containing 48 electrodes is positioned into the upper chamber clamping the cell membrane potential and subsequently recording ionic currents from 48 cells in parallel. Compounds are aspirated from 96- or -384 well microplates and dispensed in parallel with a 12-channel fluidics head pipettor.

The molecule of interest can be bound to the solid state component, directly or indirectly, via covalent or non covalent linkage, e.g., via a tag. The tag can be any of a variety of components. In general, a molecule which binds the tag (a tag binder) is fixed to a solid support, and the tagged molecule of interest (e.g., a mechanically-activated cation channel polypeptide) is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly-Gly sequences of between about 5 and 200 amino acids. Such flexible linkers are known to persons of skill in the art. For example, poly(ethylene glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

4. Validation

Agents that are initially identified by any of the foregoing screening methods can be further tested to validate the apparent activity and/or determine other biological effects of the agent. In some cases, the identified agent is administered to an animal (e.g., a non-human mammal such as a mouse) to determine the effect of the agent on pain sensitivity.

C. Agents that Modulate Cation Channel Activity

The compounds tested as modulators of the mechanically-activated cation channels can be any small chemical compound, or a biological entity, such as a protein, sugar, nucleic acid or lipid. Typically, test compounds will be small chemical molecules and peptides. Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al, J. Amer. Chem. Soc. 114:6568 (1992)), nonpeptidal peptidomimetics with glucose scaffolding (Hirschrnann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

### III. Inhibiting Expression of Mechanically-Activated Cation Channels

A. Antibodies

In another aspect, the present invention provides antibodies that specifically bind to the mechanically-activated cation channels. Such antibodies are useful, e.g., for ameliorating or treating pain or itch in a subject. Suitable antibodies include, but are not limited to, monoclonal antibodies, humanized antibodies, chimeric antibodies, and antibody fragments (i.e., Fv, Fab, (Fab′)₂, or scFv).

In some embodiments, the antibody selectively binds to a mechanically-activated cation channel polypeptide having at least 70% amino acid sequence identity to SEQ ID NO:2, SEQ ID NO:4, SEQ ID NO:18, or SEQ ID NO:20. In some embodiments, the antibody selectively binds to a polypeptide comprising SEQ ID NO:2, SEQ ID NO:4, SEQ ID NO:18, or SEQ ID NO:20.

Monoclonal antibodies are obtained by various techniques familiar to those skilled in the art. Briefly, spleen cells from an animal immunized with a desired antigen are immortalized, commonly by fusion with a myeloma cell (see, for example, Kohler & Milstein, Eur. J. Immunol. 6: 511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse et al., Science 246: 1275-1281 (1989).

Monoclonal antibodies are collected and titered against the immunogen in an immunoassay, for example, a solid phase immunoassay with the immunogen immobilized on a solid support. Monoclonal antibodies will usually bind with a K_{d} of at least about 0.1 mM, more usually at least about 1 μM, and can often be designed to bind with a K_{d} of 1 nM or less.

In an exemplary embodiment, an animal, such as a rabbit or mouse is immunized with a mechanically-activated cation channel polypeptide, or an nucleic acid construct encoding such a polypeptide. The antibodies produced as a result of the immunization can be isolated using standard methods.

The immunoglobulins, including binding fragments and other derivatives thereof, of the present invention may be produced readily by a variety of recombinant DNA techniques, including by expression in transfected cells (e.g., immortalized eukaryotic cells, such as myeloma or hybridoma cells) or in mice, rats, rabbits, or other vertebrate capable of producing antibodies by well known methods. Suitable source cells for the DNA sequences and host cells for immunoglobulin expression and secretion can be obtained from a number of sources, such as the American Type Culture Collection (Catalogue of Cell Lines and Hybridomas, Fifth edition (1985) Rockville, Md.).

In some embodiments, the antibody is a humanized antibody, i.e., an antibody that retains the reactivity of a non-human antibody while being less immunogenic in humans. This can be achieved, for instance, by retaining the non-human CDR regions that are specific for mechanically-activated cation channel, and replacing the remaining parts of the antibody with their human counterparts. See, e.g., Morrison et al., PNAS USA, 81:6851-6855 (1984); Morrison and Oi, Adv. Immunol., 44:65-92 (1988); Verhoeyen et al., Science, 239:1534-1536 (1988); Padlan, Molec. Immun., 28:489-498 (1991); Padlan, Molec. Immun., 31(3):169-217 (1994). Techniques for humanizing antibodies are well known in the art and are described in e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534. Humanized antibodies are further described in, e.g., Winter and Milstein (1991) Nature 349:293. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells. The CDRs for producing the immunoglobulins of the present invention will be similarly derived from monoclonal antibodies capable of specifically binding to a mechanically-activated cation channel.

In some cases, transfer of a CDR to a human framework leads to a loss of specificity for the humanized antibody. In these cases, back mutation can be introduced into the framework regions of the human portion of the antibody. Methods of making back mutations are well known in the art and are described in, e.g., Co et al., PNAS USA 88; 2269-2273 (1991) and WO 90/07861.

The mechanically-activated cation channel—specific antibody can also be chimeric, so that all or most of the variable region is retained, but the constant region replaced. For example, a murine variable region that possesses mechanically-activated cation channel binding activity may be combined with human constant regions, or constant regions from another mammal for use in veterinary treatments.

In some embodiments, the antibodies are antibody fragments such as Fab, F(ab′)₂, Fv or scFv. The antibody fragments can be generated using any means known in the art including, chemical digestion (e.g., papain or pepsin) and recombinant methods. Methods for isolating and preparing recombinant nucleic acids are known to those skilled in the art (see, Sambrook et al., Molecular Cloning. A Laboratory Manual (2d ed. 1989); Ausubel et al., Current Protocols in Molecular Biology (1995)). The antibodies can be expressed in a variety of host cells, including E. coli, other bacterial hosts, yeast, and various higher eukaryotic cells such as the COS, CHO, and HeLa cells lines and myeloma cell lines.

B. siRNA and Antisense Oligonucleotides

In another aspect, the present invention provides oligonucleotide and polynucleotide sequences that inhibit production of a mechanically-activated cation channel polypeptide. Such inhibitory nucleic acid sequences are useful, e.g., for ameliorating or treating pain or itch in a subject. Suitable oligonucleotides and polynucleotides include, but are not limited to, siRNA and antisense oligonucleotides.

In some embodiments, the oligonucleotide or polynucleotide is complementary to at least 15 contiguous nucleotides of a polynucleotide that is at least 70% identical to SEQ ID NOs:1, 3, 17, or 19. In some embodiments, the oligonucleotide or polynucleotide is complementary to at least 18, at least 20, at least 22, at least 25, at least 30, at least 40, or at least 50 contiguous nucleotides of a polynucleotide that is at least 70% identical to SEQ ID NOs:1, 3, 17, or 19. In some embodiments, the oligonucleotide or polynucleotide comprises any of SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, or SEQ ID NO:16.

1. siRNA

Double stranded siRNA that corresponds to a gene encoding a mechanically-activated cation channel polypeptide can be used to silence the transcription and/or translation of the mechanically-activated cation channel polypeptide by inducing degradation of mRNA transcripts, and thus ameliorate or treat pain or itch by preventing expression of the mechanically-activated cation channel polypeptide. The siRNA is typically about 5 to about 100 nucleotides in length, more typically about 10 to about 50 nucleotides in length, most typically about 15 to about 30 nucleotides in length. siRNA molecules and methods of generating them are described in, e.g., Bass, 2001, Nature, 411, 428-429; Elbashir et al., 2001, Nature, 411, 494-498; WO 00/44895; WO 01/36646; WO 99/32619; WO 00/01846; WO 01/29058; WO 99/07409; and WO 00/44914. A DNA molecule that transcribes dsRNA or siRNA (for instance, as a hairpin duplex) also provides RNAi. DNA molecules for transcribing dsRNA are disclosed in U.S. Pat. No. 6,573,099, and in U.S. Patent Application Publication Nos. 2002/0160393 and 2003/0027783, and Tusch1 and Borkhardt, Molecular Interventions, 2:158 (2002). For example, dsRNA oligonucleotides that specifically hybridize to the nucleic acid sequences set forth in SEQ ID NO:1, SEQ ID NO:3, SEQ ID NO:17, or SEQ ID NO:19 can be used in the methods of the present invention. A decrease in the severity of pain symptoms in comparison to symptoms detected in the absence of the interfering RNA can be used to monitor the efficacy of the siRNA.

siRNA can be delivered to the subject using any means known in the art, including by injection, inhalation, or oral ingestion of the siRNA. Another suitable delivery system for siRNA is a colloidal dispersion system such as, for example, macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. Nucleic acids, including RNA and DNA within liposomes and be delivered to cells in a biologically active form (Fraley, et al., Trends Biochem. Sci., 6:77, 1981). Liposomes can be targeted to specific cell types or tissues using any means known in the art.

2. Antisense Oligonucleotides

Antisense oligonucleotides that specifically hybridize to nucleic acid sequences encoding mechanically-activated cation channel polypeptides can also be used to silence the transcription and/or translation of the mechanically-activated cation channel polypeptide, and thus ameliorate or treat pain or itch. For example, antisense oligonucleotides that specifically hybridize to the nucleic acid sequences set forth in SEQ ID NO:1, SEQ ID NO:3, SEQ ID NO:17, or SEQ ID NO:19 can be used in the methods of the present invention. A decrease in the severity of pain symptoms in comparison to symptoms detected in the absence of the antisense nucleic acids can be used to monitor the efficacy of the antisense nucleic acids.

Antisense nucleic acids are DNA or RNA molecules that are complementary to at least a portion of a specific mRNA molecule (see, e.g., Weintraub, Scientific American, 262:40 (1990)). Typically, synthetic antisense oligonucleotides are generally between 15 and 25 bases in length. Antisense nucleic acids may comprise naturally occurring nucleotides or modified nucleotides such as, e.g., phosphorothioate, methylphosphonate, and -anomeric sugar-phosphate, backbone-modified nucleotides.

In the cell, the antisense nucleic acids hybridize to the corresponding mRNA, forming a double-stranded molecule. The antisense nucleic acids interfere with the translation of the mRNA, since the cell will not translate a mRNA that is double-stranded. Antisense oligomers of about 15 nucleotides are preferred, since they are easily synthesized and are less likely to cause problems than larger molecules when introduced into the target nucleotide mutant producing cell. The use of antisense methods to inhibit the in vitro translation of genes is well known in the art (Marcus-Sakura, Anal. Biochem., 172:289, (1988)). Less commonly, antisense molecules which bind directly to the DNA may be used.

Delivery of antisense polynucleotides specific for a gene encoding a mechanically-activated cation channel can be achieved using any means known in the art including, e.g., direct injection, inhalation, or ingestion of the polynucleotides. In addition, antisense polynucleotides can be delivered using a recombinant expression vector (e.g., a viral vector based on an adenovirus, a herpes virus, a vaccinia virus, or a retrovirus) or a colloidal dispersion system (e.g., liposomes) as described herein.

### IV. Methods of Treating Mechanically-Activated Cation Channel-Mediated Diseases

In yet another aspect, the present invention provides compositions comprising antagonists of mechanically-activated cation channels. The compositions of the invention can be provided to ameliorate or treat diseases or conditions which involve pain transmitted via mechanically-activated cation channels.

Mechanically-activated cation channels are implicated in the transmission of various sensations such as touch, pressure, vibration, proprioception, and pain. Accordingly, antagonists of mechanically-activated cation channels may be administered to a subject having a disease or condition characterized by alterations in the transmission of these sensations, e.g., alterations in touch or pain pathways that result in acute or chronic pain, heightened sensitivity to pain or touch, or heightened intensity of pain or touch.

In one embodiment, the compositions of the invention (e.g., the antibodies that selectively bind to mechanically-activated cation channels or the oligonucleotides or polynucleotides that inhibit production of a mechanically-activated cation channel polypeptide) can be provided to a subject having pain selected from the group consisting of acute mechanical pain, chronic mechanical pain, mechanical hyperalgesia, mechanical allodynia, arthritis, inflammation, dental pain, cancer pain, and labor pain.

The compositions of the invention can be administered in a single dose, multiple doses, or on a regular basis (e.g., daily) for a period of time (e.g., 2, 3, 4, 5, 6, days or 1-3 weeks or more).

The compositions of the invention can be administered directly to the mammalian subject to block mechanically-activated cation channel activity using any route known in the art, including e.g., by injection (e.g., intravenous, intraperitoneal, subcutaneous, intramuscular, or intrademal), inhalation, transdermal application, rectal administration, or oral administration.

The pharmaceutical compositions of the invention may comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 17th ed., 1989).

The compositions of the invention, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, orally, nasally, topically, intravenously, intraperitoneally, or intrathecally. The formulations of compounds can be presented in unit-dose or multi-dose sealed containers, such as ampoules and vials. Solutions and suspensions can be prepared from sterile powders, granules, and tablets of the kind previously described. The modulators can also be administered as part a of prepared food or drug.

Formulations suitable for oral administration can comprise: (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The dose administered to a patient, in the context of the present invention should be sufficient to effect a beneficial response in the subject over time, e.g., a reduction in pulmonary capillary hydrostatic pressure, a reduction in fluid in the lungs, a reduction in the rate of fluid accumulation in the lungs, or a combination thereof. The optimal dose level for any patient will depend on a variety of factors including the efficacy of the specific modulator employed, the age, body weight, physical activity, and diet of the patient, on a possible combination with other drugs, and on the severity of the pain. The size of the dose also will be determined by the existence, nature, and extent of any adverse side-effects that accompany the administration of a particular compound or vector in a particular subject.

In determining the effective amount of the antagonists of mechanically-activated cation channels to be administered a physician may evaluate circulating plasma levels of the antagonist and antagonist toxicity. In general, the dose equivalent of an antagonist is from about 1 ng/kg to 10 mg/kg for a typical subject.

### V. Kits

The present invention also provides for kits for screening for modulators of mechanically-activated cation channels and for treating pain in a subject. Such kits can be prepared from readily available materials and reagents. For example, a kit for screening for modulators of mechanically-activated cation channels can comprise any one or more of the following materials: a mechanically-activated cation channel polypeptide, reaction tubes, and instructions for testing mechanically-activated cation channel activity. A kit for treating pain in a subject can comprise any one or more of the following materials: an antibody or inhibitory oligonucleotide or polynucleotide composition as described herein and instructions for administering the composition to a subject. A wide variety of kits and components can be prepared according to the present invention, depending upon the intended user of the kit and the particular needs of the user.

### EXAMPLES

The following examples are offered to illustrate, but not to limit the claimed invention.

### Example 1

### Materials and Methods

This example provides a description of particular materials and methods used in the following examples. One of skill in the art would readily understand that various modifications of substitutions in the described methods may be used.

Cell culture and transient transfection. Neuro2A cells were grown in Eagle's Minimum Essential Medium containing 4.5 mg.ml⁻¹ glucose, 10% fetal bovine serum, 50 units.ml⁻¹ penicillin and 50 μg.ml⁻¹ streptomycin. C2C12 or Human Embryonic Kidney 293T (HEK293T) cells were grown in Dulbecco's Modified Eagle Medium containing 4.5 mg.ml⁻¹ glucose, 10% fetal bovine serum, 50 units.ml⁻¹ penicillin and 50 μg.ml⁻¹ streptomycin. Cells were plated onto 35 mm dishes or 12-mm round glass coverslips placed in 24-well plates and transfected using lipofectamine 2000 (Invitrogen) according to the manufacturer's instructions. For Piezo1 overexpression experiment, 500 to 1000 ng.ml⁻¹ of Piezo1-IRES-GFP or vector only were transfected, and cells were recorded 12-48 hours later. For Piezo2 overexpression experiments, 600 to 1000 ng.ml⁻¹ of mPiezo2 or vector were co-transfected with 300 ng.ml⁻¹ GFP to identify transfected cells and cells were recorded 12-48 hours later.

For siRNA experiment, 20 nM total siRNA and 300 or 500 ng.ml⁻¹ GFP to identify transfected cells were co-transfected and cells were used 3 days after transfection. siRNA of Smartpool I directed against mPiezo1 were purchased from Qiagen (Target sequences: CACCGGCATCTACGTCAAATA (siRNA1) (SEQ ID NO:5), ACCAAGAAATACAACCATCTA (siRNA2) (SEQ ID NO:6), TCGGCGCTTGCTAGAACTTCA (siRNA3) (SEQ ID NO:7), and CGGAATCCTGCTGCTGCTATA (siRNA4) (SEQ ID NO:8)) and used at 5 nM each together in Smartpool I or at 20 nM separately. siRNA4 was toxic at 20 nM, as it caused cell detachment and subsequent death 3 days after transfection. Smartpool II siRNA was a pool of 4 different siRNA purchased from Dharmacon (Target sequences: GAAAGAGATGTCACCGCTA (SEQ ID NO:9), GCATCAACTTCCATCGCCA (SEQ ID NO:10), AAAGACAGATGAAGCGCAT (SEQ ID NO:11), GGCAGGATGCAGTGAGCGA (SEQ ID NO:12)). For the Piezo2 siRNA experiment in N2A cells, 600 ng.ml⁻¹ of mPiezo2 and 300 ng.ml⁻¹ GFP were co-transfected with 20 nM Piezo1 siRNA1 only or together with 20 nM Piezo2 siRNA. Cells were recorded 3 days after transfection. siRNA directed against mPiezo2 was a pool of 4 different siRNA purchased from Dharmacon (Target sequences: GAATGTAATTGGACAGCGA (SEQ ID NO:13), TCATGAAGGTGCTGGGTAA (SEQ ID NO:14), GATTATCCATGGAGATTTA (SEQ ID NO:15), GAAGAAAGGCATGAGGTAA (SEQ ID NO:16)).

DRG Culture and siRNA.

Preparation and culture of mouse dorsal root ganglion neurons (from male C57B16 mice) were performed as described previously (M. Chalfie, Nat Rev Mol Cell Biol 10, 44 (January, 2009)) with the following modifications: Growth medium was supplemented with 100 ng/ml nerve growth factor (NGF), 50 ng/ml GDNF, 50 ng/ml BDNF, 50 ng/ml NT-3, 50 ng/ml NT-4. Small interference RNA (siRNA)-mediated knockdown was achieved by nucleofection of siRNA into freshly isolated DRG neurons using the SCN nucleofector kit with the nucleofector device according to the manufacturer's instructions (SCN Basic Neuro program 6; Lonza AG). DRG neurons isolated from one mouse were used per siRNA tested. siRNAs were used at 150 nM-250 nM for TRPA1 (smartpool, Qiagen) and 250 nM for Piezo2 (smartpool, Qiagen), concentrations of scrambled controls (Qiagen) were adjusted accordingly. After nucleofection, neurons were allowed to recover in RPMI medium for 10 min at 37° C., growth medium (without antibiotics and without AraC) was added and neurons were plated on poly-D-lysine coated coverslips, previously coated with laminin (2 μg.ml⁻¹). 2-4 hrs after transfection, half of the growth medium was exchanged with fresh medium, and neurons were grown for 48-72 hours.

Electrophysiology.

Patch-clamp experiments were performed in standard whole-cell or cell attached recordings using an Axopatch 200B amplifier (Axon Instruments). Patch pipettes had resistance of 2-3 MΩ when filled with an internal solution consisting of (in mM) 133 CsCl, 10 HEPES, 5 EGTA, 1 CaCl₂, 1 MgCl₂, 4 MgATP, and 0.4 Na₂GTP (pH adjusted to 7.3 with CsOH). The extracellular solution consisted of (in mM) 127-130 NaCl, 3 KCl, 1 MgCl₂, 10 HEPES, 2.5 CaCl₂, 10 glucose (pH adjusted to 7.3 with NaOH). NMDG solution consisted of (in mM) 150 NMDG, 10 HEPES (pH 7.5). For ion selectivity experiments, internal solution consisted of (in mM) 150 CsCl, 10 Hepes (pH 7.3 with CsOH), monovalent external solutions consisted of (in mM) 150 NaCl or KCl, 10 HEPES (pH 7.3 with NaOH or KOH) and divalent external solutions consisted of (in mM) 100 CaCl₂ or MgCl₂, 10 HEPES (pH 7.3 with CsOH). For cell attached recordings, pipette were filled with a solution consisting of (in mM) 130 NaCl, 5 KCl, 10 HEPES, 1 CaCl₂, 1 MgCl₂, 10 TEA-C1 (pH 7.3 with NaOH) and external solution used to zero the membrane potential consisted of (in mM) 140 KCl, 10 HEPES, 1 MgCl₂, 10 glucose (pH 7.3 with KOH). All experiments were done at room temperature. Currents were sampled at 20 kHz and filtered at 2 kHz. Voltages were not corrected for a liquid junction potential excepted for ion selectivity experiments were LJP were calculated using Clampex 10.1 software (Axon Instruments). Leak currents before mechanical stimulations were subtracted off-line from the current traces. 10 mM ruthenium red stock solution was prepared in DMSO; 100 mM gadolinium stock solution, in water.

Mechanical Stimulation.

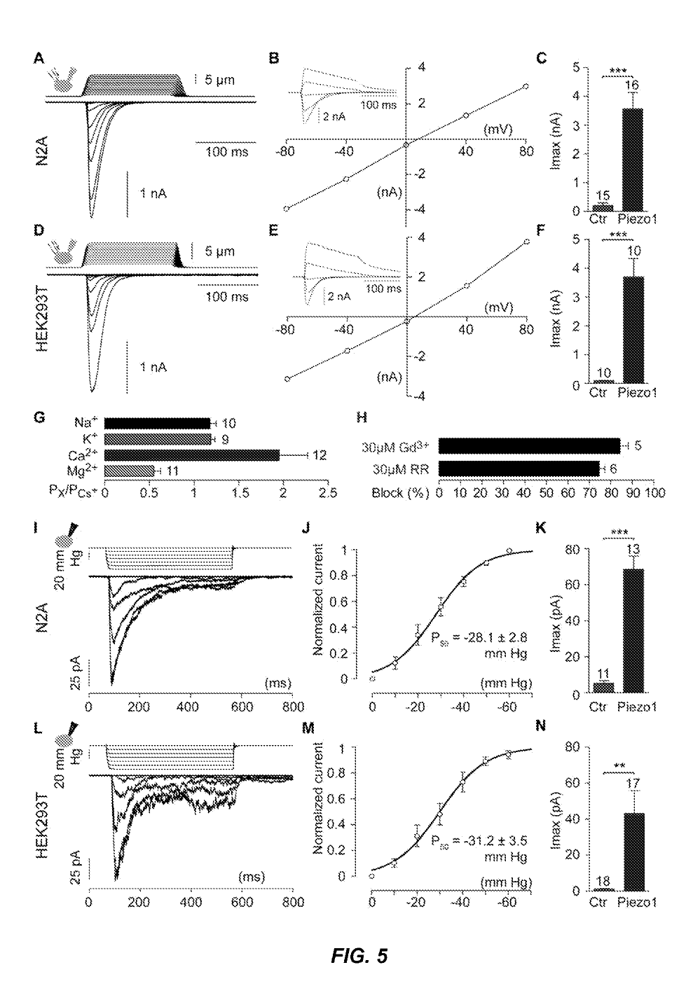

For whole-cell recordings mechanical stimulation was achieved using a fire-polished glass pipette (tip diameter 3-4 μm) positioned at an angle of 80° to the cell being recorded. Downward movement of the probe toward the cell was driven by a Clampex controlled piezo-electric crystal microstage (E625 LVPZT Controller/Amplifier; Physik Instrumente). The probe was typically positioned approximately 2 μm from the cell body. The piezoelectrically driven stimulus intensity used to measure the threshold of MA current activation was defined as the distance traveled beyond that which touched the cell. The probe had a velocity of 1 μm/ms during the ramp segment of the command for forward motion and the stimulus was applied for 150 ms. To assess the mechanical sensitivity of a cell, a series of mechanical steps in 1 μm increments were applied every 10 s, which allowed full recovery of mechanosensitive currents. Inward MA currents were recorded at a holding potential of −80 mV. For I-V relationship recordings, voltage steps were applied 0.7 s before the mechanical stimulation from a holding potential of −60 mV. For recordings of MA currents in DRG neurons, the inactivation kinetics at a holding potential of −80 mV of traces of currents reaching at least 75% of the maximal amplitude of current elicited per cell were fitted with mono-exponential equation (or in some case bi-exponential equation for the rapidly-adapting currents, accordingly to previous reports (O. P. Hamill, B. Martinac, Physiol Rev 81, 685 (April, 2001)) and using the fast time constant for analysis, see FIG. 5A left panel) giving a value of τ_{inac} per responsive neuron that was used for analysis.

For cell-attached recordings, membrane patches were stimulated with brief negative pressure pulses through the recording electrode using a Clampex controlled pressure clamp HSPC-1 device (ALA-scientific). Otherwise stated, stretch-activated channels were recorded at a holding potential of −80 mV with pressure steps from 0 to −60 mm Hg (−10 mm Hg increments), and 3-10 recording traces were averaged per cell for analysis. Current-pressure relationships were fitted with a Boltzmann equation of the form: I(P)=[1+exp(−(P−P₅₀)/s)]⁻¹, where I is the peak of stretch-activated current at a given pressure, P is the applied patch pressure (in mm Hg), P₅₀ is the pressure value that evoked a current value which is 50% of Imax, and s reflects the current sensitivity to pressure.

Determination of permeability ratios. Reversal potentials for each cell in each solution were determined by interpolation of the respective current-voltage data. In these experiments, I-V relationships were performed from a holding potential of −60 mV with voltage steps ranging from −60 mV to +60 mV (before liquid junction potential correction) in 20 mV increments.

The ratio of permeabilities, P_{X}/P_{Cs}, was determined for each test cation X for each cell from the reversal potential of the MA activated whole-cell current when that cation was the major external cation. The Goldman-Hodgkin-Katz (GHK) equation (G. B. Monshausen, S. Gilroy, Trends Cell Biol 19, 228 (May, 2009)), simplified for a single permeant cation on each side of the membrane, was employed:

$$\text{?} = \frac{HT}{z \text{?}} ln \frac{{F}_{X} \text{?}}{\text{?} \text{?}}$$

$$\text{?} \text{indicates text missing or illegible when filed}$$

where RT/zF has the value of 25.5 at 23° C. For the divalent cations the appropriately modified equation was used:

$$\text{?} = \frac{RT}{\text{?}} ln ( \sqrt{\frac{4 \text{?} \text{?}}{\text{?} \text{?}} + \frac{1}{4}} - \frac{1}{2} )$$

$$\text{?} \text{indicates text missing or illegible when filed}$$

Ratio P_{X}/P_{Cs} were presented for each cation as mean±SEM.

Ratiometric calcium imaging. Intracellular Ca²⁺ imaging experiments were performed by washing cells three times with Ca²⁺ imaging buffer [1×Hanks Balanced Salt Solution (HBSS, 1.3 mM Ca²⁺) supplemented with 10 mM HEPES], then loaded with ratiometric Ca²⁺ indicator dye Fura-2/AM (Molecular Probes) for 30 minutes at room temperature, according to the manufacturer's recommendations. Cells were washed three times prior to imaging on an inverted microscope. Fura-2 fluorescence was measured by illuminating the cells with an alternating 340/380 nm light. Fluorescence intensity was measured at 510 nm. The intracellular Ca²⁺ concentration is expressed as the 340/380 ratio.

Ratiometric calcium imaging of cultured DRG neurons was performed essentially as described [1]. Experiments were conducted at 37° C. 48-72 hrs after plating. Threshold for activation was set at 40% above the averaged baseline from 5 time points immediately before addition of MO (100 μM). Capsaicin (CAPS, 0.5 μM) was added at the end of each experiment to control for siRNA specificity, neuronal health and responsiveness. All experimental groups to be compared were processed in parallel using the same DRG culture preparation (2 independent preparations were used).

Generation of Piezo1 Antisera.

Custom polyclonal antibodies to the synthetic peptide RQRRERARQERAEQ (amino acids 1454-1467 of SEQ ID NO:2) were prepared in rabbit by standard methods and affinity purified (Thermo Fisher Scientific, Openbiosystems).

TRPA1 Live-Labeling and Immunocytochemistry.

TRPA1 live-labeling and immunocytochemistry on HEK 293T cells were performed essentially as described (Schmidt et al., 2009) with the following modifications: For assessment of the specificity of Piezo1 antisera, cells were transfected with a Piezo1-IRES-EGFP construct and used 36 hrs later for immunocytochemistry as outlined below. Piezo1 antisera were used at 1:100 and detected by secondary antibodies conjugated to Alexa Fluor 546 (Invitrogen). For assessment of membrane expression of Piezo1, cells were co-transfected with a murine Trpa1-MYC/His construct and Piezo1 and used for live-labeling 36 hrs after transfection. Surface TRPA1 was labeled by incubating live HEK293T cells with TRPA1 antibodies (1:50) followed by incubation with Alexa Fluor 488 F(ab′)2 fragment of goat-anti-rabbit (1:200, Invitrogen). Cells were fixed with 2% paraformaldehyde (PFA) in PBS for 20 min, washed and incubated with an excess of non-labeled goat-anti-rabbit IgG for 1 hour to block binding sites on any remaining unbound TRPA1 antibody. Cells were then permeabilized in PBS containing 0.4% Triton X-100, blocked with normal goat serum (10% serum in PBS), and incubated with primary antibodies against Piezo1 (1:100) and c-MYC (1:100, 9E11, mouse, Santa Cruz Biotechnology), followed by secondary antibodies (Alexa Fluor 568 goat-anti-rabbit IgG and Alexa Fluor 633 goat-anti-mouse IgG; 1:200; Invitrogen).

Immunocytochemistry experiments were imaged using an Olympus (Tokyo, Japan) Fluoview 500 confocal microscope by sequential illumination using the 488 nm line of an argon laser, a HeNe green 543 nm laser and a HeNe red 633 nm laser. Merge stacked images were created using a 40× and 60×PlanAPO oil-immersion objective, the latter with a zoom of 1,5.

Real Time qPCR.

Dorsal root ganglia were freshly isolated from adult C57BL/6J wildtype mice and snap frozen on dry ice. Total RNA from DRG or siRNA transfected N2A cells (3 days after GFP co-transfection, same conditions then the one used for recordings) was extracted using Trizol treatment. Total RNA from all other tissues were purchased from Zyagen (San Diego). 500 ng total RNA was used to generate 1^{st} strand cDNA using the Quantitect reverse transcription kit (Qiagen). Real time Taqman PCR assays for mPiezo1 and mPiezo2 (assay id: Mm01241570_g1 and Mm01262433_m1) were purchased from Applied Biosystems with a FAM reporter dye and a non-fluorescent quencher. Universal TaqMan PCR master mix (20×) without AmpErase UNG (Applied Biosystems) was used. The reaction was run in the ABI 7900HT fast real time system using 1 μA of the cDNA in a 20 μA reaction according to the manufacturer's instructions in triplicate.

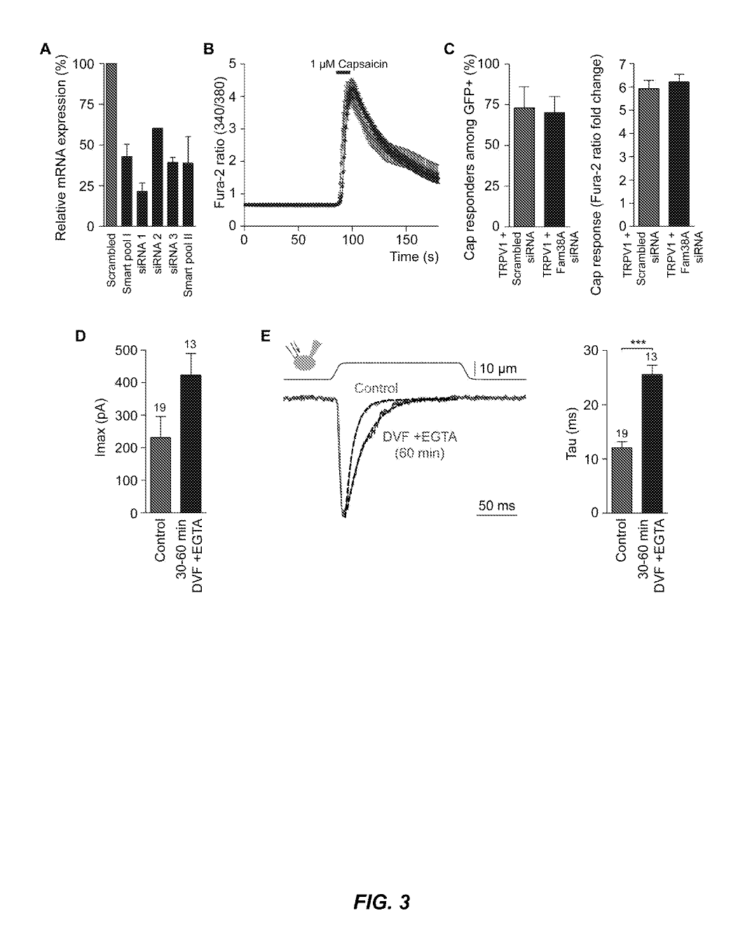

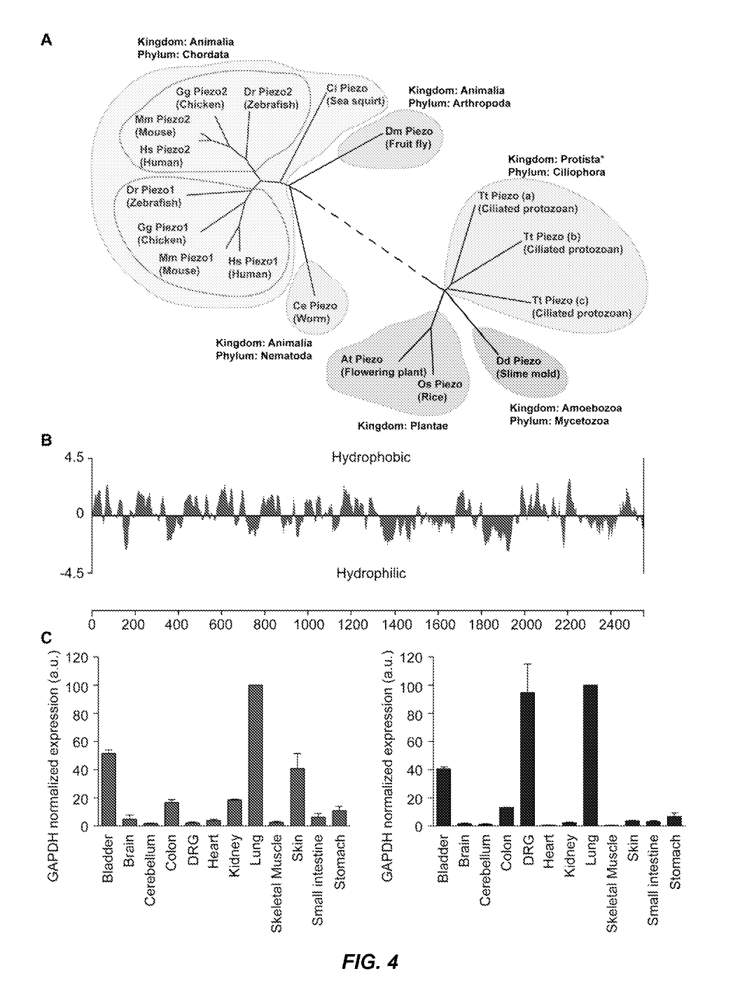

Calibrations and normalizations were done using the 2^{-ΔΔCT} method [4], where ΔΔC_{T}=((C_{T}(target gene)−C_{T} (reference gene))−(C_{T} (calibrator)−C_{T} (reference gene)). For the analysis of mRNA expression in different tissues (FIG. 4C), the target gene was mPiezo1 or mPiezo2, while the reference gene was GAPDH and the calibrator was the lung tissue. For the analysis of siRNA induced down-regulation of mRNA expression (FIG. 3A), β-actin was used as the reference gene and Scrambled siRNA transfected N2A cells was the calibrator.

In Situ Hybridization and Immunohistochemistry.

In situ hybridization and immunohistochemistry were performed as described previously (M. Chalfie, Nat Rev Mol Cell Biol 10, 44 (January, 2009)). In brief, adult male C57BL/6J mice, ages 6-16 weeks, or adult male Sprague Dawley rats, were perfused with 4% PFA and dorsal root ganglia were quickly dissected. Following post-fixation and cryoprotection in 30% sucrose, single DRG were embedded in OCT and sectioned with a cryostat at 10 μm thickness. Four different, 1000 bps cRNA sense and anti-sense probes were generated corresponding to bases-3822-4886; 4837-5849; 5922-7019 and 7102-8171. All probes were in vitro-transcribed and labeled with digoxigenin (Roche Diagnostics). For fluorometric in situ hybridizations, a peroxidase-conjugated anti-digoxygenin-POD antibody (1:500) and tyramide signal amplification (TSA; NEN) were used to detect and visualize the hybridized probes. Immunohistochemistry was performed after in situ hybridization and TSA detection. Chicken anti-NF-200 (1:1000; Abcam) and chicken anti-Peripherin (1:100; Abcam) were used on mouse DRG, while guinea pig anti-TRPV1 (1:1000; Abcam) primary antibodies were used on rat DRG (this antibody did not perform on mouse DRG). Primary antibodies were detected by secondary antibodies conjugated to Alexa Fluor 568. For colorimetric in situ hybridizations, an alkaline phosphatase anti-DIG-AP antibody (Roche; 1:500) was used followed by incubation with NBT/BCIP liquid substrate system (Sigma) for development of the dark purple color. Sections were mounted in Slow fade Gold reagent (Invitrogen) and imaged using a AX70 microscope (Olympus).

Fluorometric in situ hybridizations were used for quantitation and were imaged using an Olympus (Tokyo, Japan) Fluoview 500 confocal microscope by sequential illumination using the 488 nm line of an argon laser and the HeNe green 543 nm laser. Merge stacked images were created using a 20× and a 40×PlanAPO oil-immersion objective. Images for all experimental groups were taken using identical acquisition parameters and raw images were used for analysis with Image J (NIH). Neurons were considered Piezo2-positive if the mean fluorescence intensity (measured in arbitrary units) was higher than the mean background fluorescence plus 4 times the standard deviation measured from at least 10 random unstained cells. Only sections being at least 50 μm apart were considered to avoid double counting neurons. Neurons were considered NF200- or Peripherin-positive if the mean fluorescence intensity (measured in arbitrary units) was higher than the mean background fluorescence plus 3 times the standard deviation measured from at least 10 random unstained cells. Colorimetric in situ hybridizations of Piezo2 gave similar results (26.9% Piezo2-positive neurons, data not shown), but were not used for quantitation due to variability depending on the length of development with substrate. Only for presentation purposes brightness, contrast and levels of images were adjusted.

Molecular Cloning of Piezo1.

Primers were designed from cDNA sequence of mPiezo1 from the NCBI database (NM_{—}001037298). A 7.644 kb fragment was amplified from cDNA libraries generated from Neuro2A total RNA using primers mPiezo1 fwd (5′ atggagccgcacgtgctg 3′ (SEQ ID NO:21)) and mPiezo1 rev (5′ ctactccctctcacgtgtcca 3′(SEQ ID NO:22)) and cloned into pcDNA3.1 (−) (Invitrogen) with NotI and HindIII restriction sites. Fully sequencing this construct revealed insertion of Q at residue 156, and three amino acid changes (R147G, V228I and M1571V) compared to the NCBI sequence.

This vector was further modified to include 3′ AscI and FseI restriction sites and an IRES-GFP PCR fragment from pIRES2-EGFP (Clontech) was then inserted using these sites. The protein sequence of Piezo1 that was cloned from N2A cells (SEQ ID NO:2) is:

MEPHVLGAGLYWLLLPCTLLAASLLRFNALSLVYLLFLLLLPWLPGPSRHSIPGHTGRLLRALLCLSLLFLV
AHLAFQICLHTVPHLDQFLGQNGSLWVKVSQHIGVTRLDLKDIFNTTRLVAPDLGVLLASSLCLGLCGRLT
RKAGQSRRTQELQDDDDDDDDDDEDIDAAPAVGLKGAPALATKRRLWLASRFRVTAHWLLMTSGRTLVI
VLLALAGIAHPSAFSSIYLVVFLAICTWWSCHFPLSPLGFNTLCVMVSCFGAGHLICLYCYQTPFIQDMLPPG
NIWARLFGLKNFVDLPNYSSPNALVLNTKHAWPIYVSPGILLLLYYTATSLLKLHKSCPSELRKETPREDEE
HELELDHLEPEPQARDATQGEMPMTTEPDLDNCTVHVLTSQSPVRQRPVRPRLAELKEMSPLHGLGHLIM
DQSYVCALIAMMVWSIMYHSWLTFVLLLWACLIWTVRSRHQLAMLCSPCILLYGLTLCCLRYVWAMELP
ELPTTLGPVSLHQLGLEHTRYPCLDLGAMLLYLLTFWLLLRQFVKEKLLKKQKVPAALLEVTVADTEPTQT
QTLLRSLGELVTGIYVKYWIYVCAGMFIVVSFAGRLVVYKIVYMFLFLLCLTLFQVYYTLWRKLLRVFWW
LVVAYTMLVLIAVYTFQFQDFPTYWRNLTGFTDEQLGDLGLEQFSVSELFSSILIPGFFLLACILQLHYFHRP
FMQLTDLEHVPPPGTRHPRWAHRQDAVSEAPLLEHQEEEEVFREDGQSMDGPHQATQVPEGTASKWGLV
ADRLLDLAASFSAVLTRIQVFVRRLLELHVFKLVALYTVWVALKEVSVMNLLLVVLWAFALPYPRFRPMA
SCLSTVWTCIIIVCKMLYQLKIVNPHEYSSNCTEPFPNNTNLQPLEINQSLLYRGPVDPANWFGVRKGYPNL
GYIQNHLQILLLLVFEAVVYRRQEHYRRQHQQAPLPAQAVCADGTRQRLDQDLLSCLKYFINFFFYKFGLE
ICFLMAVNVIGQRMNFMVILHGCWLVAILTRRRREAIARLWPNYCLFLTLFLLYQYLLCLGMPPALCIDYP
WRWSKAIPMNSALIKWLYLPDFFRAPNSTNLISDFLLLLCASQQWQVFSAERTEEWQRMAGINTDHLEPLR
GEPNPIPNFIHCRSYLDMLKVAVFRYLFWLVLVVVFVAGATRISIFGLGYLLACFYLLLFGTTLLQKDTRAQ
LVLWDCLILYNVTVIISKNMLSLLSCVFVEQMQSNFCWVIQLFSLVCTVKGYYDPKEMMTRDRDCLLPVEE
AGIIWDSICFFFLLLQRRIFLSHYFLHVSADLKATALQASRGFALYNAANLKSINFHRQIEEKSLAQLKRQMK
RIRAKQEKYRQSQASRGQLQSKDPQDPSQEPGPDSPGGSSPPRRQWWRPWLDHATVIHSGDYFLFESDSEE
EEEALPEDPRPAAQSAFQMAYQAWVTNAQTVLRQRRERARQERAEQLASGGDLNPDVEPVDVPEDEMAG
RSHMMQRVLSTMQFLWVLGQATVDGLTRWLRAFTKHHRTMSDVLCAERYLLTQELLRVGEVRRGVLDQ
LYVGEDEATLSGPVETRDGPSTASSGLGAEEPLSSMTDDTSSPLSTGYNTRSGSEEIVTDAGDLQAGTSLHG
SQELLANARTRMRTASELLLDRRLHIPELEEAERFEAQQGRTLRLLRAGYQCVAAHSELLCYFIIILNHMVT
ASAASLVLPVLVFLWAMLTIPRPSKRFWMTAIVFTEVMVVTKYLFQFGFFPWNSYVVLRRYENKPYFPPRI
LGLEKTDSYIKYDLVQLMALFFHRSQLLCYGLWDHEEDRYPKDHCRSSVKDREAKEEPEAKLESQSETGT
GHPKEPVLAGTPRDHIQGKGSIRSKDVIQDPPEDLKPRHTRHISIRFRRRKETPGPKGTAVMETEHEEGEGKE
TTERKRPRHTQEKSKFRERMKAAGRRLQSFCVSLAQSFYQPLQRFFHDILHTKYRAATDVYALMFLADIVD
IIIIIFGFWAFGKHSAATDIASSLSDDQVPQAFLFMLLVQFGTMVIDRALYLRKTVLGKLAFQVVLVVAIHIW
MFFILPAVTERMFSQNAVAQLWYFVKCIYFALSAYQIRCGYPTRILGNFLTKKYNHLNLFLFQGFRLVPFLV
ELRAVMDWVWTDTTLSLSNWMCVEDIYANIFIIKCSRETEKKYPQPKGQKKKKIVKYGMGGLIILFLIAIIW
FPLLFMSLIRSVVGVVNQPIDVTVTLKLGGYEPLFTMSAQQPSIVPFTPQAYEELSQQFDPYPLAMQFISQYS
PEDIVTAQIEGSSGALWRISPPSRAQMKQELYNGTADITLRFTWNFQRDLAKGGTVEYTNEKHTLELAPNST
ARRQLAQLLEGRPDQSVVIPHLFPKYIRAPNGPEANPVKQLQPDEEEDYLGVRIQLRREQVGTGASGEQAG
TKASDFLEWWVIELQDCKADCNLLPMVIFSDKVSPPSLGFLAGYGIVGLYVSIVLVVGKFVRGFFSEISHSI
MFEELPCVDRILKLCQDIFLVRETRELELEEELYAKLIFLYRSPETMIKWTRERE

Molecular Cloning of Piezo2. Primers were designed from cDNA sequence of Piezo2 from the NCBI database (NM_{—}001039485). An 8.469 kb fragment was amplified from cDNA libraries generated from adult C57BL/6J DRG total RNA using primers mPiezo2 fwd (5′ atggcttcggaagtggtgtgc 3′ (SEQ ID NO:23)) and mPiezo2 rev (5′ tcagtttgttttttctctagtccac 3′ (SEQ ID NO:24)) and cloned into pCMV-Sport6 (Invitrogen) with KpnI and NotI restriction sites. Sequencing of the cloned mPiezo2 gene from DRG revealed differences from the NCBI annotation where three regions of amino acid insertions were not correctly assigned as exons (628E, 14 residues at 833 and 56 residues at 1751). The protein sequence of Piezo2 that we cloned from mouse DRG (SEQ ID NO:4) is:

MASEVVCGLIFRLLLPICLAVACAFRYNGLSFVYLIYLLLIPLFSEPTKATMQGHTGRLLQSLCITSLSFLLLHI
IFHITLASLEAQHRITPAYNCSTWEKTFRQIGFESLKGADAGNGIRVFVPDIGMFIASLTIWLVCRTIVKKPDT
EEIAQLNSECENEELAGGEKMDSEEALIYEEDLDGEEGMEGELEESTKLKILRRFASVASKLKEFIGNMITTA
GKVVVTILLGSSGMMLPSLTSAVYFFVFLGLCTWWSWCRTFDPLLFGCLCVLLAIFTAGHLIGLYLYQFQFF
QEAVPPNDYYARLFGIKSVIQTDCASTWKIIVNPDLSWYHHANPILLLVMYYTLATLIRIWLQEPLVQEEMA
KEDEGALDCSSNQNTAERRRSLWYATQYPTDERKLLSMTQDDYKPSDGLLVTVNGNPVDYHTIHPSLPIEN
GPAKTDLYTTPQYRWEPSEESSEKKEEEEDKREDSEGEGSQEEKRSVRMHAMVAVFQFIMKQSYICALIAM
MAWSITYHSWLTFVLLIWSCTLWMIRNRRKYAMISSPFMVVYANLLLVLQYIWSFELPEIKKVPGFLEKKE
PGELASKILFTITFWLLLRQHLTEQKALREKEALLSEVKIGSQELEEKEDEELQDVQVEGEPTEKEEEEEEEI
KEERHEVKKEEEEEVEEDDDQDIMKVLGNLVVALFIKYWIYVCGGMFFFVSFEGKIVMYKIIYMVLFLFCV
ALYQVHYEWWRKILKYFWMSVVIYTMLVLIFIYTYQFENFPGLWQNMTGLKKEKLEDLGLKQFTVAELFT
RIFIPTSFLLVCILHLHYFHDRFLELTDLKSIPSKEDNTIYSHAKVNGRVYLIINRLAHPEGSLPDLAIMNMTAS
LDKPEVQKLAESGEERPEECVKKTEKGEAGKDSDESEEEEDEEEESEEEESSDLRNKWHLVIDRLTVLFLKF
LEYFHKLQVFMWWILELHIIKIVSSYIIWVTVKEVSLFNYVFLISWAFALPYAKLRRAASSVCTVWTCVIIVC
KMLYQLQTIKPENFSVNCSLPNENQTNIPLHELNKSLLYSAPVDPTEWVGLRKSSPLLVYLRNNLLMLAILA
FEVTVYRHQEYYRGRNNLTAPVSKTIFHDITRLHLDDGLINCAKYFVNYFFYKFGLETCFLMSVNVIGQRM
DFYAMIHACWLIGVLYRRRRKAIAEVWPKYCCFLACIITFQYFVCIGIPPAPCRDYPWRFKGAYFNDNIIKW
LYFPDFIVRPNPVFLVYDFMLLLCASLQRQIFEDENKAAVRIMAGDNVEICMNLDAASFSQHNPVPDFIHCR
SYLDMSKVIIFSYLFWFVLTIIFITGTTRISIFCMGYLVACFYFLLFGGDLLLKPIKSILRYWDWLIAYNVFVIT
MKNILSIGACGYIGALVRNSCWLIQAFSLACTVKGYQMPEDDSRCKLPSGEAGIIWDSICFAFLLLQRRVFM
SYYFLHVVADIKASQILASRGAELFQATIVKAVKARIEEEKKSMDQLKRQMDRIKARQQKYKKGKERMLS
LTQESGEGQDIQKVSEEDDEREADKQKAKGKKKQWWRPWVDHASMVRSGDYYLFETDSEEEEEEELKKE
DEEPPRKSAFQFVYQAWITDPKTALRQRRKEKKKLAREEQKERRKGSGDGPVEWEDREDEPVKKKSDGPD
NIIKRIFNILKFTWVLFLATVDSFTTWLNSISREHIDISTVLRIERCMLTREIKKGNVPTRESIHMYYQNHIMNL
SRESGLDTIDEHSGAGSRAQAAHRMDSLDSRDSISSCYTEATLLISRQSTLDDLDGQDPVPKTSERARPRLR
KMFSLDMSSSSADSGSVASSEPTQCTMLYSRQGTTETIEEVEAEAEEEVVEGLEPELHDAEEKEYAAEYEA
GVEEISLTPDEELPQFSTDDCEAPPSYSKAVSFEHLSFASQDDSGAKNHMVVSPDDSRTDKLESSILPPLTHE
LTASDLLMSKMFHDDELEESEKFYVDQPRFLLLFYAMYNTLVARSEMVCYFVIILNHMTSASIITLLLPILIF
LWAMLSVPRPSRRFWMMAIVYTEVAIVVKYFFQFGFFPWNKDLEIYKERPYFPPNIIGVEKKEGYVLYDLI
QLLALFFHRSILKCHGLWDEDDIVDSNTDKEGSDDELSLDQGRRGSSDSLKSINLAASVESVHVTFPEQPAA
IRRKRSCSSSQISPRSSFSSNRSKRGSTSTRNSSQKGSSVLSLKQKSKRELYMEKLQEHLIKAKAFTIKKTLQI
YVPIRQFFYDLIHPDYSAVTDVYVLMFLADTVDFIIIVFGFWAFGKHSAAADITSSLSEDQVPGPFLVMVLIQ
FGTMVVDRALYLRKTVLGKVIFQVILVFGIHFWMFFILPGVTERKFSQNLVAQLWYFVKCVYFGLSAYQIR
CGYPTRVLGNFLTKSYNYVNLFLFQGFRLVPFLTELRAVMDWVWTDTTLSLSSWICVEDIYAHIFILKCWR
ESEKRYPQPRGQKKKKAVKYGMGGMIIVLLICIVWFPLLFMSLIKSVAGVINQPLDVSVTITLGGYQPIFTM
SAQQSQLKVMDNSKYNEFLKSFGPNSGAMQFLENYEREDVTVAELEGNSNSLWTISPPSKQKMIQELTDPN
SCFSVVFSWSIQRNMTLGAKAEIATDKLSFPLAVATRNSIAKMIAGNDTESSNTPVTIEKIYPYYVKAPSDSN
SKPIKQLLSENNFMNITIILFRDNVTKSNSEWWVLNLTGSRIFNQGSQALELVVFNDKVSPPSLGFLAGYGIM
GLYASVVLVIGKFVREFFSGISHSIMFEELPNVDRILKLCTDIFLVRETGELELEEDLYAKLIFLYRSPETMIK
WTREKTN

Phylogenic Analysis.

Accession numbers of Piezo sequences used to make the phylogenic analysis are given with the number of TM domain predicted using TMHMM2 program. For some species, multiple predicted gene sequences were fused to obtain a complete sequence.

### Hs Piezo1 (Homo Sapiens): NP_{—}001136336.2, 2521aa (31 TM)

Mm Piezo1 (Mus musculus): NP_{—}001032375.1, 2546aa (30 TM)
Gg Piezo1 (Gallus gallus): XP_{—}414209.2, 1718aa; XP_{—}423106.2, 217aa (25 TM)

### Dr Piezo1 (Danio Rerio): XP_{—}696355.4, 2538aa (29 TM)

### Hs Piezo2 (Homo Sapiens): NP_{—}071351.2, 2752aa (35 TM)

Mm Piezo2 (Mus musculus): NP_{—}001034574.3, 2753aa (34 TM)
Gg Piezo2 (Gallus gallus): XP_{—}419138.2, 3080aa (33 TM)

### Dr Piezo2 (Danio Rerio): XP_{—}002666625., 2102aa (24 TM)

Ci Piezo (Ciona intestinalis): XP_{—}002122901.1, 1669aa; XP_{—}002128850.1, 591aa (33 TM)
Dm Piezo (Drosophila melanogaster): NP_{—}001036346.3, 2671 as (36 TM)
Ce Piezo (Caenorhabditis elegans): NP_{—}501648.2, 800a; NP_{—}501647.2, 1843 (33 TM)
Dd Piezo (Dictyostelium discoideum): XP_{—}640187, 3080 aa (35 TM)
At Piezo (Arabidopsis thaliana): NP_{—}182327.5, 2440 aa (28 TM)
Os Piezo (Oryza sativa-japonica group): NP_{—}001043105.1, 2196aa (24 TM)
Tt Piezo i (Tetrahymena thermophila): XP_{—}976967.1, 4690aa (30 TM)
Tt Piezo ii (Tetrahymena thermophila): XP_{—}001021704.1, 4136aa (29 TM)
Tt Piezo iii (Tetrahymena thermophila): XP_{—}001017682.1, 2636aa (26 TM)

Data Analysis.

Data in all figures are shown as mean±SEM. Unless otherwise stated, statistical significance was evaluated using unpaired two-tailed Student's t-test for comparing difference between two samples. Unpaired two-tailed Student's t-test with Welch correction was used when variances where significantly different. * p<0.05, ** p<0.01, *** p<0.001.

### Example 2

### Neuro2A Cells Express MA Currents

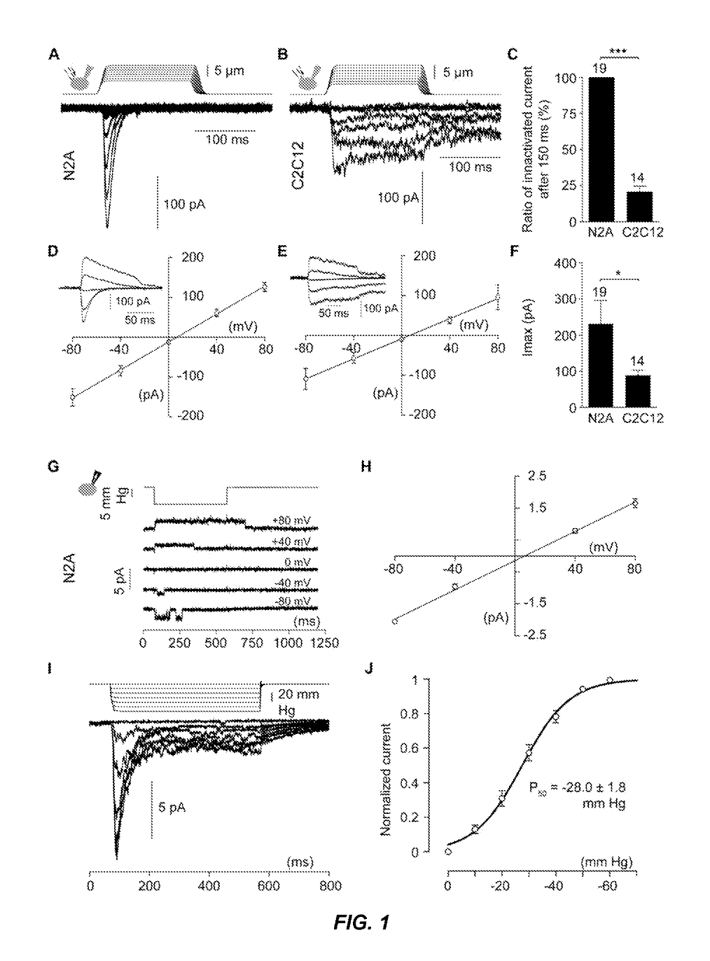

To identify proteins involved in mechanotransduction, a cell line was sought that expresses a robust MA current similar to those recorded from primary cells (B. Coste, M. Crest, P. Delmas, J Gen Physiol 129, 57 (January, 2007)). Several mouse and rat cell lines (Neuro2A, C2C12, NIH/3T3, Min-6, 50B11, F11, PC12) were screened by patch-clamp in the whole cell configuration using a mechanical stimulus consisting of a piezo-electrically driven glass probe (G. C. McCarter, D. B. Reichling, J. D. Levine, Neurosci Lett 273, 179 (Oct. 8, 1999); B. Coste, M. Crest, P. Delmas, J Gen Physiol 129, 57 (January, 2007); L. J. Drew, J. N. Wood, P. Cesare, J Neurosci 22, RC228 (Jun. 15, 2002)). Neuro2A (N2A) mouse neuroblastoma cell line expressed the most consistent MA currents with considerable kinetics of adaptation (FIG. 1A). In comparison, as a representative of MA currents recorded in other cell lines, the C2C12 mouse myoblast cell line expressed MA currents with slower kinetics of inactivation (FIG. 1B). All the N2A MA currents recorded at a holding potential of −80 mV are inactivated at the end of a 150 ms mechanical stimulation pulse, whereas the C2C12 MA currents are only partially inactivated (FIG. 1C). These currents share kinetic properties with some currents expressed in DRG neurons, where rapidly-adapting and slowly-adapting MA currents are described (B. Coste et al., J Gen Physiol 129, 57 (January, 2007); L. J. Drew et al., J Physiol 556, 691 (May 1, 2004); L. J. Drew et al., PLoS ONE 2, e515 (2007); J. Hu, G. R. Lewin, J Physiol 577, 815 (Dec. 15, 2006); C. Wetzel et al., Nature 445, 206 (Jan. 11, 2007)) (Table 1 and see below). The difference in inactivation kinetics suggests that distinct components of the mechanotransduction apparatus are expressed in these cell lines; however, it is also possible that this difference in inactivation kinetics is due to indirect factors, such as membrane distensibility of the cell lines. Current-voltage relationships of N2A and C2C12 MA currents are linear between −80 and +80 mV with reversal potentials (Eᵣₑᵥ) at +6.6 and +6.7 mV, respectively, and inward currents are suppressed with NMDG-chloride external solutions (data not shown), suggesting cationic non-selective permeability (FIG. 1D-E). Finally, the amplitude of MA currents expressed in N2A cells is approximately two-fold larger than the C2C12 MA currents (FIG. 1F).

TABLE 1
Kinetics of inactivation in untreated and Piezo1-treated cell lines

| | | Whole-cell mechanical stimulation | | | Cell-attached stretch | |
|---|---|---|---|---|---|---|
| | | Threshold | Tau inac | Imax (−80 mV) | P50 | Imax (−80 mV) |
| | | (μm) | (ms) | (pA) | (mm Hg) | (pA) |
| N2A | control | 5.4 ± 0.3 | 12.3 ± 0.6 | −194.3 ± 34.7 | −28.0 ± 1.8 | −8.1 ± 2.0 |
| | | (n = 71) | (n = 74) | (n = 89) | (n = 21) | (n = 28) |
| | Piezo1 | n.d. | n.d. | −13.8 ± 3.2 | n.d. | −1.3 ± 0.5 |
| | siRNA | | | (n = 67) | | (n = 27) |
| | Piezo1 | 3.7 ± 0.7 | 15.3 ± 1.5 | −3568 ± 567.6 | −28.1 ± 2.8 | −68.6 ± 7.3 |
| | cDNA | (n = 16) | (n = 16) | (n = 16) | (n = 13) | (n = 13) |
| C2C12 | control | 3.3 ± 0.4 | 129.8 ± 55.4 | −101.5 ± 19.7 | n.d. | n.d. |
| | | (n = 23) | (n = 21) | (n = 25) | | |
| | Piezo1 | 3.0 ± 0.3 | 15.6 ± 0.9 | −2482 ± 384.4 | n.d. | n.d. |
| | cDNA | (n = 10) | (n = 10) | (n = 10) | | |
| HEK293T | control | 6.0 ± 0.9 | 88.7 ± 26.0 | −88.5 ± 19.7 | n.d. | −1.1 ± 0.2 |
| | | (n = 9) | (n = 7) | (n = 10) | | (n = 18) |
| | Piezo1 | 2.6 ± 0.7 | 16.5 ± 1.4 | −3696 ± 641.1 | −31.2 ± 3.5 | −43.0 ± 12.8 |
| | cDNA | (n = 10) | (n = 10) | (n = 10) | (n = 11) | (n = 17) |

The N2A MA currents were further characterized by using patch-membrane stretch stimulation in cell-attached mode (Besch et al., Pflugers Arch 445, 161 (October, 2002)). Brief negative pressure pulses evoked opening of endogenous channels (FIG. 1G), with a single-channel conductance of 22.9±1.4 pS and Eᵣₑᵥ of +6.2 mV (FIG. 1H). Increasing the magnitude of pressure pulses induced gradual and reversible opening of these MA channels (FIG. 1I). The current-pressure relationship is characterized by maximal opening at −60 mm Hg, with a pressure for half-maximal activation (P₅₀) of −28.0±1.8 mm Hg (FIG. 1J). These conductance and P₅₀ values are similar to the properties of reported stretch-activated channels (Cho et al., Eur J Neurosci 23, 2543 (May, 2006); Earley et al., Circ Res 95, 922 (Oct. 29, 2004); Sharif-Naeini et al., J Mol Cell Cardiol 48, 83 (January, 2010)).

### Example 3

### Piezo1 (Fam38A) is Required for MA Currents of N2A Cells

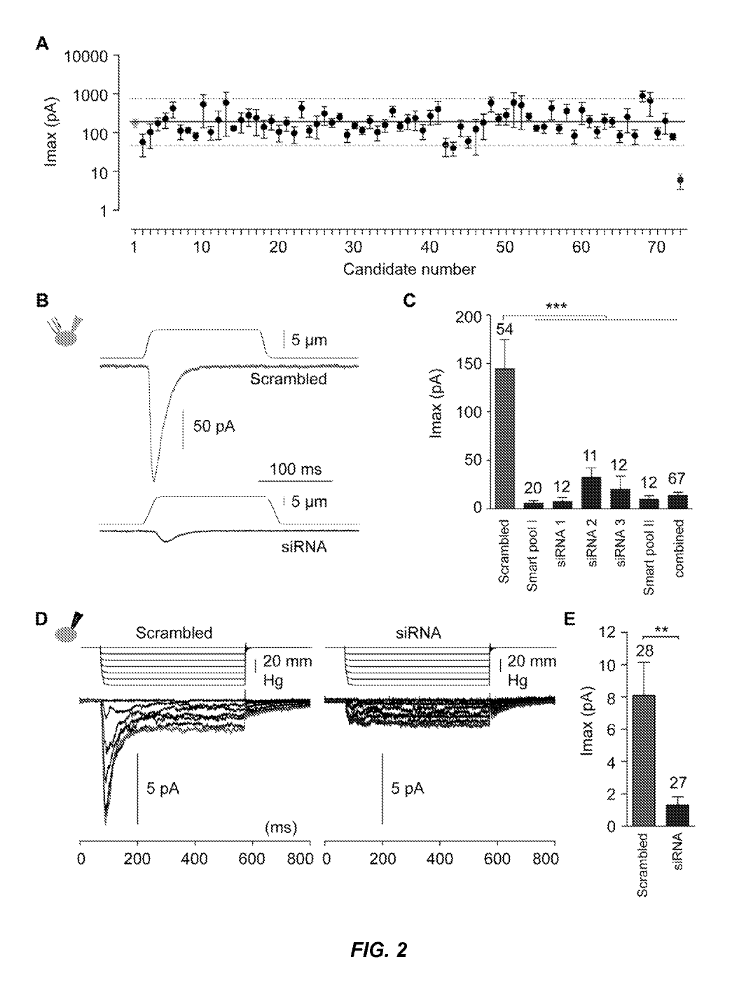

To generate a list of candidate MA ion channels in N2A, gene expression profiling was carried out on N2A and other mouse cell lines tested, and transcripts were focused on that are enriched in N2A cells using a combination of criteria. Proteins predicted to span the membrane at least two times (a characteristic shared by all ion channels) were selected. This list was prioritized by picking either known cation channels, or proteins with unknown function. Each candidate was tested using siRNA knockdown in N2A cells, measuring MA currents via piezo-driven pressure stimulation in the whole cell mode. Knockdown of Fam38A (Family with sequence similarity 38), the 73^{rd} candidate, caused a pronounced decrease of MA currents (FIG. 2A-B, Table 2). In follow-up experiments, robust attenuation of MA currents was observed with different siRNAs directed against this gene (FIG. 2C). All the siRNAs tested significantly decreased target transcripts as assayed by qPCR (FIG. 3A).

TABLE 2
Candidate proteins for siRNA knockdown

| No: | siRNA: |
|---|---|
| 1 | Scrambled |
| 2 | 2400010G15Rik |
| 3 | 2410015B03Rik |
| 4 | 4833424O15Rik |
| 5 | Tmem129 |
| 6 | 1500016O10Rik |
| 7 | 2810432L12Rik |
| 8 | 3632451O06Rik |
| 9 | 4930500O05Rik |
| 10 | 9330182L06Rik |
| 11 | A830020B06Rik |
| 12 | Accn2 |
| 13 | Al848100 |
| 14 | B430119L13Rik |
| 15 | BC042720 |
| 16 | BC062109 |
| 17 | Chrna3 |
| 18 | Creld1 |
| 19 | D630045J12Rik |
| 20 | Emb |
| 21 | Gpr173 |
| 22 | Grik5 |
| 23 | Htr3a |
| 24 | Htr3b |
| 25 | Josd2 |
| 26 | Leprotl1 |
| 27 | Lphn1 |
| 28 | Mcam |
| 29 | Mfap3 |
| 30 | Npal2 |
| 31 | Npal3 |
| 32 | Nrsn1-Vmp |
| 33 | P2X3 |
| 34 | Pcdh1 |
| 35 | Pcnxl2 |
| 36 | PKD1L2 |
| 37 | Prrt3 |
| 38 | Punc |
| 39 | Reep2 |
| 40 | Rom1 |
| 41 | Sfxn5 |
| 42 | Slc16a6 |
| 43 | Slc47a1 |
| 44 | Slc7a3 |
| 45 | Slc7a7 |
| 46 | Slc8a1 |
| 47 | TM6sf1 |
| 48 | Tmc6 |
| 49 | Tmem108 |
| 50 | Tmem136 |
| 51 | Tmem161a |
| 52 | Tmem164 |
| 53 | Tmem16f |
| 54 | TMEM176a |
| 55 | TMEM176b |
| 56 | Tmem181 |
| 57 | Tmem20 |
| 58 | Tmem41a |
| 59 | Tmem54 |
| 60 | Tmem56 |
| 61 | Tmem74 |
| 62 | Tmhs |
| 63 | Tmtc1 |
| 64 | Tmtc2 |
| 65 | TRPM2 |
| 66 | TRPML3 |
| 67 | TRPV2 |
| 68 | Tspan13 |
| 69 | Tspan18 |
| 70 | Tspan2 |
| 71 | Tspan33 |
| 72 | Xkr6 |
| 73 | Fam38a |

Given that Fam38A encodes a protein required for the expression of ion channels activated by pressure, this gene was named Piezo1, from the Greek “πiεση” (piesi) meaning pressure. To test whether knocking down Piezo1 impairs general cell signaling or viability, N2A cells were transfected with TRPV1 cDNA and either scrambled or Piezo1 siRNA. No differences were observed in capsaicin responses (FIG. 3B-C). Next, experiments were conducted to determine if Piezo1 is also required for N2A MA currents elicited by patch membrane stretch (FIG. 2D-E). Once again, strong knockdown of MA currents was observed with siRNA against Piezo1. This suggests that Piezo1 is required for the expression of the MA currents recorded using either of the two mechanostimulation protocols.

Very little is known about mammalian Piezo1 (KIAA0233, Fam38A, Mib). Its expression is induced in senile plaque-associated astrocytes (K. Satoh et al., Brain Res 1108, 19 (Sep. 7, 2006)), and the protein has been suggested to be involved in integrin activation (B. J. McHugh et al., J Cell Sci 123, 51 (Jan. 1, 2010)). Extracellular perfusion of divalent-free and 5 mM EGTA solution for 30-60 min, which disrupts integrin function (R. O. Hynes, Cell 110, 673 (Sep. 20, 2002)), did not suppress MA currents (FIG. 3D-E). Thus it is unlikely that Piezo1 siRNA blocks MA currents through integrin modulation. However, it is possible that mechanical activation of Piezo1 could lead to integrin activation and various downstream consequences including cell adhesion, division, and migration.

### Example 4

### Piezos are Large Transmembrane Proteins Conserved Among Various Species

Piezo proteins are present in non-mammalian species, none reported as characterized. Many animal, plant, and other eukaryotic species contain a single Piezo (FIG. 4A). Vertebrates (mammals, birds, fish) have two members, Piezo1 (Fam38A) and Piezo2 (Fam38B). However, the early chordate Ciona has a single member. Multiple Piezos are also present in the Ciliophora kingdom: Tetrahymena thermophila has three members; Paramecium Tetraurelia, six (not shown). No clear homologs were identified in yeast or bacteria. The secondary structure and overall length of Piezo proteins are moderately conserved, while homology to other proteins is minimal. As assayed by the TMHMM2 program, all have between 24-36 predicted transmembrane domains (with variability perhaps due to inaccurate cDNA or transmembrane prediction). The predicted proteins are 2100-4700 amino acids, and the transmembrane domains are located throughout the putative protein, as illustrated by the hydrophobicity plot of mouse Piezo1 (FIG. 4B).

The expression profile of Piezo1 determined by qPCR (FIG. 4C) includes robust expression in bladder, colon, kidney, lung and skin, and low expression in the other tissues tested including DRG sensory neurons. This pattern agrees with Northern blot expression analysis in rat (K. Satoh et al., Brain Res 1108, 19 (Sep. 7, 2006)). Bladder, colon, and lung undergo mechanotransduction related to visceral pain (G. Burnstock, Mol Pain 5, 69 (2009)), and primary cilia in the kidney sense urinary flux (L. Rodat-Despoix, P. Delmas, Pflugers Arch 458, 179 (May, 2009)). The low level of mRNA in DRG suggests that Piezo1 may not account for MA currents observed there (B. Coste, M. Crest, P. Delmas, J Gen Physiol 129, 57 (January, 2007); L. J. Drew, J. N. Wood, P. Cesare, J Neurosci 22, RC228 (Jun. 15, 2002); J. Hu, G. R. Lewin, J Physiol 577, 815 (Dec. 15, 2006)), but Piezo1 is expressed in the skin, another putative site of somatosensation. Piezo2 expression is observed in bladder, colon and lung as well, but less in kidney or skin. Interestingly, very strong expression of Piezo2 is observed in DRG sensory neurons, suggesting a potential role in somatosensory mechanotransduction (see below).

### Example 5

### Piezo1 Induces MA Currents in Various Cell Types

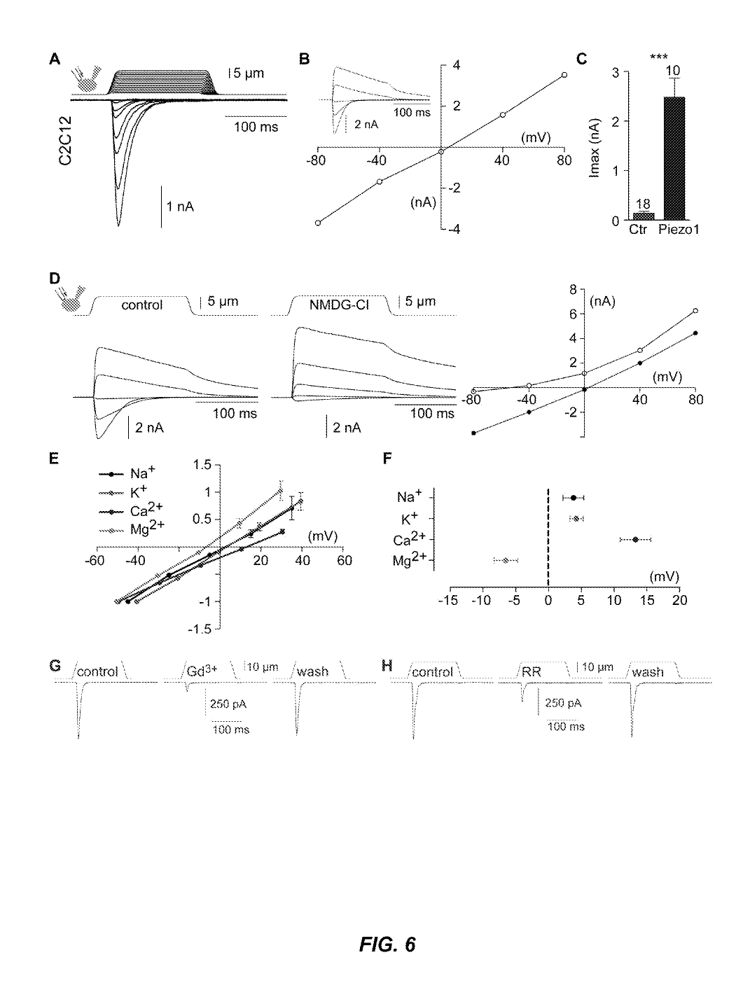

The full-length Piezo1 from N2A cells was cloned into the pIRES2-EGFP vector. Electrophysiological recordings of MA currents in the whole-cell mode were performed 12-48 hours after transfection. Piezo1 transfected cells, but not mock-transfected cells, showed large MA currents in N2A, HEK293T (FIG. 5A-F) and C2C12 cell-lines (FIG. 6A-C). In all cells overexpressing Piezo1, the MA current-voltage relationships were similar to endogenous N2A MA currents (FIGS. 5B, 5E, 6B, and 1D), with E, mV. The threshold of activation and the time constant for inactivation of MA currents elicited in Piezo1 overexpressing cells is similar in all three cell lines tested (see Table 1). Next, the ionic selectivity of Piezo1-induced MA currents was characterized. Substituting the non-permeant cation NMDG in the extracellular bathing solution suppressed inward MA currents, demonstrating that this channel activity is cationic in nature (FIG. 6D). The ionic selectivity was further examined by recording with CsCl-only internal solutions and various cations in the bath. Na⁺, K⁺, Ca²⁺ and Mg²⁺ ions were all able to permeate, with a slight preference for Ca²⁺ (FIG. 5G and FIG. 6E-F). Moreover, 30 μM of ruthenium red and gadolinium, known blockers of many cationic MA currents (L. J. Drew, J. N. Wood, P. Cesare, J Neurosci 22, RC228 (Jun. 15, 2002); J. Hao et al., in Mechanosensitivity of the Nervous System, I. K. e. A. Kamkin, Ed. (Springer Netherlands, 2008), vol. 2, pp. 51-67), blocked 74.6±2.5% (n=6) and 84.3±3.8% (n=5) of Piezo1-induced MA current, respectively (FIGS. 5H and 6G-H).

Next, patch-membrane stretch stimulation was used in cell-attached mode to assay Piezo1-transfected cells (FIG. 5I-N). Overexpression of Piezo1 in N2A and HEK293T cells gave rise to large currents elicited by negative pressure pulses (FIGS. 5I, 5L). The MA current-pressure relationships in cells overexpressing Piezo1 and in endogenous N2A cells is similar, with P₅₀ of −28.1±2.8 and −31.2±3.5 mm Hg in N2A and HEK293T overexpressing cells, respectively (FIGS. 5J, 5M, and 1J). No channel activity similar to N2A endogenous MA channels was detected in vector-only transfected HEK293T cells (data not shown). Therefore, Piezo1 overexpression induces MA currents elicited both by piezoelectrically-driven glass probe stimulation and by patch-membrane stretch in all the cell lines tested. This is the first demonstration that these two different mechanostimulation protocols activate the same MA channels.

### Example 6

### Piezo2 Induces MA Currents Distinct from Piezo1-Induced Currents

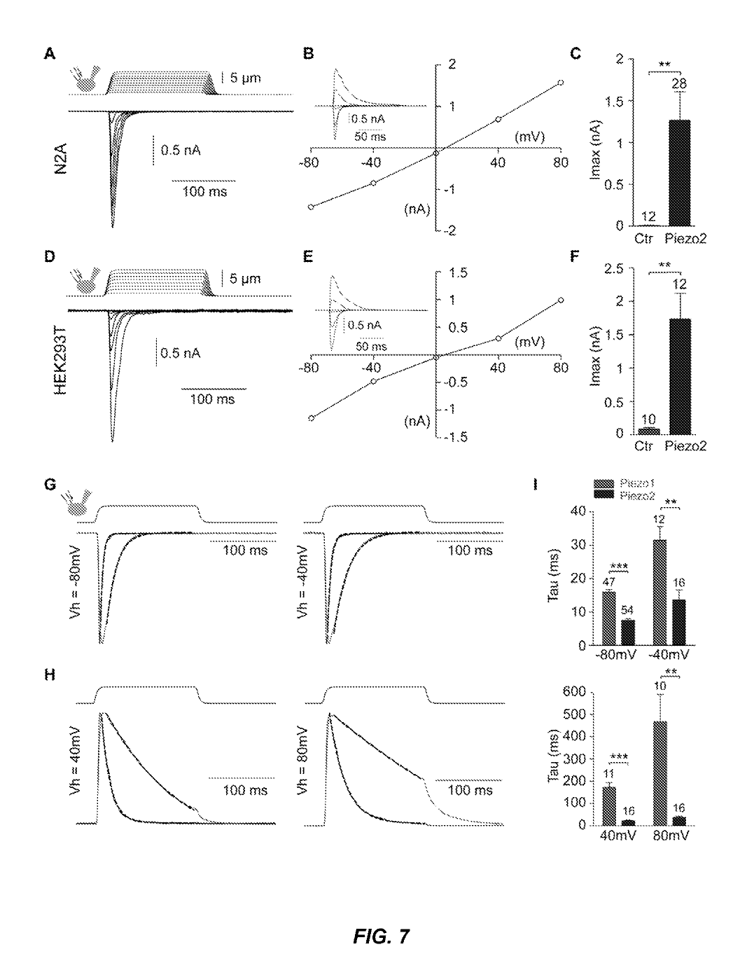

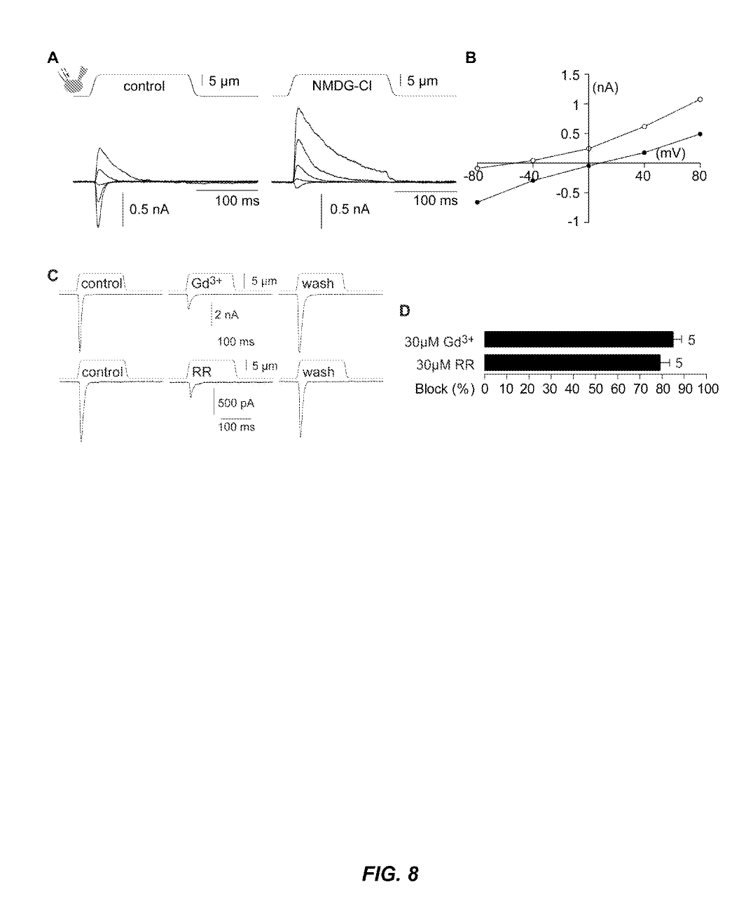

The full-length Piezo2 was cloned from DRG neurons and its mechanosensitivity upon heterologous expression was tested. Piezo2/GFP co-transfected, but not mock-transfected cells, revealed large MA currents in N2A (co-transfected with Piezo1 siRNA to suppress endogenous MA currents) and HEK293T cells (FIG. 7A-F). The Piezo2-induced MA current-voltage relationship is linear between −80 and +80 mV (FIGS. 7B, 7E), with a reversal potential (Eᵣₑᵥ) of +6.3±0.4 mV (n=3) and +8.7±1.5 mV (n=7) in N2A and HEK293T cells, respectively, suggesting relatively non-selective cationic conductance. Similar to Piezo1, Piezo2-induced currents were suppressed by the non-permeant cation NMDG and inhibited by gadolinium and ruthenium red [85.0±3.7% (n=5) and 79.2±4.2% (n=5), respectively] (FIG. 8). These characteristics fit the profile of MA currents recorded from DRG neurons (G. C. McCarter et al., Neurosci Lett 273, 179 (Oct. 8, 1999); L. J. Drew et al., J Neurosci 22, RC228 (Jun. 15, 2002); but see also J. Hu, G. R. Lewin, J Physiol 577, 815 (Dec. 15, 2006)).

The inactivation kinetics of heterologously expressed Piezo2-induced MA currents are best-fitted with a mono-exponential equation, and calculated time constants for inactivation (τ_{inac}) are relatively fast in both N2A (6.8±0.7 ms, n=27) and HEK293T (7.3±0.7, n=11) cells when measured at −80 mV. Furthermore, the kinetics of inactivation of Piezo2-induced MA currents are faster than Piezo1-induced MA currents, both for inward (FIG. 7G) and outward (FIG. 7H) currents, and at all holding potentials tested (FIG. 7I). These differences are not due to higher Ca²⁺ permeability, since the Ca²⁺ selectivity of Piezo1- and Piezo2-induced MA currents are similar (data not shown). Therefore, Piezo1 and Piezo2 confer unique channel properties. Initial attempts to further characterize properties of Piezo2-induced MA currents using negative-pressure stimulations of membrane patches in cell-attached mode could not detect Piezo2-dependent channel activity (not shown, 21 patches).

### Example 7

### Piezo1 is detected at the plasma membrane

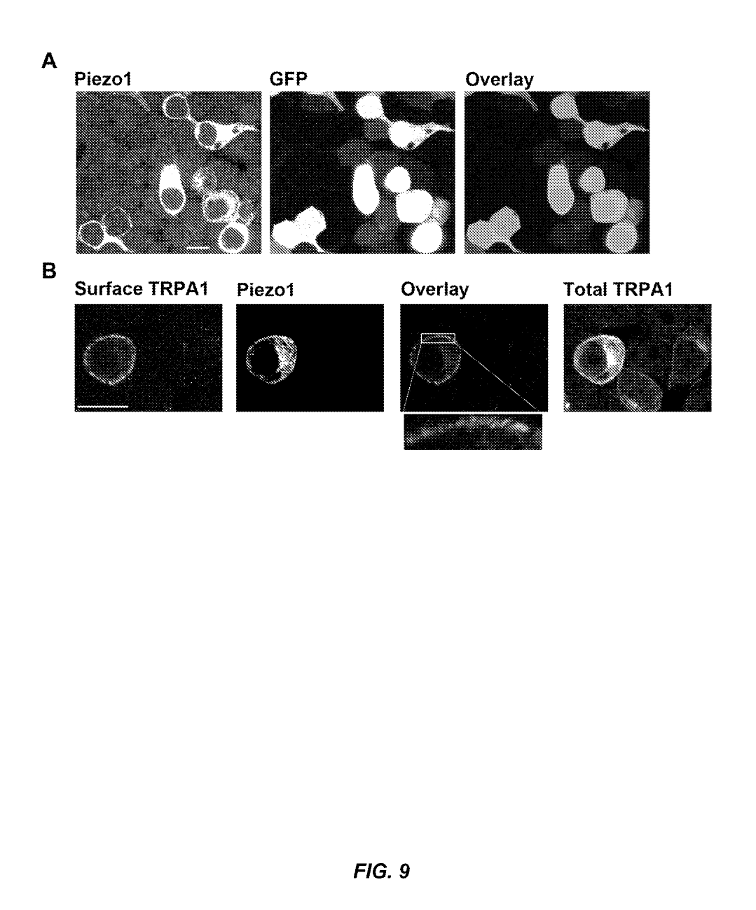

The results above suggest that Piezo1 and 2 are components of mechanotransduction complexes and therefore should be present at the plasma membrane. Previous reports have shown expression of Fam38A (Piezo1) in the endoplasmic reticulum (K. Satoh et al., Brain Res 1108, 19 (Sep. 7, 2006); B. J. McHugh et al., J Cell Sci 123, 51 (Jan. 1, 2010)). A peptide antibody against mouse Piezo1 was generated. This antibody specifically recognized Piezo1-transfected HEK293T cells, but not naive HEK293T cells (FIG. 9A). In cells transfected with Piezo1 and TRPA1, an ion channel known to be expressed at the plasma membrane, co-staining of Piezo1 was observed with live-labelled surface TRPA1 (M. Schmidt, A. E. Dubin, M. J. Petrus, T. J. Earley, A. Patapoutian, Neuron 64, 498 (Nov. 25, 2009)), although the majority of Piezo1 expression (similar to TRPA1) is present inside the cell. This demonstrates that Piezo1 protein can be localized at or near the plasma membrane (FIG. 9B). The antibody was not sensitive enough to detect endogenous N2A expression of Piezo1 (data not shown).

### Example 8

### Piezo2 is Required for DRG Rapidly-Adapting MA Currents

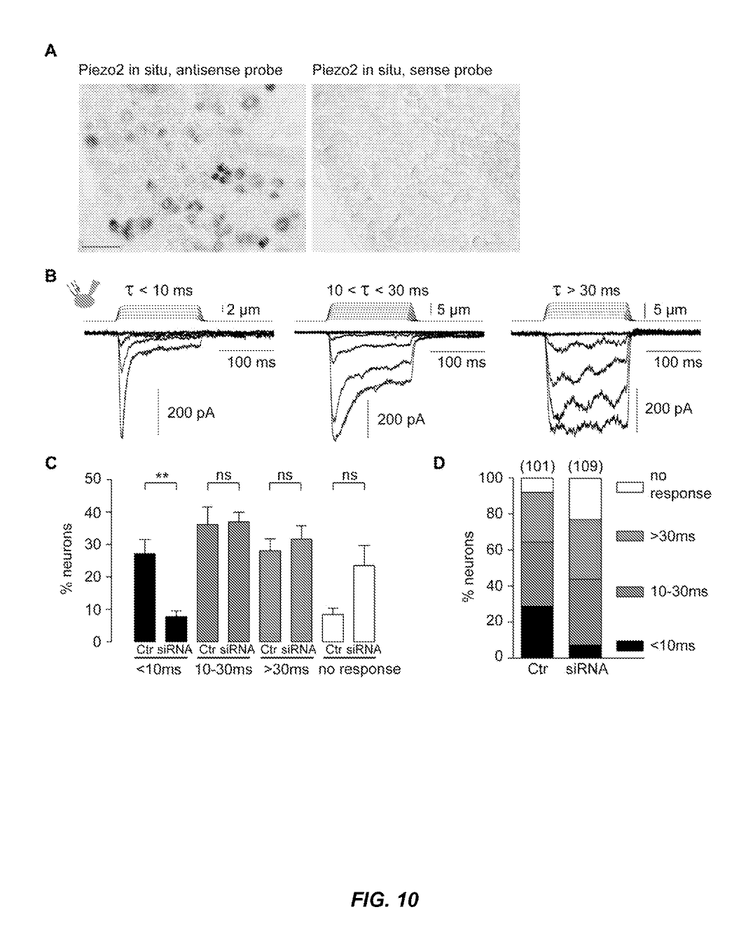

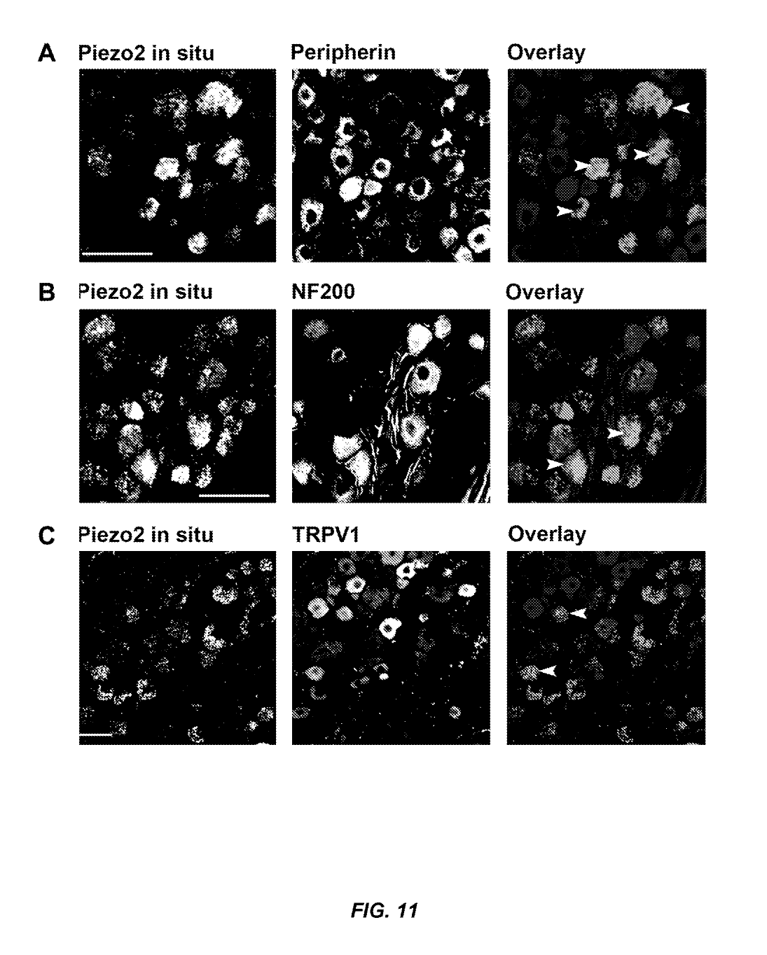

Piezo2 but not Piezo1 is expressed at relatively high levels in DRGs as assessed by qPCR (FIG. 4C). To characterize Piezo2 expression within the heterogeneous population of neurons and glial cells of the DRGs, in situ hybridization was performed on adult mouse DRG sections (FIG. 10A). Piezo2 mRNA expression was observed in 20% of DRG neurons (from 2391 total neurons—see methods section below). Piezo2 is expressed in a subset of DRG neurons also expressing peripherin (60%) and Neurofilament 200 (28%), markers present in mechanosensory neurons (FIG. 11) (M. E. Goldstein et al., J Neurosci Res 30, 92 (September, 1991); S. N. Lawson, Exp Physiol 87, 239 (March, 2002); S, N. Lawson et al., J Comp Neurol 228, 263 (Sept. 10, 1984); H. Sann et al., Cell Tissue Res 282, 155 (October, 1995)). Some overlap with nociceptive marker TRPV1 (24%) further suggests a potential role of Piezo2 in noxious mechanosensation.

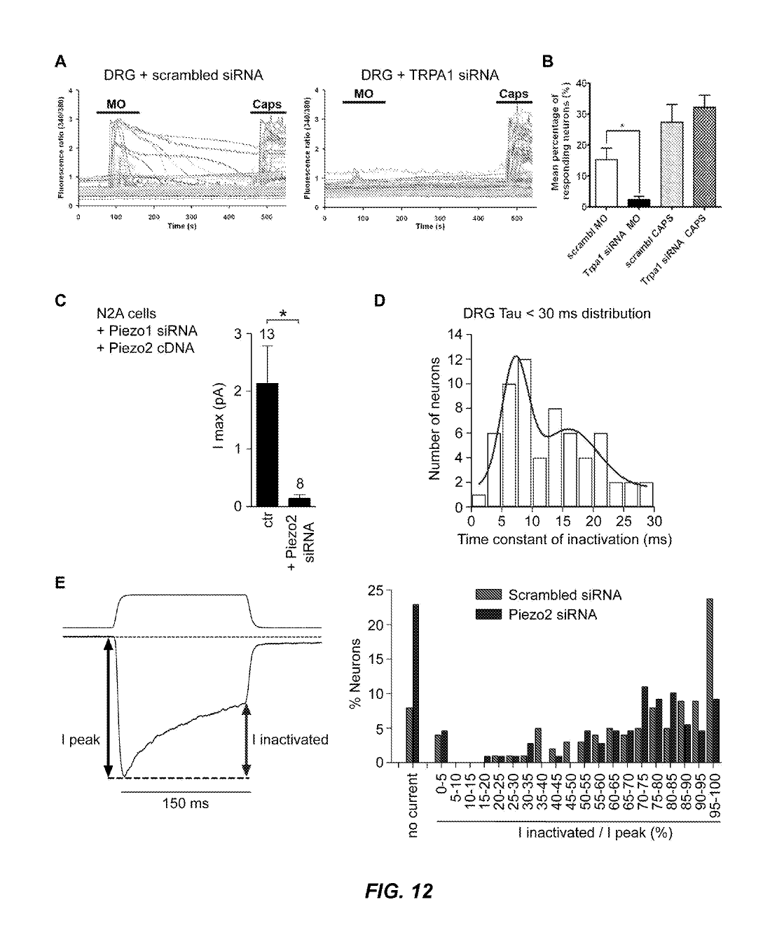

Next, the role of Piezo2 in MA currents of DRG neurons was directly examined using siRNA transfection. The RNAi approach was first validated on TRPA1, an ion channel expressed in DRG neurons and activated by mustard oil (MO) (M. Bandell et al., Neuron 41, 849 (Mar. 25, 2004); S. E. Jordt et al., Nature 427, 260 (Jan. 15, 2004)) (FIG. 12A-B). The ability of siRNAs to block functional expression of Piezo2 was demonstrated in N2A cells co-transfected with both Piezo2 cDNA and Piezo2 siRNA (FIG. 12C, 15-fold decrease). Then whole-cell MA currents were recorded from DRG neurons co-transfected with GFP and either scrambled or Piezo2 siRNA (n=101 for scrambled, and n=109 for Piezo2 siRNA). The recorded MA currents were grouped according to their inactivation kinetics, as previously described (FIG. 10B) (B. Coste et al., J Gen Physiol 129, 57 (January, 2007); L. J. Drew et al., J Neurosci 22, RC228 (Jun. 15, 2002); L. J. Drew et al., J Physiol 556, 691 (May 1, 2004); J. Hu, G. R. Lewin, J Physiol 577, 815 (Dec. 15, 2006); C. Wetzel et al., Nature 445, 206 (Jan. 11, 2007)). Four different classes of neurons were defined based on τ_{inac} distribution in scrambled siRNA transfected cells (FIG. 6D): τ_{inac}<10 ms, 10<τ_{inac}<30, τ_{inac}>30, and non-responsive neurons. Piezo2-expressing neurons with T_{inac}˜7 ms (FIG. 7) would be expected within the τ_{inac}<10 ms DRG population. Remarkably, the proportion of neurons expressing MA currents with τ_{inac}<10 ms was specifically and significantly reduced in Piezo2 siRNA compared to scrambled siRNA transfected neurons (FIG. 10C). 28.7% of scrambled siRNA transfected neurons had τ_{inac}<10 ms versus 7.3% in Piezo2 siRNA transfected neurons (FIG. 10D). Neurons with MA currents with slower kinetics (τ_{inac} between 10 and 30 ms and τ_{inac}>30 ms) were present at normal populations in Piezo2 siRNA samples. A trend towards increased numbers of mechanically insensitive neurons was observed, as predicted if loss of Piezo2 converts rapidly adapting neurons into non-responders. These RNAi data also were analyzed according to the degree of current inactivation during the 150 ms test pulse and came to similar conclusions (FIG. 12E). It is unknown whether the remaining MA currents with τ_{inac}<10 ms are due to incomplete Piezo2 knockdown or the presence of another channel complex. Regardless, Piezo2 is specifically required for the majority of the rapidly adapting MA ion channel activity in cultured DRGs.

### Example 9

### Therapeutic Benefit of Targeting of Piezo1 and Piezo2

Many studies have implicated calcium-permeable mechanically-activated (MA) cationic currents in vertebrate mechanotransduction (O. P. Hamill, B. Martinac, Physiol Rev 81, 685 (April, 2001)). Cell-based mechanical sensitivity was assayed here using two different, well-established methods: a piezoelectrically-driven pressure applied from a glass probe in whole-cell conformation, and stretch of the plasma membrane through a patch pipette in cell-attached mode (J. Hao et al., in Mechanosensitivity of the Nervous System, I. K. e. A. Kamkin, Ed. (Springer Netherlands, 2008), vol. 2, pp. 51-67). Using these assays, Piezo1 was found to be required for MA currents in Neuro2A cells, and Piezo2, for a subset of MA currents in DRG neurons. Moreover, overexpressing Piezo1 or Piezo2 in three different cell types gives rise to a remarkable 17-300 fold increase in MA currents. Therefore, Piezos are both necessary and sufficient for the expression of a MA current in various cell types. Notably, Piezo1 or Piezo2 overexpression confers unique adaptation properties of MA currents, arguing that they are components of distinct MA ion channels.

Piezo1 and Piezo2 sequences appear unique, not resembling known ion channels or other protein classes. The very large number of predicted transmembrane domains (30 and 34 transmembranes for mouse Piezo1 and Piezo2, respectively) is reminiscent of voltage-activated sodium channels with 24 transmembrane domains, composed of a 4-fold repeat of 6-transmembrane units (M. R. Hanlon, B. A. Wallace, Biochemistry 41, 2886 (Mar. 5, 2002)). However, pore-containing or repetitive domains have not initially been observed in Piezo proteins. It may be that Piezo proteins are non-conducting subunits of ion channels required for proper expression or for modulating channel properties, similar to beta subunits of voltage-gated channels (M. R. Hanlon, B. A. Wallace, Biochemistry 41, 2886 (Mar. 5, 2002)) or SUR subunits of ATP-sensitive K+ channels (S. J. Tucker, F. M. Ashcroft, Curr Opin Neurobiol 8, 316 (June, 1998)). This is unlikely, since it would imply that all the cell types used here express a silent conducting subunit of an MA channel that requires Piezos to function. Alternatively, Piezo proteins may define a novel class of ion channels, akin to Orail, a recently identified ion-conducting channel without significant homology to previously known channels (M. Prakriya et al., Nature 443, 230 (Sep. 14, 2006)). Piezo1/Fam38A has also been found in the endoplasmic reticulum (K. Satoh et al., Brain Res 1108, 19 (Sep. 7, 2006); B. J. McHugh et al., J Cell Sci 123, 51 (Jan. 1, 2010)), so Piezos may act at both the plasma membrane and in intracellular compartments. Indeed, the data here have shown that overexpressed Piezo1 can be observed at or near the plasma membrane.

Piezo1 is expressed in a variety of tissues involved in mechanotransduction, including in the kidney. Interestingly, stretch-activated channels with similar properties have been described in kidney-derived cells (R. Sharif-Naeini et al., J Mol Cell Cardiol 48, 83 (January, 2010); P. Gottlieb et al., Pflugers Arch, (Oct. 23, 2007)). Piezo1 expressed sequenced tags (ESTs) are also found in the inner ear. The conductance of MA channels of hair cells varies according to location in the cochlea, ranging from 80-163 pS (A. J. Ricci et al., Neuron 40, 983 (Dec. 4, 2003)). Although this range does not resemble that conducted via Piezo1, the variability in conductance suggests it may be modulated by yet unknown factors, and therefore a candidate should not be excluded on this basis.

Piezo2 is expressed in sensory neurons and is required for mechanically-activated currents. Mechanical hyperalgesia is a condition prevalent in many pain conditions including inflammatory and neuropathic pain. Therefore, Piezo2 can be a target for a variety of pain, itch, and inflammation indications. The targeting of Piezo1 and Piezo2 could also have therapeutic benefit in a variety of indications including hearing, adjustment of vascular tone and blood flow, urine flow sensing in kidney, lung growth and injury, as well as bone and muscle homeostasis, all of which are all regulated by mechanotransduction.

### SEQUENCE LISTING

A sequence listing is submitted along with this application as an ASCII text file entitled 87396-817888_ST25.txt. This file was created on Aug. 23, 2011 and is 139 kilobytes.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1-45. (canceled)

46. A method of ameliorating pain in a subject, the method comprising administering to the subject a) an antibody that antagonizes the activity of a mechanically activated cation channel polypeptide of SEQ ID NO:2, SEQ ID NO:4, SEQ ID NO:18, or SEQ ID NO:20, or b) an antisense oligonucleotide or small interfering RNA (siRNA) complementary to at least 15 contiguous nucleotides of SEQ ID NOs 1, 3, 17, or 19 wherein the antisense oligonucleotide or siRNA inhibits production of the mechanically activated cation channel polypeptide.

47. The method of claim 1, wherein the polypeptide comprises SEQ ID NO:4 or SEQ ID NO:20.

48. The method of claim 1 wherein the polypeptide is expressed in bladder, colon, kidney, lung, or skin.

49. The method of claim 1, wherein the polypeptide is expressed in a dorsal root ganglion neuron.

50. The method of claim 1, wherein the subject is a mammal.

51. The method of claim 1, wherein the subject is a human.

52. The method of claim 1, wherein the pain is selected from the group consisting of acute mechanical pain, chronic mechanical pain, mechanical hyperalgesia, mechanical allodynia, arthritis, inflammation, dental pain, cancer pain, and labor pain.

53. The method of claim 1, wherein the antibody is a monoclonal antibody, a humanized antibody or a chimeric antibody.

54. The method of claim 1, wherein the antisense oligonucleotide or siRNA comprises any one of SEQ ID NOs 5-16.

55. An isolated antisense oligonucleotide or small interfering RNA (siRNA) complementary to at least 15 contiguous nucleotides of SEQ ID NOs:1, 3, 17, or 19 and encodes a mechanically-activated cation channel polypeptide, wherein the antisense oligonucleotide or siRNA inhibits production of the mechanically-activated cation channel polypeptide.

56. The isolated antisense oligonucleotide or siRNA of claim 10, wherein the antisense oligonucleotide or siRNA comprises any one of SEQ ID NOs:5-16.

57. An expression cassette comprising a promoter operably linked to a polynucleotide comprising the antisense oligonucleotide or siRNA of claim 10.

58. A vector comprising the expression cassette of claim 12.

59. A cell comprising the expression cassette or expression vector of claim 13, wherein the expression cassette or expression vector is heterologous to the cell.

60. An antibody that antagonizes the activity of a mechanically-activated cation channel selected from SEQ ID NO:2, SEQ ID NO:4, SEQ ID NO:18, or SEQ ID NO:20.

61. The antibody of claim 15, wherein the antibody is a monoclonal antibody, a humanized antibody or a chimeric antibody.

62. A method of screening for an agent that modulates the activity of a mechanically-activated cation channel, the method comprising:
contacting a mechanically-activated cation channel polypeptide having at least 70% amino acid sequence identity to at least one of SEQ ID NOs:2, 4, 18, or 20 with an agent; and
selecting the agent that modulates the activity of the mechanically-activated cation channel polypeptide.

63. The method of claim 17, wherein the activity of the mechanically-activated cation channel polypeptide is determined by measuring an electrophysiological change mediated by the polypeptide comprising any of a change in membrane potential, a change in current, an influx of a cation, or a mechanically activated electrophysiological change; and wherein measuring optionally comprises any of:
i) measuring a membrane potential with a membrane potential dye,
ii) measuring an electrophysiological change with a patch-clamp assay, and
iii) measuring a mechanically activated electrophysiological change.

64. The method of claim 17, wherein the polypeptide is expressed in a cell and the contacting comprises contacting the cell with the agent; wherein the cell optionally:
i) comprises a heterologous expression cassette comprising a promoter operably linked to a polynucleotide encoding the mechanically-activated cation channel polypeptide;
ii) is a eukaryotic cell;
iii) is a neuron; or
iv) is in an animal.

65. The method of claim 17, wherein the polypeptide comprises SEQ ID NO:2, SEQ ID NO:4, SEQ ID NO:18, or SEQ ID NO:20.
